(19) United States
(12) Patent Application Publication
(10) Pub. No.: US 2026/0120793 A1
(43) Pub. Date: Apr. 30, 2026

(54) METHOD FOR IDENTIFYING SPECIFIC DRUG CANDIDATES FOR DISEASE TREATMENT AND USE OF A METHOTREXATE OR METRONIDAZOLE FOR THE TREATMENT OF A PATIENT WITH A NEUTROPHILIC INFLAMMATION-MEDIATED DISORDER

(71) Applicant: National Yang Ming Chiao Tung University, Taipei (TW)

(72) Inventors: Tai-Ming KO, Taipei City (TW); Jan Vincent B. BELTRAN, Taipei City (TW); Fang-Ping LIN, Taipei City (TW); Chaw-Liang CHANG, Taipei City (TW); Che-Ming LIN, Taipei City (TW)

(21) Appl. No.: 18/932,826

(22) Filed: Oct. 31, 2024

(51) Int. Cl. G16B 15/30; G16B 40/30; G16C 20/40; G16H 20/10

(52) U.S. Cl. CPC G16B 15/30; G16B 40/30; G16C 20/40; G16H 20/10

(57) ABSTRACT

The present invention provides a drug identifying method designed to evaluate specific candidate drugs for targeted diseases, identifying those most effective for treating the condition. And a new indication of the selected drugs methotrexate or metronidazole for the treatment of diseases mediated by neutrophilic inflammation targeting the aberrant CD177⁺ neutrophil subpopulation.

### FIELD OF THE INVENTION

The present invention relates to a drug identifying method designed to evaluate specific candidate drugs for targeted diseases, identifying those most effective for treating the condition. The selected drugs, methotrexate or metronidazole, are particularly applied in treating patients with diseases mediated by neutrophil-driven inflammation.

### BACKGROUND OF THE INVENTION

Kawasaki disease (KD) and multisystem inflammatory syndrome in children (MIS-C) are pediatric hyperinflammatory syndromes characterized by uncontrolled immune responses. KD was first described in 1967, whereas MIS-C was first identified in 2020 during the COVID-19 pandemic. A plethora of infectious agents have been proposed to trigger KD, including viral, fungal, and bacterial agents, whereas MIS-C emerges≈4 to 6 weeks after a SARS-COV-2 infection. KD and MIS-C share similar clinical manifestations, including persistent fever, rash, conjunctivitis, lymphadenopathy, and cardiac disorders. Among the shared cardiovascular complications, coronary artery aneurysm (CAA) and myocarditis can lead to myocardial infarction and sudden death. Compared with KD, MIS-C exhibits more severe morbidity: In addition to multiorgan dysfunction, up to 80% of patients exhibit cardiological issues, including aneurysm, myocarditis, left ventricular dysfunction, arrhythmia, and cardiogenic shock, all of which contribute to increased mortality rates.

Although some studies have proposed that KD and MIS-C are distinct diseases, others have proposed that they share similar host responses related to a hyperinflammatory state. KD and MIS-C have recently been indicated as syndromes on the same host immune response continuum. Hence, on the basis of all this information, KD and MIS-C may be driven by a conserved immunopathological mechanism controlled by the same molecular drivers. Diseases with the same molecular drivers can be attenuated using the same pharmacological strategies. Therefore, identification of the shared regulators of KD and MIS-C could accelerate the development of therapeutic interventions. KD treatments have been clinically effective in ameliorating MIS-C. MIS-C has been treated with intravenous immunoglobulin (IVIG), steroids, and drugs to attenuate inflammation and prevent molecular damages. However, although most patients respond to IVIG and glucocorticoids as first-line therapeutic strategies, a fraction of patients with KD and MIS-C have proven resistant and refractory. This resistance confers a high risk of CAA formation in ˜25% and 14% to 48% of patients with KD and MIS-C, respectively. As heart-related pathologies are a principal cause of death in KD and MIS-C, patients in critical care settings require more intensive therapeutic strategies to support heart function and resolve multiple organ comorbidities.

Food and Drug Administration (FDA)—approved drugs for KD and MIS-C unfortunately are limited. Therefore, there is an urgent need to develop drugs that can effectively ameliorate the pathogenic immune dysregulation in KD and MIS-C.

Despite advances in KD and MIS-C research, the complex dysregulated immune state associated with their development remains poorly understood. Myeloid activation has been actively investigated; however, clinical studies have also noted the distinct increase in blood neutrophil counts in patients with KD and MIS-C that is speculated to contribute to pathogenesis, but the evidence is lacking. Although neutrophils play a crucial role in shaping innate and adaptive responses, a dysregulated neutrophil activation state may impair host responses, especially during infection. Neutrophils are critical innate effector cells that are activated in the earliest phases of inflammation and infection; they constantly circulate throughout the body and rapidly kill pathogens through phagocytosis, degranulation, neutrophil extracellular trap (NET) formation, and reactive oxygen species (ROS) production. Owing to its circulatory distribution, migratory advantage, and effector repertoire, the abnormal activation of neutrophil processes can lead to excessive and systemic damage depending on its cellular state. As such, high neutrophil counts and dysfunction can contribute to disease pathogenesis and are recognized as drivers of morbidity and mortality in certain diseases.

The molecular mechanisms underlying KD and MIS-C neutrophilia and its role in the immunopathologies shared by both diseases have not been fully elucidated at the single-cell level. Bulk transcriptomics may undermine gaining a mechanistic understanding in contexts with heterogeneous sample origins, such as with blood, which contains various immune cells. With single-cell RNA sequencing (scRNA-seq), specific cell types can be characterized and their roles in disease development can be elucidated. However, the following issues limit the characterization of neutrophils using standard scRNA-seq protocols and pipelines:
(1) neutrophil depletion steps are performed during peripheral blood mono-nuclear cell (PBMC) preparation because they are the most abundant circulating leukocyte in blood, which may mask the biological effects of other important cell types;
(2) neutrophils are short-lived and should thus be analyzed within ˜2 hours of collection;
(3) neutrophils are not optimized for 10× Genomics Cell Ranger algorithms, hence requiring special workflows;
(4) neutrophils are a transcriptionally quiescent cell type (they have low RNA and high RNase content), hence strict preprocessing quality control procedures may remove them from the data; and
(5) there are clustering and annotation challenges to differentiating neutrophils from monocyte lineages. These factors result in low neutrophil recovery for downstream applications; hence, neutrophil-optimized protocols are needed. Another issue in neutrophil studies is the lack of sample size (both patients and healthy controls, especially in pediatric medicine), which makes the interpretation of small-sample analyses challenging. To circumvent these limitations, we conducted an unbiased single-cell meta-analysis and a systems-level integrative analysis of neutrophil activation across multiple cohorts and contexts.

Methotrexate is an immunosuppressant approved for the therapeutic treatment of rheumatoid arthritis, psoriasis, and other forms of cancer. It has been researched in KD patients; however, the exact mechanism is unknown. Our invention uses methotrexate to specifically target CD177⁺ neutrophils in KD. In addition, while methotrexate is approved for arthritis, it is not approved for both KD and MIS-C treatment.

Additionally, these are not yet approved for other neutrophil-mediated immunological diseases including COVID-19, neutrophilic asthma, chronic obstructive pulmonary disease (COPD), systemic lupus erythematosus, biliary atresia, sepsis, inflammatory bowel disease, neutrophilic dermatoses, chronic neutrophilic leukemia (CNL), juvenile myelomonocytic leukemia (JMML), cytokine storm, acute respiratory distress syndrome (ARDS), bronchiectasis and cystic fibrosis (CF).

The present invention aimed to investigate the role of neutrophils and the mechanisms underlying the shared immuno-pathogenesis of KD and MIS-C. We performed a single-cell meta-analysis of neutrophil activation by integrating several cohort studies, identifying a neutrophil subpopulation that is only associated with KD and MIS-C compared with other pediatric diseases with similar clinical symptoms. Our integrative computational analysis identified shared regulatory genes that may be used as novel drug targets or diagnostic markers and determined FDA-approved drugs that may be tested for fast clinical translation. Our findings highlight the undocumented role of CD177⁺ neutrophils as a potential pathogenic driver of KD and MIS-C development, clinically relevant to their systemic and multiorgan comorbidities.

In present invention methotrexate as a method to treat neutrophilic inflammation by targeting the S100A12 in CD177⁺ neutrophils. Similarly, we propose metronidazole to treat neutrophilic inflammation by targeting the TSPO in CD177⁺ neutrophils.

### SUMMARY OF THE INVENTION

Given the problem mentioned earlier, the present invention provides a method of identifying specific drug candidates for disease treatment and a Drug Identification Module.

Embodiments of the method and the Drug Identification Module can include any one or more of the following features.

### Data Pre-Processing

Newly generated single-cell RNA-seq data or existing data obtained from public genomic repositories, including disease and control groups, are loaded into R/RStudio and processed using Seurat package. Individual datasets are pre-processed according to a series of quality control steps. It involves filtering cells based on the nCount_RNA, nFeatures_RNA, mitochondrial content, doublets/multiplets, and other low quality-correlated genes that can lead to confounding effects. Standard scRNA-seq data pre-processing workflows are also performed, including the functions NormalizeData, Find VariableFeatures, ScaleData, RunPCA, and RunUMAP.

### Integration and Clustering

All the pre-processed datasets are then merged, and the standard data processing workflow was repeated as merging data erases all normalized and scaled data. The merged data is integrated using Harmony package while performing necessary batch corrections. To prepare the integrated data for cell type identification, clustering analysis is performed via FindNeighbors( ) and FindClusters( ) algorithms.

### Cell Annotation

Using the integrated data with defined clusters, specific cell identities are classified using automated cell annotation or reference mapping. In this step, the clusters are assigned a specific cell identity.

### Pathogenic Cell Type Identification

Since the identities of the clusters in the data are now known, they are going to be assessed which one is implicated in the disease. For this, we assessed: (a) differential abundance, (b) activation state, and (c) expression of gene signatures associated with the morbidities/pathologies of the disease. This step ranks the cell types and nominates one that is most implicated in disease development. However, not all in this specific cell type collectively drive the pathogenicity. Often, they differentiate or produce abnormal subsets of the cell type which can drive pathogenesis. To define the specific cell subtype, the major cell type is isolated from the integrated data, and further re-clustering is conducted to define subclusters. Similar evaluation is conducted on the subclusters in terms of differential abundance, activation state, and expression of risk gene signatures, in comparison with healthy group, which ultimately identifies the likely pathogenic, abnormal cell subtype.

### Identifying the Molecular Mechanisms Driven by the Pathogenic Cell Subtype

Differential expression analysis is performed comparing the transcriptome of the pathogenic cell subtype vs healthy controls, generating disease-associated gene expression signatures. Via pathway enrichment analysis, we determined the pathways and processes activated by the pathogenic cell subtype to promote disease development. These abnormally activated, disease-associated pathways will be used later for correlating with the lead target. Via disease-gene association network analysis, we further confirm the potential of the pathogenic cell subtype to trigger specific pathologies/morbidities involved in the disease development.

### Identifying the Critical Modulators of Disease Mechanisms

Via high-dimensional weighted gene coexpression network analysis, we identified specific genetic modules composed of highly interacting genes across cells. These modules include key hub genes that are most important and biologically relevant to the functions of the pathogenic cell subtype. Since this step can identify the central regulators in a transcriptional regulatory program, identifying the lead target from this module is very important. A target prioritization strategy is performed to narrow down the druggable lead targets that may regulate the pathogenic regulatory mechanisms. The target expression is correlated with the expression of the relevant diseases mechanisms/pathways as well as clinical stages to support its value as a therapeutic target.

### Drug Identification

Using the identified most important lead target(s), we conducted cheminformatics approach to nominate drugs based on the DGIdb (Drug-Gene Interaction Database) which calculates query score and interaction score. The first part is to do the drug-gene interaction analysis which mines the druggable genome across drug databases included in DGIdb, only including FDA-approved drugs for the purpose of drug repositioning. We finalize the candidate drugs according to this set of criteria: (i) drugs targeting the disease-associated regulatory program, (ii) drugs targeting the key hub genes in disease-associated cell subtype, (iii) drugs targeting the important genes implicated by disease-gene association network analysis, (iv) drugs targeting the genes correlated with the disease-associated pathways, (v) drugs that are non-agonist/activating/potentiating and with DGIdb interaction score >0.2. The second part involves the molecular docking (structure-based blind docking) of the identified candidate drugs to the lead drug target(s). First the 3D protein structures of drug targets are obtained from RCSB Protein Data Bank, from Homo sapiens if human protein data exists, otherwise an analogue version from Mus musculus is used. On the other hand, SDF chemical structures of drugs are obtained from EMBL-EBI Chemical Entities of Biological Interest or the Chemical Compounds Deep Data Source, wherever the chemical structure is available. Discovery Studio software is used to preprocess the protein structures in preparation for docking. We utilized the CB-DOCK2 docking server to perform structure-based cavity-guided blind docking. The binding affinities are automatically generated, and the drugs are ranked accordingly.

The present invention further provides a method for therapeutic treatment of a patient with a neutrophilic inflammation-mediated disorder, the method comprises administering to the subject a drug, wherein the drug can target S10012 or TSPO proteins, wherein the drug is selected from a Methotrexate or a Metronidazole.

In one embodiment, the drug can target to an immune cell, which is CD177⁺ neutrophils.

In one embodiment, the methotrexate targets the S100A12 protein.

In one embodiment, the metronidazole targets the TSPO protein.

In one embodiment, a combination of the methotrexate and the metronidazole target the S100A12 and TSPO, respectively.

In one embodiment, the invention exhibits suppressive action against CD177⁺ neutrophils when the methotrexate or the metronidazole (alone or in combination) inhibit S100A12 and/or TSPO.

In one embodiment, the methotrexate or the metronidazole can attenuate the CD177⁺ neutrophil mechanisms: neutrophil hyperactivation, neutrophil degranulation, neutrophil transendothelial transmigration, neutrophil extracellular trap (NET) formation, and reactive oxygen species/reactive nitrogen species (ROS/RNS) production.

In one embodiment, persistent fever is treated.

In one embodiment, coronary artery aneurysm is treated.

In one embodiment, myocarditis is treated.

In one embodiment, myocardial infarction is treated.

In one embodiment, neutrophilic inflammation is treated.

In one embodiment, respiratory/pulmonary inflammation is treated.

In one embodiment, asthma is treated.

In one embodiment, sepsis is treated.

In one embodiment, cytokine storm is treated.

In one embodiment, vascular inflammation is treated.

In one embodiment, any of the neutrophilic inflammation-mediated disorder is treated.

In one embodiment, the patient has increased levels of neutrophils higher than normal neutrophil counts.

In one embodiment, the treatment strategy suppresses CD177⁺ neutrophil-targeted pathways, including: CD177⁺ neutrophil expansion, neutrophil hyperactivation, neutrophil degranulation, neutrophil transendothelial transmigration, neutrophil extracellular trap (NET) formation, and reactive oxygen species/reactive nitrogen species (ROS/RNS) production, to substantially control the hyperactivated neutrophils in order to ameliorate, prevent, or inhibit systemic inflammation, organ damage, and cardiovascular disorders.

In one embodiment, the targets (S100A12 and TSPO) are critical hub genes common to mechanisms as above.

In one embodiment, the patient has or is predisposed to symptoms of a neutrophilic inflammation-mediated disorder.

In one embodiment, the drug is as a disease modulating strategy rather than a symptomatic treatment.

In one embodiment, the drug further can combine with one or more immunomodulatory or neutrophil-targeted drugs.

In one embodiment, the neutrophilic inflammation-mediated disorder is a neutrophil-related pathology or disease, wherein the neutrophil-related pathology or disease comprises Vasculitis, Kawasaki disease, Multisystem inflammatory syndrome in children, COVID-19, Neutrophilic Asthma, Chronic obstructive pulmonary disease (COPD), Systemic lupus erythematosus, Biliary atresia, Sepsis, Inflammatory bowel disease, Neutrophilic dermatoses, Chronic neutrophilic leukemia (CNL), Juvenile myelomonocytic leukemia (JMML), Cytokine storm, Acute respiratory distress syndrome (ARDS), Periodontitis, Bronchiectasis, or Cystic fibrosis (CF).

### BRIEF DESCRIPTION OF THE DRAWINGS

FIG. 1A-1G show the overview of the single-cell meta-analysis of neutrophil activation across pediatric health conditions.

FIG. 2A-2D show the distribution of nFeature_RNA and nCount_RNA in Neutrophils of HC, KD, and MISC stages and the sub-clustering of neutrophil subpopulations across all conditions. A. Distribution in HC, KD_A, and KD_IVIG. B. Distribution in HC, MIS-C_M, MIS-C_S and MIS-C_R. C. Distribution in neutrophil subclusters (0, 1, 2, 3). D. Neutrophil subpopulations across conditions/diseases.

FIG. 3 show the abundance and significance of the results of differential abundance testing of cell types and neutrophil subpopulations in KD and MIS-C, compared to HC. A-B. LogFC and FDR significance visualization across cell types in KD (A) and MIS-C(B). C-D. LogFC and FDR significance visualization across neutrophil subpopulations in KD (C) and MIS-C (D).

FIG. 4 show the neutrophils' increased expression of signatures associated with risks of cardiovascular events. A. Signature associated with acute CVD (cardiovascular disease). B. Signature associated with MACLE (major adverse cardiovascular or limb events). C. Signature associated with MI (myocardial infarction).

FIG. 5A-5K show the expansion of a highly activated CD177⁺ neutrophil subpopulation in both KD and MIS-C.

FIG. 6A-6B show the identification of CD177⁺ neutrophils. A. Identification of the top surface marker in neutrophil subcluster 0. B. Correlation of transcriptomes of CD177⁺ neutrophils in KD and MIS-C.

FIG. 7A-7P show the shared neutrophil expression program (SNEP) in CD177⁺ neutrophils conferring elevated neutrophil effector functions.

FIG. 8A-8F show the pathway enrichment in KD neutrophil clusters 2 and 3, compared with cluster 1. A-C.KD cluster 2 pathways in REACTOME (A), KEGG (B), and GO: BP (C). D-F. KD cluster 3 pathways in REACTOME (D), KEGG (E), and GO: BP (F).

FIG. 9A-9F show the pathway enrichment in MIS-C neutrophil clusters 2 and 3, compared with cluster 1. A-C. MIS-C cluster 2 pathways in REACTOME (A), KEGG (B), and GO: BP (C). D-F. MIS-C cluster 3 pathways in REACTOME (D), KEGG (E), and GO: BP (F).

FIG. 10A-10I show the determination of the master regulator of the SNEP in KD and MIS-C by transcription factor-regulon analysis.

FIG. 11 shows the transcription factor analysis. A. TFs inferred via Cytoscape iRegulon analysis and the upregulated TFs in KD and MIS-C. Red color indicates common to all groups, bold black for TFs shared between two groups, and plain black text for TFs unique to the group. B. Co-expression analysis of upregulated SNEP TFs (HMGB2, NFE2, HCLS1, and BCL6) with CD177.

FIG. 12A-12F show the results of hdWGCNA. A. Selection of the soft power threshold. B. Visualization of genes in modules ranked by kME. C-F. Pearson correlation of NM-4 module with neutrophil pathways (A) neutrophil degranulation, (B) ROS/RNS production, (C) transendothelial migration, and (D) NET formation.

FIG. 13A-13L show the identification of hub genes correlated with neutrophil activation using single-cell level high-dimensional weighted gene coexpression network analysis (hdWGCNA).

FIG. 14A-14J show the association of SNEP of KD and MIS-C with cardiovascular disorders, KD IVIG treatment, and MIS-C severity and recovery.

FIGS. 15A˜15F show FeaturePlot visualization of the expression of the 26 members of the Shared_CVD (aside from TSPO and S100A12). Each gene is visualized across three groups, HC, KD, and MIS-C.

FIG. 16A-16F show the repurposing FDA-approved drugs to target the shared immunopathologies of KD and MIS-C

FIG. 17A-17J show the validation from independent cohorts of scRNA-seq (KD) and RNA-seq (MIS-C). A. UMAP visualization of the cell types of the integrated scRNA-seq data of KD cohort. B. Comparative feature plot visualization showing the expansion of CD177⁺ neutrophils in the KD group. C-D. Volcano plot result of differential expression analysis in KD (C) and MIS-C(D). CD177, S100A12, and TSPO are all significantly upregulated. E-G. Upregulation of CD177, S100A12, and TSPO in KD compared to HC in single-cell RNA-seq. H-J. Upregulation of CD177, S100A12, and TSPO in MIS-C compared to HC in bulk RNA-seq data.

FIG. 18A-18D show the validation from independent scRNA-seq cohorts of adult systemic vasculitis diseases. A. UMAP visualization of the integrated scRNA-seq data of the Behcet's Disease (BD) cohort. B. Comparing the CD177 gene expression in HC vs BD using feature plot visualization. Gray color indicates no expression. C. UMAP visualization of the integrated scRNA-seq data of the Giant Cell Arteritis (GCA) cohort. D. Comparing the CD177 gene expression in HC vs GCA using feature plot visualization. Gray color indicates no expression.

FIG. 19 shows the prepared structures of the drug targets as well as the drug 3D chemical structures. A. Cartoon backbone representation of the prepared structure of S100A12 (PDB ID: 2wce). B. Cartoon backbone representation of the prepared structure of TSPO (PDB ID: 2mgy). C. 3D structure of methotrexate. D. 3D structure of zaleplon. Protein targets were visualized via CD-BOCK2 while drugs were visualized using Discovery Studio.

FIG. 20 shows the top 3 results of molecular docking of methotrexate into the binding site of S100A12. A. Top 1 docking pose (C5) and the corresponding zoomed visualization of methotrexate at the binding site. B. Top 2 docking pose (C4) and the corresponding zoomed visualization of methotrexate at the binding site. C. Top 3 docking pose (C3) and the corresponding zoomed visualization of methotrexate at the binding site.

FIG. 21 shows the top 3 results of molecular docking of zaleplon into the binding site of TSPO. A. Top 1 docking pose (C1) and the corresponding zoomed visualization of zaleplon at the binding site. B. Top 2 docking pose (C2) and the corresponding zoomed visualization of zaleplon at the binding site. C. Top 3 docking pose (C4) and the corresponding zoomed visualization of zaleplon at the binding site.

### DETAILED DESCRIPTION OF THE INVENTION

### Definition

CAA refers to coronary artery aneurysm; CD177 refers to Cluster of Differentiation 177; DNV refers to Dengue virus infection; FDA refers to Food and Drug Administration; JIA refers to juvenile idiopathic arthritis; KD refers to Kawasaki Disease; MIS-C refers to multisystem inflammatory syndrome in children; NET refers to neutrophil extracellular trap; PBMC refers to peripheral blood mononuclear cells; PCD refers to pediatric celiac disease; RNS refers to reactive nitrogen species; ROS refers to reactive oxygen species; S100A12 refers to S100 Calcium-binding Protein A12; scRNA-seq refers to single-cell RNA sequencing; SNEP refers to shared neutrophil expression program; SPI1 refers to Spi-1 proto-oncogene; TF refers to transcription factor; TSPO refers to translocator protein.

In the present invention, drug identification is based on “structure-based molecular docking”. “The single-cell meta-analysis was used to identify important genes or pathogenic drivers that can serve as drug targets, and the protein structures of these genes were utilized for drug repurposing”. For the drug repurposing process, we initially used publicly available protein structures of the target genes from the existing PDB (Protein Data Bank) database. For example, as stated on page 23 of the specification, the protein target IDs extracted from the public database include: TSPO protein target PDB ID: 2mgy and S100A12 protein target PDB ID: 2wce.

Additional specific embodiments of the present invention include, but are not limited to the following:

### Methods

All studies were approved by the National Yang Ming Chiao Tung University institutional review board under NCTUREC-109-036F. All the scRNA-seq PBMC data used in this study are publicly available: GSE168732, GSE167029, GSE183716, GSE152450, GSE166489, GSE184330, GSE205095, GSE154386, GSE200743, GSE217370, GSE198616, and GSE19889132 (Tables 1-3). In brief, we integrated and harmonized 9 cohorts with healthy control (HC), KD, MIS-C, dengue virus infection (DNV), juvenile idiopathic arthritis (JIA), and pediatric celiac disease (PCD) pediatric data. We interrogated the neutrophils through a series of computational analyses, including re-clustering, differential expression analysis, pathway analysis, high dimensional weighted gene coexpression network analysis, cardiovascular disease association network analysis, correlation analysis, and transcription factor-regulon analysis. We assessed its heterogeneity to identify a potentially pathogenic neutrophil subpopulation and investigated the transcriptional programs and regulators shared in KD and MIS-C that are relevant to systemic damage and cardiac pathologies. Key transcriptional features and signatures in KD and MIS-C were compared with DNV, JIA, and PCD. For validation, we confirmed the upregulation of CD177⁺ neutrophils and targets by using independent cohorts of PBMC scRNA-seq for KD and neutrophil bulk RNA-seq for MIS-C. Furthermore, we analyzed independent cohorts of adult-onset vasculitis (Behcet disease and giant cell arteritis) to gain insights on the pediatric disease specificity of CD177⁺ neutrophils. We conducted drug-gene interaction network and molecular docking analyses to identify FDA-approved drugs that may be tested for the attenuation of both KD and MIS-C by targeting its shared mechanisms and immunopathologies. Complete and detailed methods are provided in Supplemental Methods.

TABLE 1
Summary of scRNA-seq public data from 9 cohorts (discovery)

| | | | | | No. of |
|---|---|---|---|---|---|
| NCBI GEO ID | Journal Publication | PMID | Disease | Stage | patients |
| GSE152450 | Journal of | 33758528 | HC | HC | 2 |
| | Inflammation | | KD | KD_A | 2 |
| | Research (2021) | | | | |
| GSE166489 | Immunity (2021) | 33891889 | HC | HC | 6 |
| | | | MIS-C | MISC_R | 2 |
| | | | | MIS-C_M | 2 |
| | | | | MIS-C_S | 4 |
| GSE167029 | Med (2021) | 34414385 | HC | HC | 6 |
| | | | KD | KD_A | 3 |
| | | | MIS-C | MIS-C_M | 2 |
| | | | | MIS-C_S | 6 |
| GSE168732 | Nature Communications | 34521850 | HC | HC | 3 |
| | (2021) | | KD | KD_A | 6 |
| | | | | KD_IVIG | 6 |
| GSE183716 | iScience (2021) | 34632327 | KD | KD_A | 1 |
| | | | MIS-C | MISC_R | 1 |
| | | | | MIS-C_M | 2 |
| GSE184330 | Journal of | 34914824 | HC | HC | 4 |
| | Experimental | | MIS-C | MIS-C_MS | 4 |
| | Medicine | | | MIS-C_M | 2 |
| | (2022) | | | | |
| GSE205095 | N/A | N/A | HC | HC | 2 |
| | | | JIA | JIA_NULL | 6 |
| | | | | JIA_TNF | 6 |
| GSE154386 | PLoS Pathogens (2021) | 33513191 | DNV | 2_DBD | 4 |
| | | | | 3_DBD | |
| | | | | 4_DBD | |
| | | | | 5_DBD | |
| Ramirez-Sanchez, | Frontiers in | 35371081 | HC | HC | 10 |
| et al (Author- | Immunology (2022) | | PCD | PCD | 11 |
| provided) | | | | | |

| | |
|---|---|
| TOTAL | 103 |

TABLE 2
Summary of public data from two independent
cohorts of KD and MIS-C (validation)

| NCBI | Journal | Single-cell | | Dis- | Sam- |
|---|---|---|---|---|---|
| GEO ID | Publication | Library | PMID | ease | ples |
| GSE200743 | Frontiers in | 10x | 36159873 | KD | 3 |
| | Immunology | Genomics | | | |
| | (2022) | Chromium | | | |
| GSE217370 | Cell Reports | 10x | 36476388 | HC | 12 |
| | Medicine | Genomics | | MIS-C | 17 |
| | (2022) | Chromium | | | |

| | |
|---|---|
| TOTAL | 32 |

TABLE 3
Summary of public data from two independent
cohorts of BD and GCA (validation)

| NCBI | Journal | Single-cell | | Dis- | Sam- |
|---|---|---|---|---|---|
| GEO ID | Publication | Library | PMID | ease | ples |
| GSE198616 | Proceedings of | 10x | 35727985 | HC | 4 |
| | the National | Genomics | | BD | 4 |
| | Academy of | Chromium | | | |
| | Sciences (2022) | | | | |
| GSE198891 | Rheumatology | 10x | 35460236 | HC | 3 |
| | (Oxford) (2022) | Genomics | | GCA | 3 |
| | | Chromium | | | |

| | |
|---|---|
| TOTAL | 14 |

### Statistical Methods

All statistical methods were performed in R v.4.2.2 and RStudio v.2022.02.2+485 “Prairie Trillium.” Differential expression analysis was performed using the Seurat v.4.3.0 package FindAllMarkers( ) unction and Wilcoxon rank sum test with the following thresholds: log 2fold change >0.25, min.pct>0.25, min.diff.pct>0.5, and adjusted P value <0.05. Similar thresholds were set for identifying differentially expressed transcription factors (TFs), except log 2fold change >0.20. Pathway enrichment significance was assessed through the EnrichR web server with adjusted P value <0.05. For statistical comparisons of mean differences between 2 groups, we performed the Wilcoxon rank sum test, and Kruskal-Wallis test for comparing >2 groups. Pearson correlation analyses were performed using ggcorrplot v.0.1.4 package of ggplot2 v.3.4.0. Significance was determined if the P value was <0.01, unless otherwise stated.

### Example 1

### Computational Method on Lead Target and Drug Identification from Single-Cell Transcriptomics Meta-Analysis of the Drug Identification Module

### Data Acquisition

Obtained the data (Table 1-2) from the Gene Expression Omnibus (GEO) repository of the National Center for Biotechnology Information (NCBI) or obtained online when included in the article's attachments according to the following criteria: (1) pediatric age, 0-18 y/o; (2) available scRNA-seq data; (3) PBMC (peripheral blood mononuclear cells) sample. We collected the PBMC single-cell data from pediatric healthy control (HC), Kawasaki disease (KD), and Multi-system inflammatory syndrome in children (MIS-C). KD samples included acute and post-IVIG treatment subgroups, while MIS-C samples included moderate, severe, and recovery stages. We also extracted other pediatric scRNA-seq data of diseases including Dengue Virus Infection (DENV), Juvenile Idiopathic Arthritis (JIA), and Pediatric Coeliac Disease (PCD) for comparison and independent cohorts of KD and MIS-C for validation. To assess the pediatric specificity of CD177⁺ neutrophils in KD and MIS-C, we further included PBMC scRNA-seq data from adult-onset systemic vasculitis syndromes, Behcet's Disease (BD) and Giant Cell Arteritis (GCA) (Table 3). Due to computational challenges, data that contain less <200 cells were aggregated together with other samples that belong to the same condition.

### Pre-Processing and Quality Control

We loaded the datasets in R v.4.2.2/RStudio v.2022.02.2+485 “Prairie Trillium” and re-processed them using the Seurat v.4.3.0, retaining a minimum of 3 cells with >200 features. We filtered cells with nFeatures_RNA<5,000, nCount_RNA<20,000, <5% hemoglobin genes, and <15% mitochondrial genes. Low quality-correlated gene MALATI and sex-associated genes XIST and RSP4Y1 were filtered out to prevent confounding effects. To circumvent the ribosomal genes' technical bias, they were also removed. Next, we utilized DoubletFinder60 (https://github.com/chris-mcginnis-ucsf/DoubletFinder) v.2.0.3 to remove doublet cells. All datasets were independently processed. In brief, the method is carried out with the following methods: NormalizeData, FindVariableFeatures (selection.method= “vst”, nfeatures=3000), ScaleData, RunPCA, and RunUMAP (dims=1:10). Multiplet rates were adjusted depending on the total number of cells where nExp parameter was calculated from. Finally, doublets were computed using doubletFinder( ) function, setting the parameters to pN=0.25, pK=0.09, the computed nExp, and Pcs=1:10, followed by removing the cells identified as doublets from the dataset. Then, we performed cell cycle scoring using the Seurat built-in gene sets of S and G2M phases to infer cell cycle phase and regress its effects in downstream analyses.

### Data Integration and Clustering

To enable integration, we first aggregated all the datasets using the merge( ) function. Since merging individual normalized data erases all normalized and scaled data, we repeated the standard Seurat data processing workflow. We normalized the data, calculated the top 3,000 highly variable genes using the “vst” selection method, scaled the data, and performed dimensionality reduction by PCA and UMAP embedding. We then integrated all data using the Harmony v.0.1.1 package61, while setting the disease groups in RunHarmony( ) group.by.vars parameter to enable batch correction. We obtained the harmony embeddings, performed UMAP and clustering using the harmony embeddings (reduction= “harmony”) instead of “pca.” We included the first 25 dimensions for RunUMAP and FindNeighbors, then used FindClusters (Louvain algorithm) to define the clusters at resolution=0.5.

### Cell Annotation

Utilizing SignacX v.2.2.5 (https://mathewchamberlain.github.io/SignacX/), a neural network-based machine learning classifier trained from the Human Primary Cell Atlas62, we identified the cell types and cell states. Following its vignette, we generated the SignacX labels for our Seurat object by running SignacFast( ) and GenerateLabels( ) Afterwards, we appended the cell type and cell state classifications to the metadata.

### Differential Abundance Testing

To identify whether the cell types have differential abundance during diseased state, we performed a differential abundance testing following the tutorial from http://bioconductor.org/books/3.13/OSCA.multisample/differential-abundance.html #performing-the-da-analysis. We compared the statistical differences of cell type abundances in KD and MIS-C in comparison with the HC across individual levels to identify cluster abundance. We determined the significant cluster abundance at FDR<0.05.

### Neutrophil Subsets Re-clustering

We subsetted the neutrophils, performed re-clustering, and harmonization. To do this, we identified the top 3,000 highly variable genes using Find VariableFeatures (selection.method= “vst”) and performed scaling using ScaleData( ) To identify the major neutrophil subsets and perform batch correction, we conducted the following steps: RunPCA (npcs=5), RunHarmony (group.by.vars= “group”, theta=2), RunUMAP (reduction= “harmony”, dims=1:5), and FindClusters (resolution=0.1).

### Identification and Definition of Shared Neutrophil Expression Programs (SNEP)

We confirmed that the transcriptomes of CD177⁺ neutrophils in KD and MIS-C are similar by calculating the average expressions of CD177⁺ neutrophils' transcriptomes in both KD and MIS-C and plotted the correlation using FeatureScatter( ) To facilitate the identification of key targets common to KD and MIS-C, we defined the “shared expression neutrophil expression programs (SNEP)” as the upregulated genes found in the hyperactivated neutrophil subpopulation in both KD and MIS-C whose expression is associated with acute stages, IVIG treatment, severity, and recovery. To identify the SNEP in CD177⁺ neutrophils of KD and MIS-C, we first conducted differential expression analysis in HC (cluster 1, CD177⁻) vs KD (cluster 0, CD177⁺) or MIS-C (cluster 0, CD177⁺), then filtered the overlap of the resulting upregulated genes of KD and MIS-C CD177⁺ neutrophils. We used the FindAllMarkers( ) function, selecting only the upregulated genes, setting the minimum expression to at least 25% of the cells, min.diff.pct=0.25, and log 2FC=0.5. We used Enhanced Volcano v.1.14.0 to generate the volcano plots. SNEP was the intersection of genes upregulated in KD (KD_UP) and MIS-C(MIS-C_UP) identified using VennDiagram v.1.7.3.

### Pathway Enrichment Analysis

We utilized EnrichR63 web server (accessed 11/2022) to do the pathway analysis, including the Gene Ontology, REACTOME Gene Sets, ad KEGG Pathways. We analyzed the shared pathways activated in CD177⁺ neutrophils of KD and MIS-C. Meanwhile, we utilized Metascape64 web server (accessed 11/2022) to construct the protein-protein interaction network of the SNEP. This is carried out using the STRING, BioGrid, OmniPath, and InWeb_IM databases, and employs the MCODE (Molecular Complex Detection) algorithm for physical interaction networks containing 3 to 500 proteins which deduces densely connected network components.

### Disease-Gene Association Analysis

To test whether the SNEP is associated with cardiovascular pathologies, we utilized the DisGeNET65 v.7.0 from Cytoscape v.3.9.1. We narrowed down the disease associations by setting the class to “Cardiovascular Diseases” to limit the disorders relevant cardiac phenotypes in KD and MIS-C. Specifically, we filtered the genetic associations to (i) coronary and (ii) myocardial diseases. We retained the other parameters as default. We obtained the genes that are associated with both coronary and myocardial phenotypes and scored it as a gene signature (labeled as Shared_CVD) for comparison against HC, KD, and MIS-C and for subsequent pathway enrichment analysis.

### Transcription Factor (TF) Regulon Analysis

Via Cytoscape66 v.3.9.1, we conducted the iRegulon v.1.3 Analysis67 to analyze TFtarget interactions. Using the SNEP as input gene list, we predicted the transcriptional regulators with the following default settings: Ranking (type of search space: genebased, motif collection: 10K (9713 PWMs), track collection 1120 ChIP-seq tracks (ENCODE raw signals), putative regulatory region: 20 kb centered around TSS, motif rankings database: 20 kb centered around TSS (7 species), track rankings database: 20 kb centered around TSS (ChIP-seq-derived)), Recovery (enrichment score threshold: 3.0, ROC threshold for AUC calculation: 0.03, rank threshold: 5000), and TF prediction (minimum identity between orthologous genes: 0.0, maximum false discovery rate (FDR) on motif similarity: 0.001). We assessed the most important regulators by filtering TFs with NES>3.0 and ranking them according to regulon size, expression levels across groups, and correlation with neutrophil activation and effector pathways. Meanwhile, we also identified the upregulated TFs in KD and MIS-C from differential expression analysis using the following thresholds: log 2FC>0.20, min.pct>0.25, min.diff.pct>0.25, and adjusted p-value <0.01.

High-Dimensional Weighted Gene Co-Expression Network Analysis (hdWGCNA)

To perform co-expression network analysis on single-cell data, we used the hdWGCNA v.0.2.1 package68. We setup the neutrophils Seurat object for WGCNA, selecting genes that are expressed in at least 5% of the dataset using SetupForWGCNA( ) with gene_select= “fraction”. Since correlation network approaches like WGCNA are sensitive to data sparsity, we opted to use the built-in metacells functionality instead of the original single cells. Metacells are defined as aggregates of small groups of cells with similar transcriptomic profile that originate from same biological sample. We constructed the metacell expression matrices of the neutrophils via the function MetacellsByGroups( ) then normalized it using NormalizeMetacells( ) Prior to constructing the network, we transposed the metacell expression matrix since WGCNA requires the columns to be genes as opposed to standard Seurat objects where rows are genes and columns are cells. We tested different soft powers and selected the optimal soft power threshold, which, according to hdWCGNA documentation, is the lowest soft power threshold that has a Scale Free Topology Model Fit greater than or equal to 0.8. Our analysis identified the soft power threshold as 8 and used this as parameter to ConstructNetwork( ) We visualized the co-expression modules via a dendrogram, where colored leaves are true modules and grey modules are genes that were not classified into any co-expressed modules, hence were not relevant in downstream analyses.

### Modules Scoring and Correlation Analysis

Gene signatures and modules were scored using the AddModuleScore( ) function of Seurat to calculate disease signatures, cell-level module activity, or pathway activation scores. Neutrophil pathways scored include neutrophil activation involved in immune response (GO: 0002283), Neutrophil degranulation (REACTOME R-HSA-6798695), RHO GTPase activates NADH oxidase (REACTOME R-HSA-5668599), Leukocyte Transendothelial Migration (KEGG hsa-04670, and Neutrophil Extracellular Trap Formation (KEGG hsa-04613). For correlation analysis, we applied Pearson correlations using ggcorrplot v.0.1.4 package of ggplot2 v.3.4.0.

### Drug-Gene Interaction Analysis

To identify druggable targets in CD177⁺ neutrophils, we utilized the Drug-Gene Interaction database (DGIdb v4.2.0) 69 which mines the druggable genome across drug databases including the DrugBank, PharmGKB, ChEMBL, Drug Target Commons, Therapeutic Target Database (TTD), and others. We filtered the drug-gene interactions to include only FDA-approved drugs which can be readily translated to clinic. The regulatory approval status of the drugs is from ChEMBL database. To search for an interaction, DGIdb calculates two scores: query score and interaction score. These two scores are automatically computed based on evidence score, relative gene specificity, and relative drug specificity. Lastly, we finalized the most promising drugs based on this criterion: (i) drugs targeting the SNEP, (ii) drugs targeting the hub genes in hyperactivated neutrophils, (iii) drugs targeting the Shared_CVD, (iv) drugs with targets that are co-expressed with CD177, (v) dugs with targets that are correlated with neutrophil activation and aberrant effector pathways, (vi) drugs that are nonagonist/activating/potentiating, with DGIdb interaction score >0.2.

### Molecular Docking

To verify whether the suggested drugs can bind to the drug targets, we conducted structure-based blind docking. We tested the top 1 suggested drugs per drug target: methotrexate for S100A12 and zaleplon for TSPO. We first obtained the 3D protein structures of the drug targets from the RCSB Protein Data Bank: 2mgy (TSPO) and 2wce (S100A12). Since there is no available TSPO structure for humans, we obtained the one from Mus musculus, whereas the data for S10012 is from Homo sapiens. We then obtained the SDF chemical structures of methotrexate (C₂₀H₂₂N₈O₅, ChEBI ID: 44185) from the EMBL-EBI Chemical Entities of Biological Interest (ChEBI) database and of zaleplon (C₁₇H₁₅N₅O) from the CHEMICAL COMPOUNDS DEEP DATA SOURCE (https://www.molinstincts.com/). Using Discovery Studio 2021 Client (v.21.1.0.2029B), we preprocessed the protein structures and removed the water molecules to prepare for docking. In the case of TSPO, since there are 20 conformations in the 2mgy structure, we used model 1 for our assessments. For S100A12, we merged the two chains A and B to generate the full structure ready for docking. Through CBDOCK270, a protein-ligand docking server that combines cavity detection, structure-based docking, and template-based docking and is based on AutoDock Vina, we performed cavity-guided blind docking.

### Independent Cohort Validation

The expansion of CD177⁺ neutrophils and upregulation of major targets (S100A12 and TSPO) was validated using two independent cohorts: GSE200743 for KD scRNA-seq and GSE217370 for MIS-C bulk RNA-seq. In addition, we also analyzed independent cohorts of adult-onset vasculitis such as BD (GSE198616) and GCA (GSE198891) to gain insights into the potential pediatric disease specificity of KD and MIS-C.

For validation in KD using scRNA-seq data, we considered some factors from the modified analysis pipeline of Wigerblad et al71 for processing scRNA-seq data for neutrophil analysis. Their thresholds may improve detection of neutrophils in healthy, steady state contexts; however, some of the upper thresholds may not be suitable for diseases characterized by hyperactivated neutrophil states. To confirm the consistency of our major findings while adapting their important recommendations, we applied a modified strategy for the validation analysis. In addition to the general pre-processing and quality control strategies we employed during the discovery stage, we retained cells expressing at least 100 cells for the validation study. After the neutrophils were extracted from the integrated data, we also removed cells expressing >1 copy of CD3G, HBA2, and RSP8, which are markers of T cells, erythrocytes, and rRNA predominance, respectively, to ensure high data quality. Since GSE200743's HC data were not pediatric, we used the pediatric controls from GSE166489 for comparison.

For validation in MIS-C, we obtained public bulk RNA-seq data which included high purity bulk neutrophil samples from pediatric HC and MIS-C patients. We re-analyzed the data following the RNA-seq analysis workflow of Love at al72. We performed the DESeq2 differential expression analysis to identify the upregulated genes and ensured consistent results from the original paper.

Similarly, using the same approach from the validation study of KD scRNA-seq mentioned above, we re-analyzed the BD and GCA scRNA-seq data independently to compare the specificity of CD177⁺ neutrophils in pediatric vs adult vasculitis.

### Example 2

### Single-Cell Meta-Analysis of Neutrophil Activation in KD and MIS-C

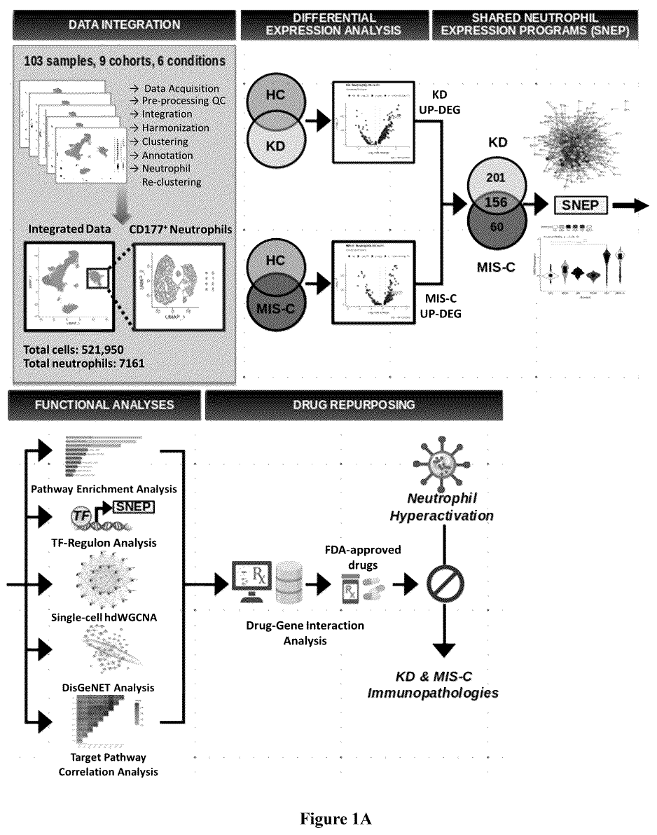

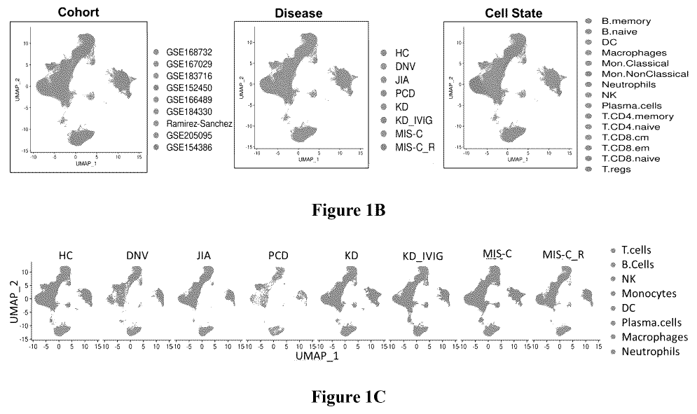

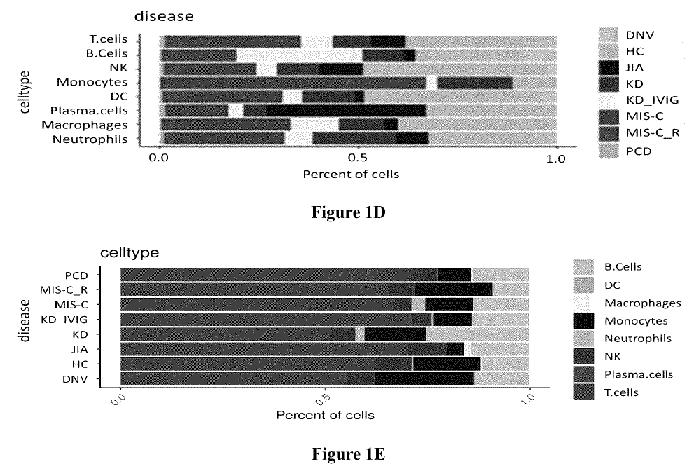

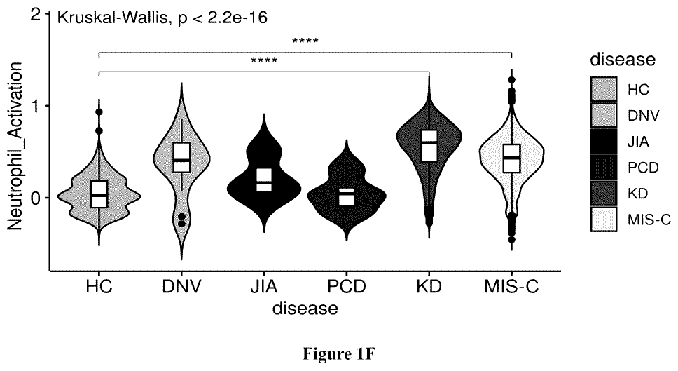

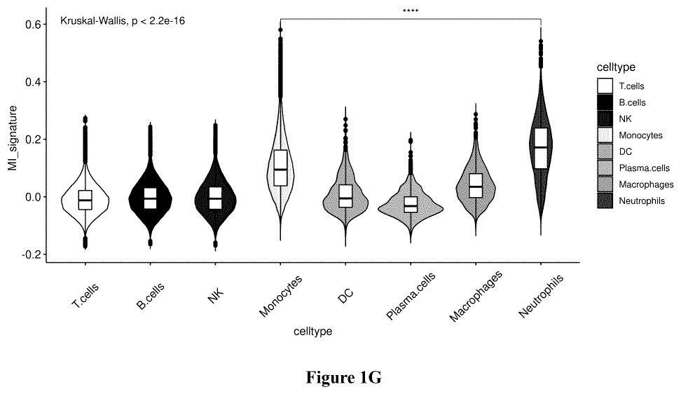

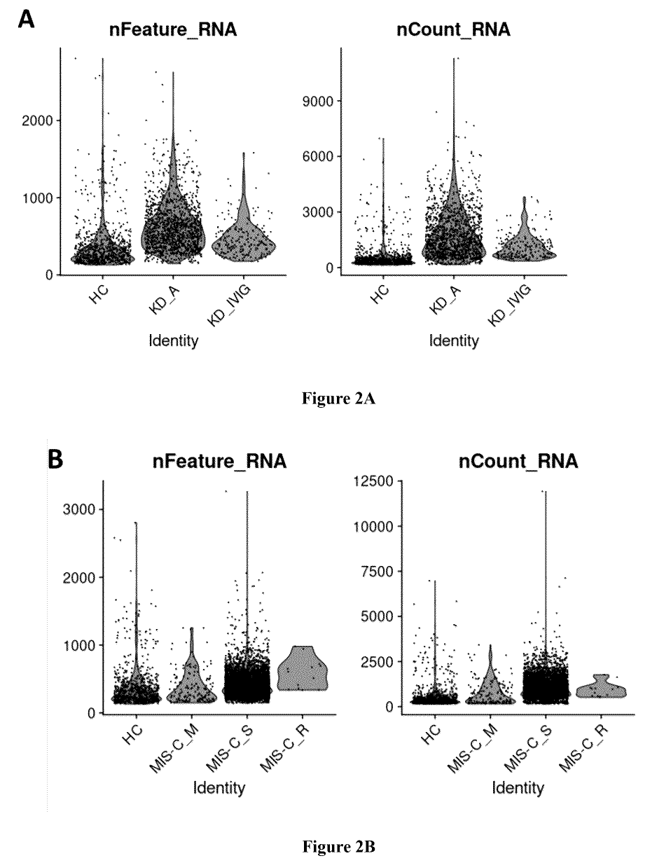

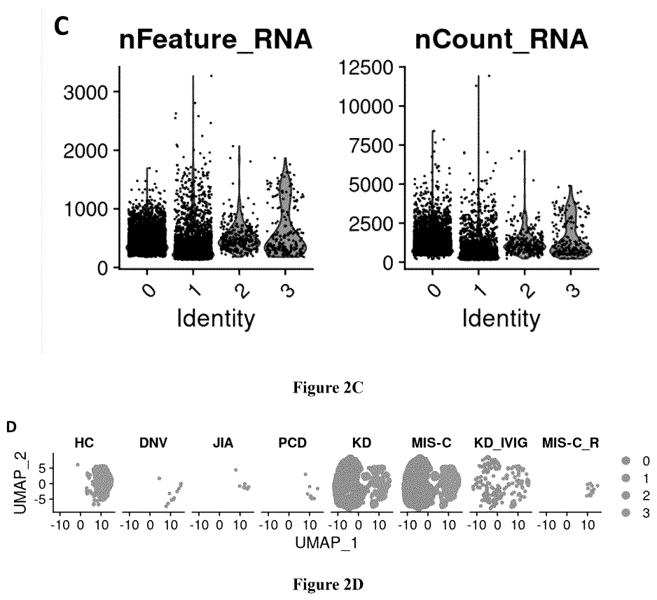

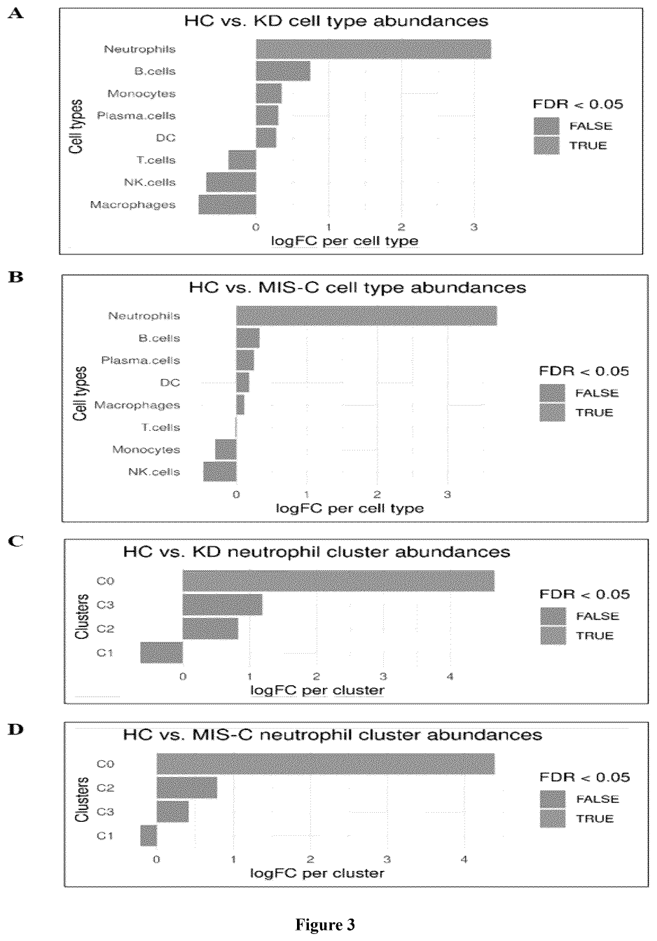

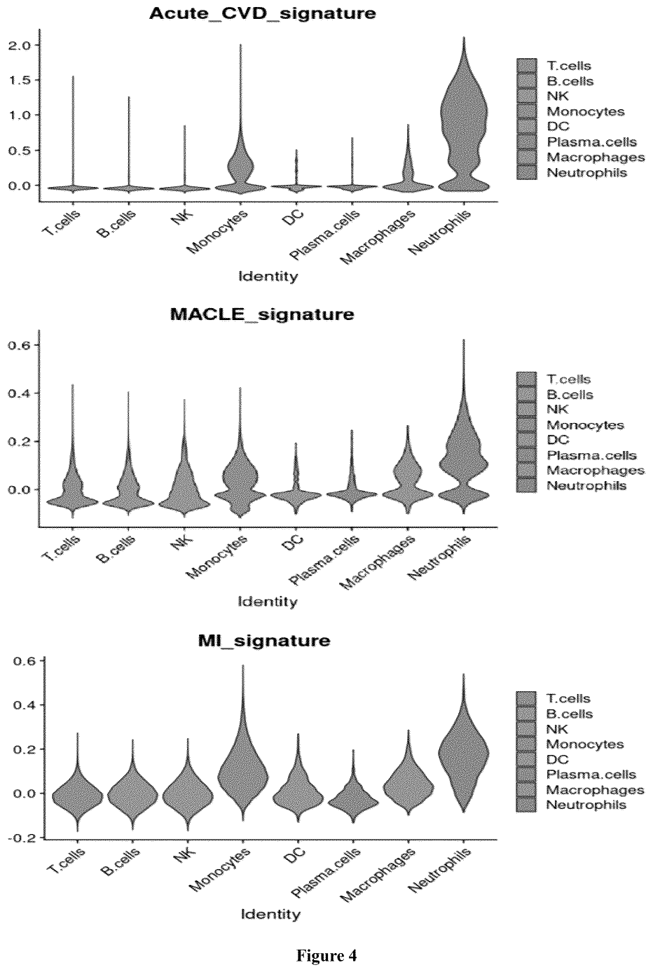

Meta-analyzed 103 pediatric single-cell transcriptomes across 9 studies, which included 33 HC, 18 KD (acute and IVIG-treated), 15 MIS-C(moderate, severe, and recovery), 4 DNV, 12 JIA, and 11 PCD samples (FIGS. 1A and 1C). After data processing, quality control, doublet filtering, aggregation, and harmonization, 521950 high-quality cells were retained and uniform manifold approximation and projection clustering resolved 35 clusters. SignacX annotated 18 cell states (FIG. 1B), including lymphoid (naive CD4⁺ T cells, memory CD4⁺ T cells, naive CD8⁺ T cells, effector memory CD8⁺ T cells, central memory CD8⁺ T cells, regulatory T cells, natural killer cells, naive B cells, memory B cells, and plasma cells), myeloid (classical monocytes, nonclassical monocytes, macrophages, and dendritic cells), epithelial, stromal (endothelial and fibroblast), and granulocyte (neutrophil) populations, but we filtered out the epithelial and stromal cells due to their low numbers. The expression profiles of the top-ranked cluster markers show canonical cell marker gene signatures indicating correct cell phenotype classification. We analyzed cell proportions across diseases and found that KD and MIS-C had the highest prevalence of neutrophils (FIGS. 1D and 1E; FIG. 2D; FIG. 3A; 2B), wherein MIS-C showed the highest neutrophil count. Across the 5 pediatric diseases, patients with KD and MIS-C showed the highest levels of neutrophil activation compared with healthy children (P<2.2e-16; FIG. 1F). Neutrophils had the highest expression of blood signatures related to future risks of cardiovascular events33-35 (FIG. 4A˜4C), followed by mono-cytes. Because we are investigating immune cells that may contribute to the development of its cardiovascular pathologies, we focused on studying neutrophil biology in the development and prognosis of KD and MIS-C.

### Example 3

### Expansion of a Highly Activated CD177± Neutrophil Subpopulation in Both KD and MIS-C

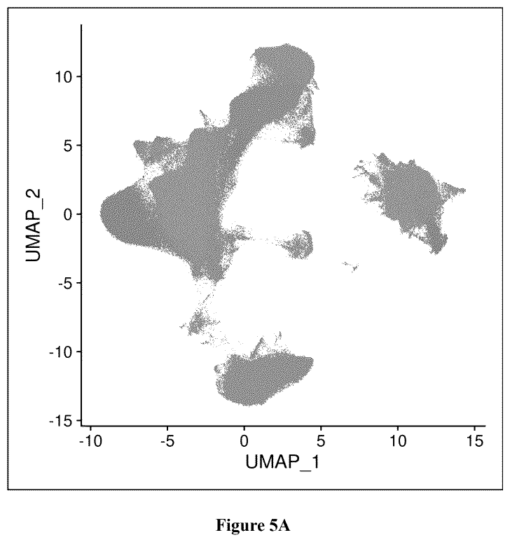

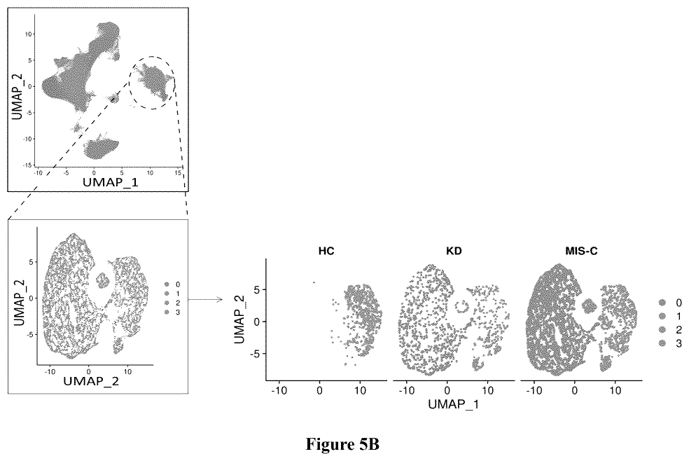

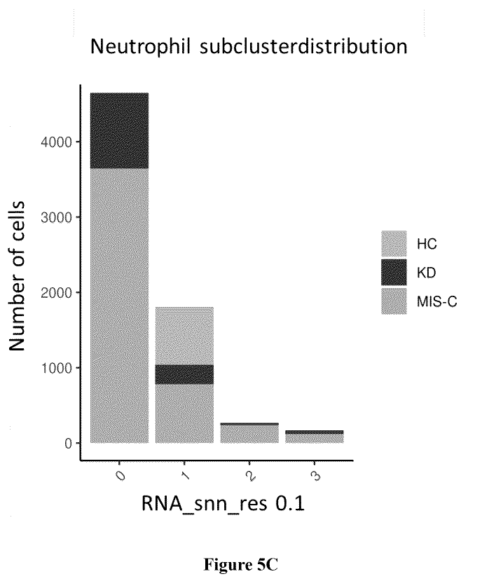

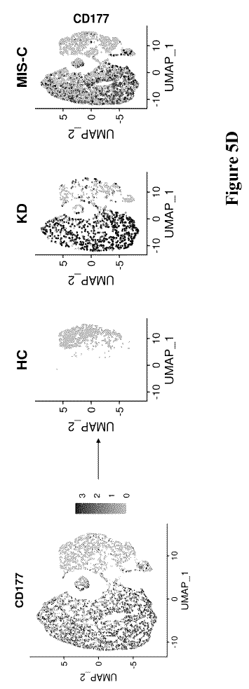

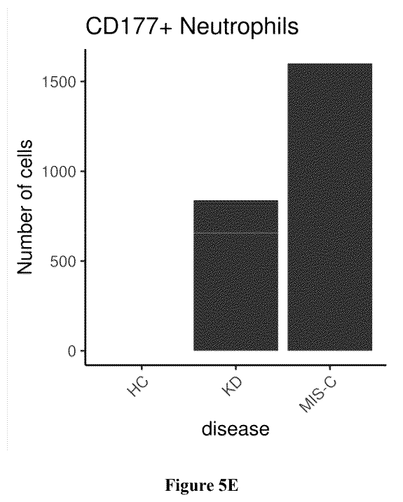

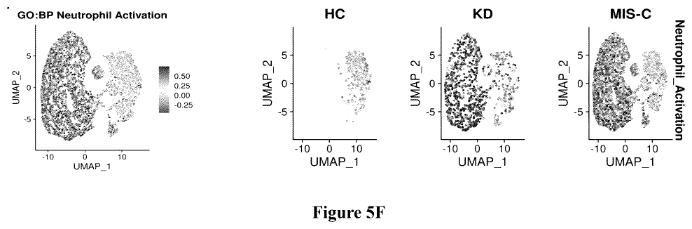

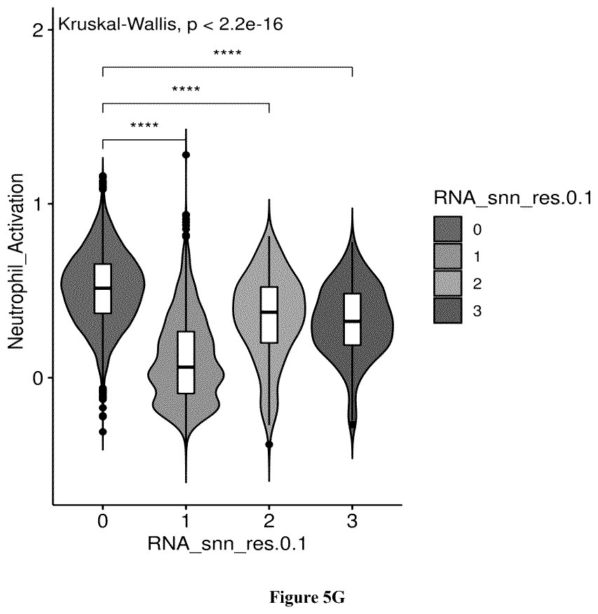

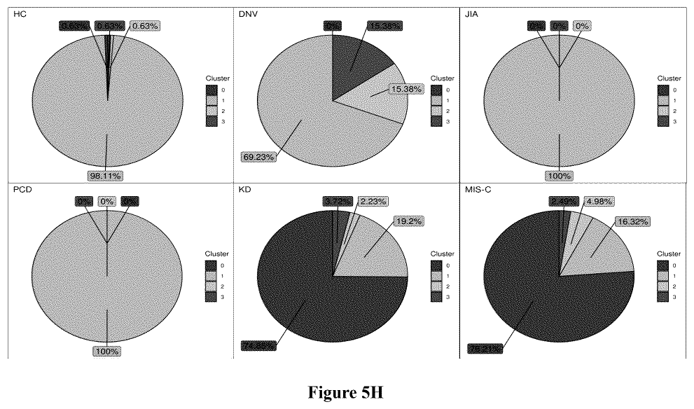

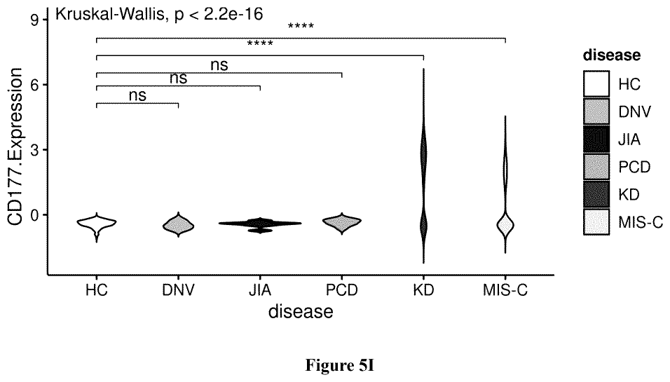

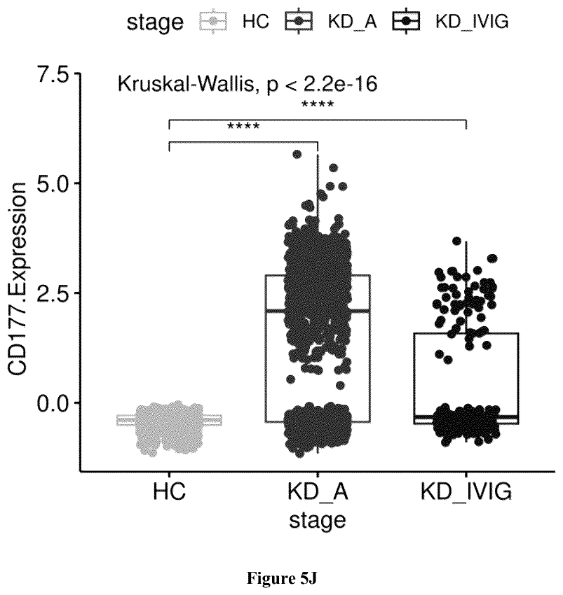

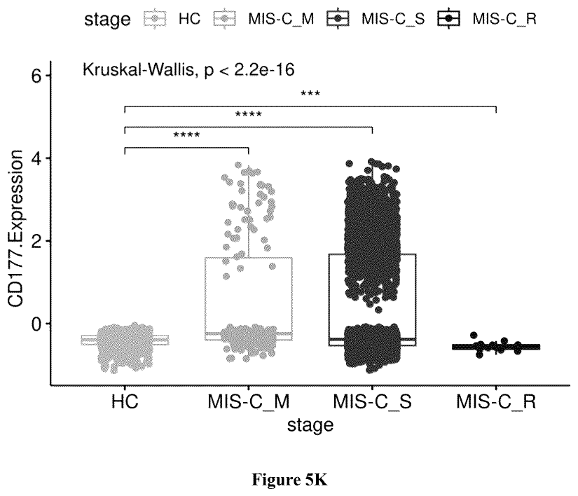

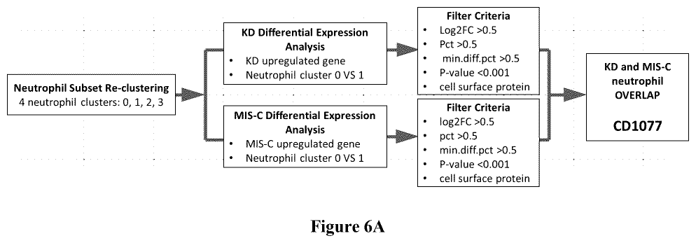

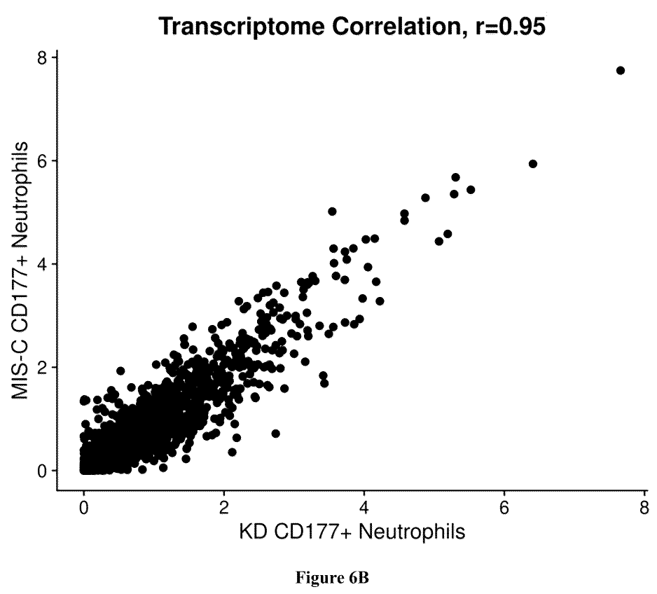

KD and MIS-C have marked elevation of neutrophil counts; however, its heterogeneity and clinical ramifications are largely undefined. We aimed to understand its pathological implications in KD and MIS-C. To identify disease-associated subpopulations, we recovered the 7161 single-cell transcriptomes of neutrophils across all conditions, performed re-clustering, and obtained 4 majors neutrophil subclusters: 0, 1, 2, and 3 (FIG. 5A˜5B; FIG. 2D), wherein cluster 0 expansion was only evident in KD and MIS-C(FIG. 2D; Tables 4˜7), whereas the HC group is overrepresented by cluster 1 (FIGS. 5B and 5C; Tables 4 and 5). Through differential abundance testing, we confirmed the significant expansion of neutrophils (false discovery rate <0.05; FIGS. 3A and 3B; Tables 8 and 9) and its subpopulation cluster 0 (false discovery rate <0.05; FIGS. 3C and 3D; Tables 10 and 11). We identified CD177 as the most dominant surface marker in cluster 0 neutrophils based on the following criteria: (1) log 2fold change >0.5; (2) min.pct>0.5 between HC and KD/MIS-C; (3) adjusted P value <0.001; and (4) a cell surface protein (FIG. 6A). CD177 neutrophils only exist in our KD and MIS-C data, but not in HC (FIGS. 5D and 5E). In addition, it is a highly activated neutrophil subpopulation (FIGS. 5F and 5G) that comprised ≈67% of the total neutrophil composition in KD and MIS-C, but not in other pediatric diseases (FIG. 5H). Although KD and MIS-C share some of the symptoms observed in other pediatric diseases with viral, immune, or inflammatory pathogenesis like DNV, JIA, or PCD, the expanded neutrophil population is only restricted to KD and MIS-C. Across the diseases, CD177 upregulation is highly associated with KD and MIS-C prognosis (P<2.2e-16), but not with DNV, JIA, and PCD (FIG. 5I˜5K). It is notable that patients with KD had lower percentages of CD177⁺ neutrophils after IVIG treatment (FIG. 5J). On the other hand, CD177⁺ neutrophils are depleted during MIS-C recovery (FIG. 5K). These data suggest an active role of neutrophils in these 2 diseases that warrants a comprehensive investigation.

TABLE 4
Summary of neutrophil cell counts per subpopulation
across HC, KD, and MIS-C patients

| Disease | Total | Neutrophil subpopulation | | | |
|---|---|---|---|---|---|
| group | Neutrophils | C0 | C1 | C2 | C3 |
| HC | 790 | 5 | 778 | 5 | 5 |
| | | (0.6%) | (98.1%) | (0.6%) | (0.6%) |
| KD | 1,344 | 1006 | 258 | 30 | 50 |
| | | (74.9%) | (19.2%) | (2.2%) | (3.7%) |
| MIS-C | 4,779 | 3642 | 780 | 238 | 119 |
| | | (76.2%) | (16.3%) | (5.0%) | (2.5%) |

TABLE 5
Summary of neutrophil cell counts per subpopulation across HC

| Study | | Total | Neutrophil Subpopulations | | | |
|---|---|---|---|---|---|---|
| Cohort | Dataset | Neutrophils | C0 | C1 | C2 | C3 |
| GSE166489 | HC4 | 3 | 0 | 3 | 0 | 0 |
| GSE166489 | HC5 | 4 | 0 | 4 | 0 | 0 |
| GSE166489 | HC6 | 6 | 0 | 6 | 0 | 0 |
| GSE166489 | HC7 | 7 | 0 | 7 | 0 | 0 |
| GSE166489 | HC8 | 4 | 0 | 4 | 0 | 0 |
| GSE166489 | HC9 | 25 | 0 | 25 | 0 | 0 |
| GSE167029 | HC10 | 2 | 0 | 2 | 0 | 0 |
| GSE167029 | HC11 | 17 | 0 | 17 | 0 | 0 |
| GSE167029 | HC12 | 67 | 1 | 65 | 1 | 0 |
| GSE167029 | HC13 | 8 | 0 | 5 | 3 | 0 |
| GSE167029 | HC14 | 13 | 0 | 13 | 0 | 0 |
| GSE184330 | HC1516 | 2 | 1 | 1 | 0 | 0 |
| GSE184330 | HC1718 | 2 | 0 | 2 | 0 | 0 |
| GSE167029 | HC19 | 602 | 3 | 599 | 0 | 0 |
| Ramirez- | HC2029 | 27 | 0 | 25 | 1 | 1 |
| Sanchez | | | | | | |
| GSE152450 | HC30 | 2 | 0 | 0 | 0 | 2 |
| GSE152450 | HC31 | 2 | 0 | 0 | 0 | 2 |

| | | | | | |
|---|---|---|---|---|---|
| Total | 793 | 5 | 778 | 5 | 5 |
| | (100%) | (0.6%) | (98.1%) | (0.6%) | (0.6%) |

TABLE 6
Summary of neutrophil cell counts per subpopulation across acute KD patients

| Study | | Total | Neutrophil Subpopulations | | | |
|---|---|---|---|---|---|---|
| Cohort | Dataset | Neutrophils | C0 | C1 | C2 | C3 |
| GSE168732 | KD_A7 | 1 | 1 | 0 | 0 | 0 |
| GSE168732 | KD_A8 | 6 | 0 | 5 | 1 | 0 |
| GSE168732 | KD_A9 | 4 | 0 | 4 | 0 | 0 |
| GSE168732 | KD_A10 | 421 | 339 | 72 | 10 | 0 |
| GSE168732 | KD_A11 | 54 | 35 | 15 | 4 | 0 |
| GSE168732 | KD_A12 | 681 | 625 | 49 | 7 | 0 |
| GSE167029 | KD_A13 | 31 | 0 | 27 | 4 | 0 |
| GSE167029 | KD_A14 | 21 | 1 | 20 | 0 | 0 |
| GSE167029 | KD_A15 | 64 | 4 | 57 | 3 | 0 |
| GSE183716 | KD_A16 | 8 | 1 | 6 | 1 | 0 |
| GSE152450 | KD_A17 | 51 | 0 | 2 | 0 | 49 |
| GSE152450 | KD_A18 | 2 | 0 | 1 | 0 | 1 |

| | | | | | |
|---|---|---|---|---|---|
| Total | 1344 | 1006 | 258 | 30 | 50 |
| | (100%) | (74.9%) | (19.2%) | (2.2%) | (3.7%) |

TABLE 7
Summary of neutrophil cell counts per subpopulation across acute MIS-C patients

| Study | | Total | Neutrophil Subpopulations | | | |
|---|---|---|---|---|---|---|
| Cohort | Dataset | Neutrophils | C0 | C1 | C2 | C3 |
| GSE166489 | MIS-C1 | 3914 | 3511 | 122 | 197 | 84 |
| GSE166489 | MIS-C2 | 135 | 78 | 31 | 17 | 9 |
| GSE166489 | MIS-C3 | 9 | 3 | 6 | 0 | 0 |
| GSE166489 | MIS-C4 | 112 | 1 | 99 | 7 | 5 |
| GSE166489 | MIS-C5 | 8 | 0 | 8 | 0 | 0 |
| GSE166489 | MIS-C6 | 1 | 0 | 1 | 0 | 0 |
| GSE167029 | MIS-C7 | 67 | 0 | 64 | 3 | 0 |
| GSE167029 | MIS-C8 | 28 | 2 | 18 | 2 | 6 |
| GSE167029 | MIS-C9 | 251 | 3 | 236 | 7 | 5 |
| GSE167029 | MIS-C10 | 39 | 1 | 35 | 2 | 1 |
| GSE167029 | MIS-C11 | 32 | 0 | 29 | 1 | 2 |
| GSE167029 | MIS-C12 | 17 | 0 | 17 | 0 | 0 |
| GSE167029 | MIS-C13 | 8 | 0 | 7 | 1 | 0 |
| GSE167029 | MIS-C14 | 69 | 1 | 66 | 1 | 1 |
| GSE183716 | MIS-C15 | 13 | 8 | 1 | 0 | 4 |
| GSE183716 | MIS-C16 | 1 | 0 | 1 | 0 | 0 |
| GSE184330 | MIS-C1718 | 23 | 0 | 23 | 0 | 0 |
| GSE184330 | MIS-C1920 | 4 | 0 | 4 | 0 | 0 |
| GSE184330 | MIS-C2122 | 48 | 34 | 12 | 0 | 2 |

| | | | | | |
|---|---|---|---|---|---|
| Total | 4779 | 3642 | 780 | 238 | 119 |
| | (100%) | (76.2%) | (16.3%) | (5.0%) | (2.5%) |

TABLE 8
Summary statistics of the results of the differential
abundance testing of cell types in HC vs. KD.
HC vs KD

| Cell type | logFC | logCPM | F | PValue | FDR |
|---|---|---|---|---|---|
| Neutrophils | 3.2337507 | 13.46435 | 14.0188834 | 0.0005791078 | 0.004632862 |
| B.cells | 0.7511759 | 17.25891 | 2.3154277 | 0.1360998584 | 0.389785527 |
| NK | −0.6881161 | 16.16861 | 2.1979234 | 0.1461695728 | 0.389785527 |
| Macrophages | −0.7899365 | 11.38207 | 0.9087741 | 0.3462650243 | 0.619903600 |
| T.cells | −0.3752504 | 19.02070 | 0.7638324 | 0.3874397499 | 0.619903600 |
| Monocytes | 0.3514754 | 17.75462 | 0.4736312 | 0.4953626266 | 0.660483502 |
| Plasma.cells | 0.3105306 | 10.83846 | 0.2986056 | 0.5878461881 | 0.671824215 |
| DC | 0.2753775 | 11.11295 | 0.1471754 | 0.7033164376 | 0.703316438 |

TABLE 9
Summary statistics of the results of the differential
abundance testing of cell types in HC vs. MIS-C
HC vs. MIS-C

| Cell type | logFC | logCPM | F | PValue | FDR |
|---|---|---|---|---|---|
| Neutrophils | 3.70162659 | 14.17798 | 16.792864496 | 0.000182336 | 0.001458688 |
| NK | −0.47184441 | 16.17592 | 1.553933111 | 0.219340799 | 0.860028609 |
| B.cells | 0.33224044 | 17.11078 | 1.001777626 | 0.322510728 | 0.860028609 |
| Monocytes | −0.29915419 | 17.48986 | 0.442257566 | 0.509605572 | 0.958302709 |
| Plasma.cells | 0.25410591 | 10.82209 | 0.252844206 | 0.617659392 | 0.958302709 |
| DC | 0.18468548 | 11.08139 | 0.079259180 | 0.779661714 | 0.958302709 |
| Macrophages | 0.10831148 | 11.62667 | 0.019668493 | 0.889125371 | 0.958302709 |
| T.cells | −0.01401603 | 19.13578 | 0.002765811 | 0.958302709 | 0.958302709 |

TABLE 10
Summary statistics of the results of the differential
abundance testing of neutrophil subpopulations in HC vs. KD.
HC vs. KD

| Cluster | logFC | logCPM | F | PValue | FDR |
|---|---|---|---|---|---|
| C0 | 4.6649868 | 17.31839 | 19.440465 | 0.0001087527 | 0.000435011 |
| C1 | −0.6407641 | 19.42231 | 1.685712 | 0.2034063982 | 0.405484522 |
| C2 | 0.8253954 | 15.92981 | 0.792759 | 0.3798836468 | 0.405484522 |
| C3 | 1.1856611 | 16.79292 | 0.710569 | 0.4054845221 | 0.405484522 |

TABLE 11
Summary statistics of the results of the differential
abundance testing of neutrophil subpopulations in HC vs. MIS-C.
HC vs. MIS-C

| Cluster | logFC | logCPM | F | PValue | FDR |
|---|---|---|---|---|---|
| C0 | 4.3977683 | 17.09088 | 14.9519144 | 0.0003843652 | 0.001537461 |
| C1 | 0.7874335 | 15.51830 | 0.9614167 | 0.3325527593 | 0.665105519 |
| C2 | −0.2135969 | 19.57356 | 0.3473958 | 0.5588092073 | 0.682318358 |
| C3 | 0.4206928 | 15.79155 | 0.1699216 | 0.6823183583 | 0.682318358 |

### Example 4

### Hyperactivated CD177^{±} Neutrophils Acquire Enhanced Neutrophil Effector Functions

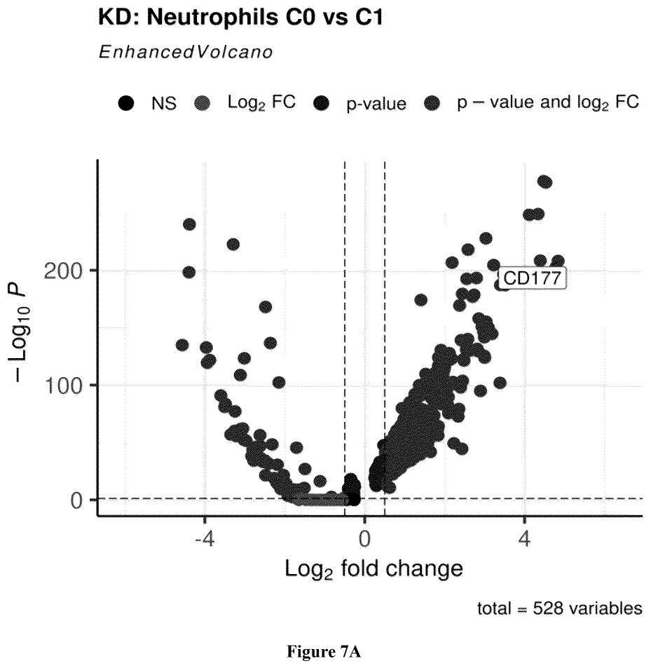

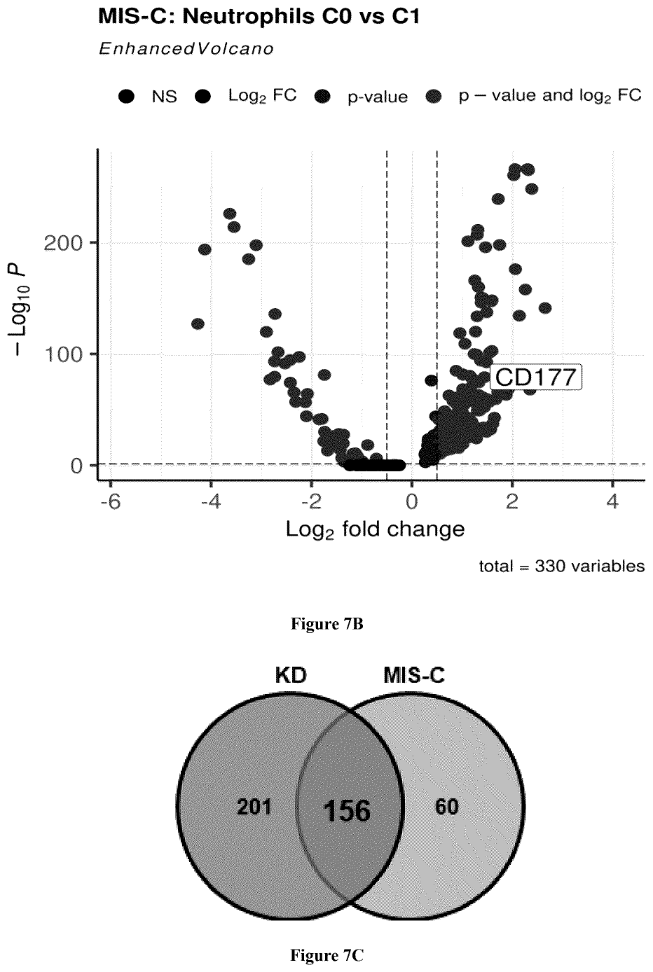

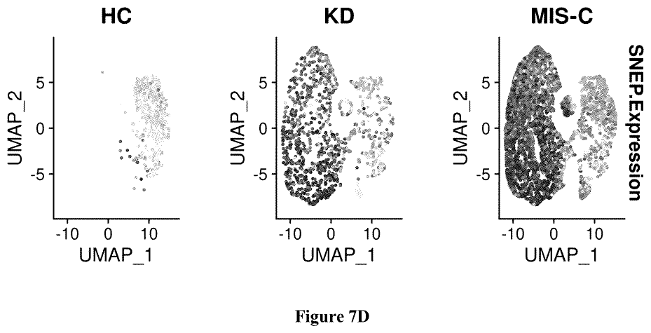

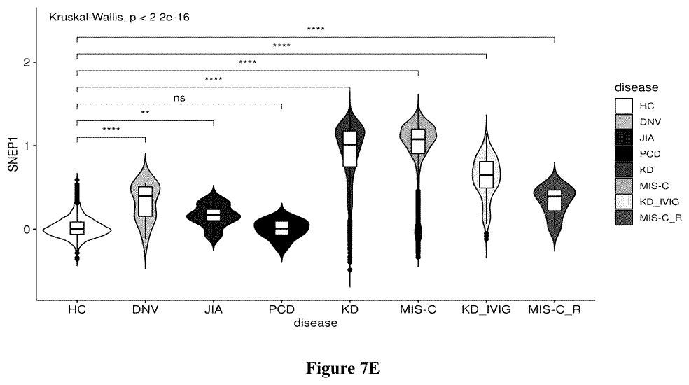

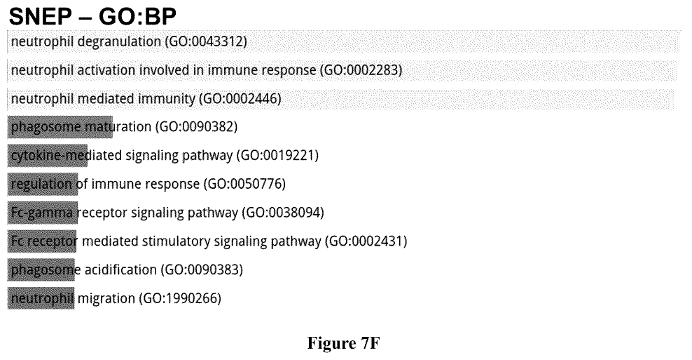

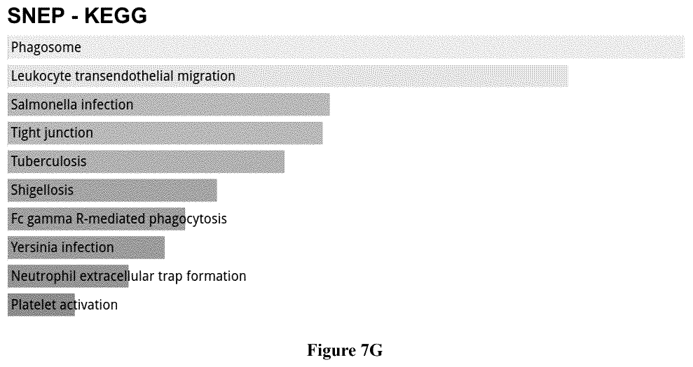

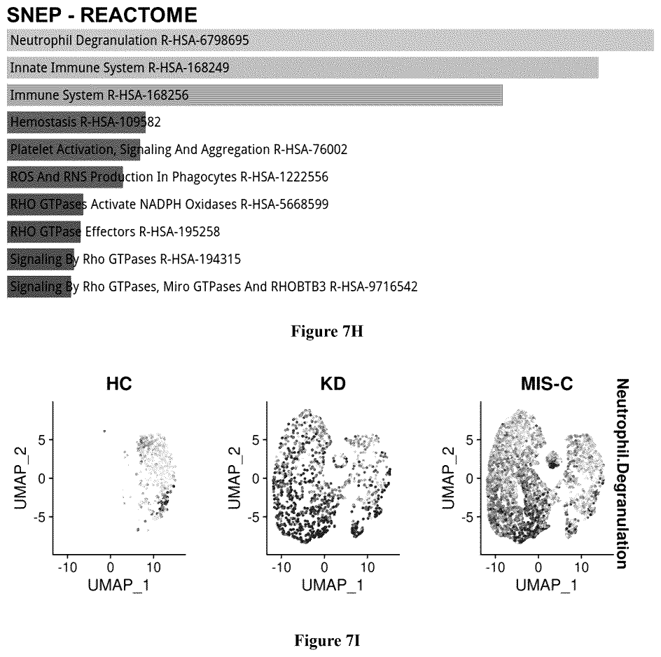

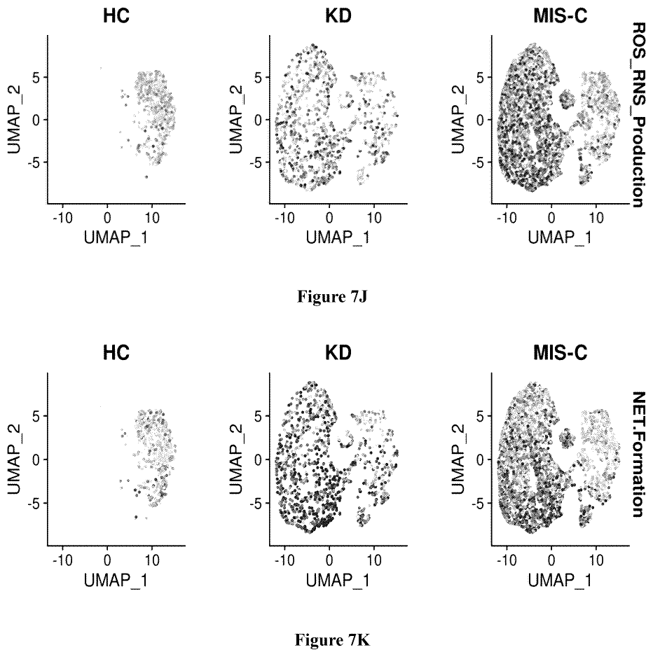

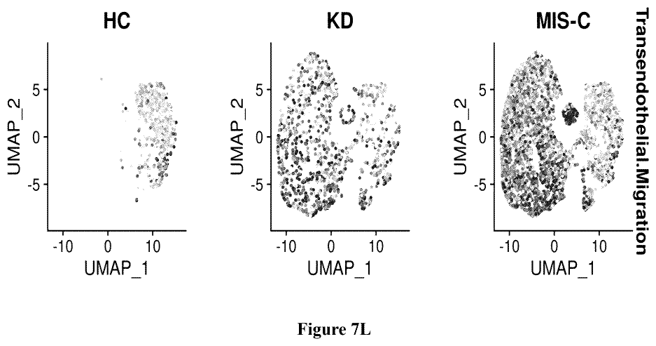

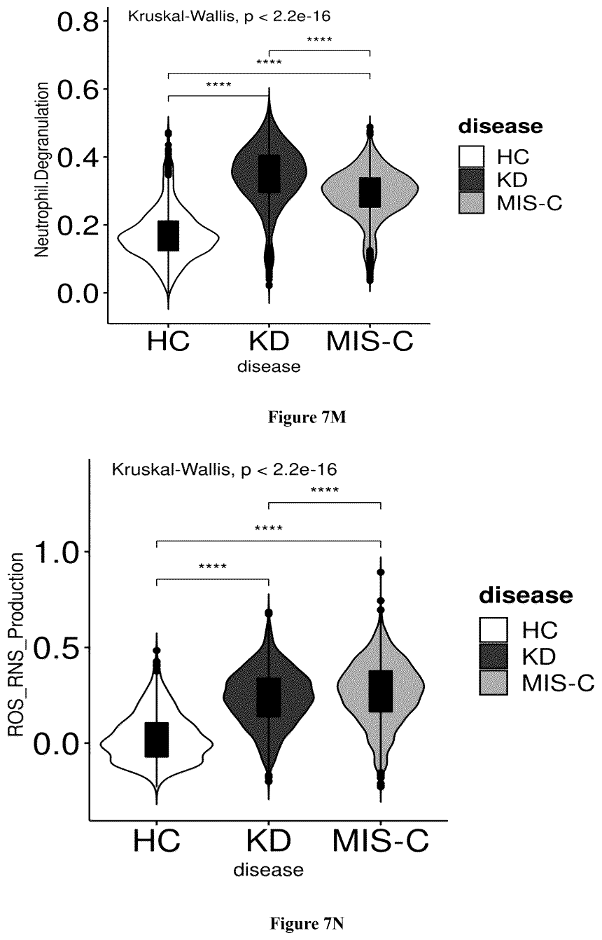

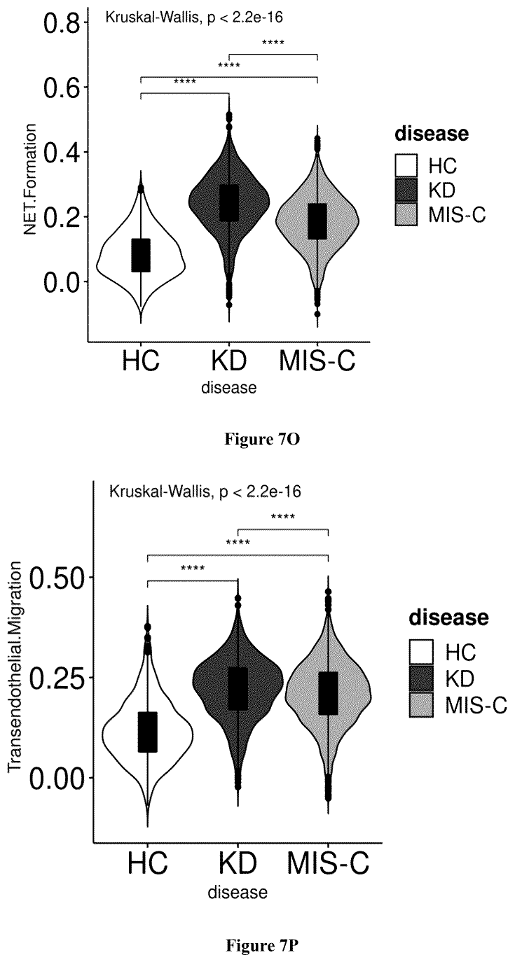

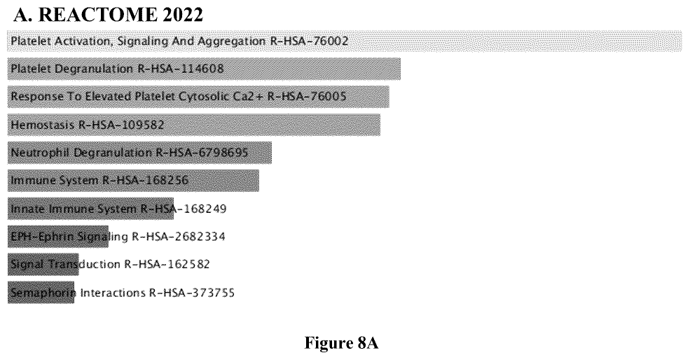

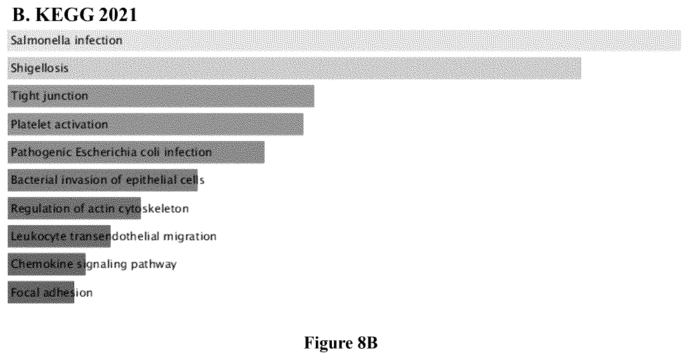

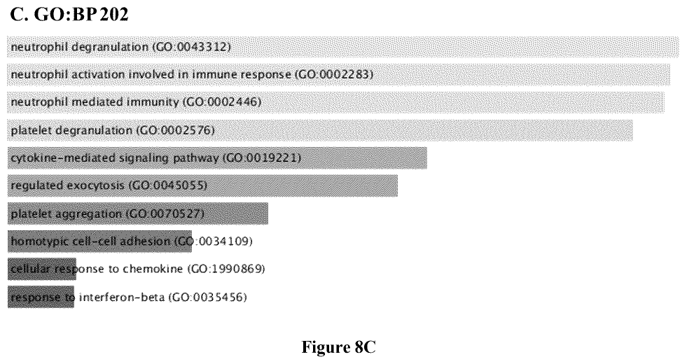

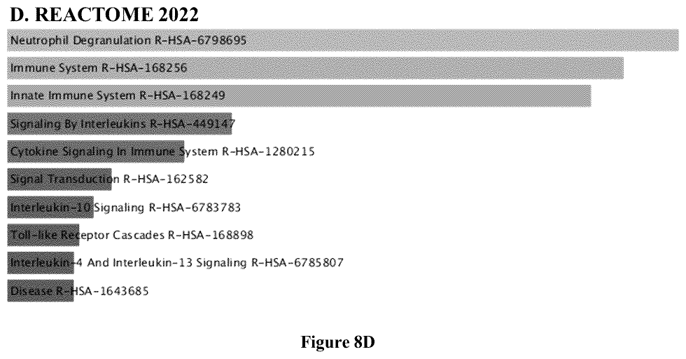

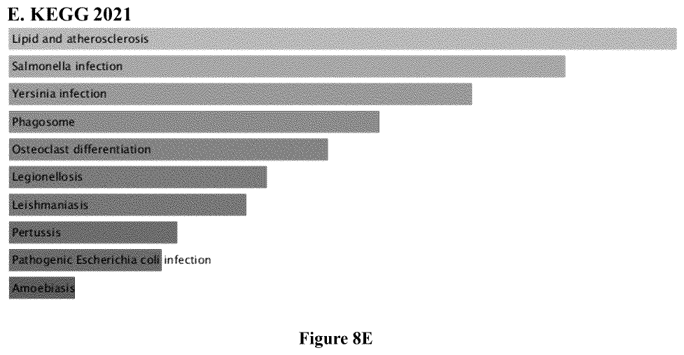

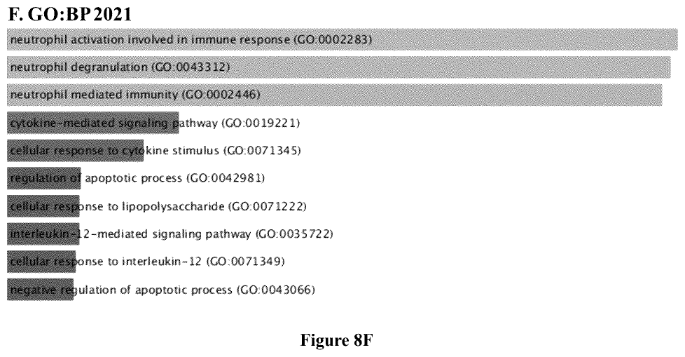

Because the CD177⁺ neutrophils of KD and MIS-C are highly similar (r-0.95; FIG. 6B), we investigated whether this contributes to their shared immunopathogenesis. To characterize the molecular mechanisms underlying neutrophil hyperactivation and its shared pathological role in KD and MIS-C, we examined the dysregulated genes in KD and MIS-C. We performed differential expression analysis in cluster 0 (C0) versus cluster 1 (C1; FIGS. 7A and 7B) and obtained the shared neutrophil expression program (SNEP) on the basis of the upregulated differentially expressed gene shared by KD and MIS-C(FIG. 7C; Table 12). We defined the SNEP as the upregulated genes found in the hyperactivated neutrophil subpopulation in both KD and MIS-C, the expression of which is associated with acute stages, IVIG treatment, severity, and recovery. The SNEP in KD and MIS-C could facilitate the identification of key targets that may be used in the development of a single therapeutic strategy against both KD and MIS-C by targeting a common pathological regulator. We identified 357 upregulated genes in KD-neutrophil C0 and 216 upregulated genes in MIS-C-neutrophil C0 (FIG. 7C), in which the SNEP of KD and MIS-C was composed of 156 genes. We found that genes involved in neutrophil processes and cardiovascular complications were markedly upregulated, as well as genes that have unknown roles in neutrophil functioning. Consistent with other studies, proinflammatory damage-associated molecular pattern molecules and the calcium-binding S100 protein family (S100A6/8/9/11/P), known to play a key role in inflammatory and cardiovascular diseases, were also overexpressed in both diseases.

TABLE 12
The 156 upregulated DEGs comprising the shared neutrophil
expression programs (SNEP) ofKD and MIS-C

| | | | | | |
|---|---|---|---|---|---|
| ABTB1 | C1orf162 | FKBP5 | IL17RA | OAZ1 | S100A6 |
| ACTB | C4orf3 | FLOT1 | IL1R2 | OSTF1 | S100A8 |
| ACTN1 | C4orf48 | FOLR3 | ITGB2 | PFN1 | S100A9 |
| ADGRE5 | CAP1 | FPR1 | ITM2B | PGD | S100P |
| AGTRAP | CARD16 | FYB1 | JPT1 | PGS1 | SDCBP |
| AL445524.1 | CD177 | GABARAP | LAMTOR4 | PLP2 | SELL |
| ALDOA | CDA | GABARAPL2 | LILRA5 | PRDX5 | SELPLG |
| ALOX5AP | CFL1 | GCA | LILRB3 | PRR13 | SERPINA1 |
| ALPL | CFLAR | GLIPR2 | LSP1 | PSMB3 | SERPINB1 |
| ANP32A | CHMP2A | GLRX | LST1 | PSMB9 | SMIM25 |
| ANXA3 | CKLF | GMFG | LY96 | PYCARD | SOD2 |
| APBB1IP | CLIC1 | GNB2 | MCEMP1 | PYGL | SORL1 |
| AQP9 | CMTM2 | GNG5 | MMP25 | R3HDM4 | STXBP2 |
| ARG1 | CNN2 | GPSM3 | MMP9 | RAB31 | TAGLN2 |
| ARHGAP9 | CORO1A | GRINA | MNDA | RAB5IF | TALDO1 |
| ARHGDIB | CPPED1 | GYG1 | MSRB1 | RAB7A | TRAPPC1 |
| ARPC5 | CST7 | H3F3A | MYL12A | RAC2 | TSPO |
| ATP6V0B | CSTB | HCK | MYL12B | RAP1A | TUBA4A |
| ATP6V0C | CYSTM1 | HCLS1 | MYL6 | RGS19 | TXN |
| ATP6V0D1 | DYSF | HMGB2 | NAIP | RHOA | TYROBP |
| ATP6V0E1 | FAM129A | HRH2 | NCF1 | RHOG | UBL5 |
| BASP1 | FCER1G | ICAM3 | NCF4 | RIPOR2 | USP10 |
| BCL2A1 | FCGR2A | IFITM1 | NDUFB3 | RNASET2 | VNN2 |
| BCL6 | FCGR3B | IFITM2 | NEAT1 | RTN3 | VSIR |
| BLOC1S1 | FGR | IFITM3 | NFE2 | S100A11 | WAS |
| C19orf38 | FKBP1A | IGSF6 | NOP10 | S100A12 | ZYX |

The SNEP signature was highly activated in neutrophil C0 (FIG. 7D). Across all diseases, the SNEP was significantly overexpressed in KD and MIS-C and was associated with KD IVIG treatment and MIS-C recovery (FIG. 7E), which suggests a prognostic connection between KD and MIS-C. To understand the effect of the aberrant enrichment of the genes shared by KD and MIS-C neutrophils, we performed a comprehensive pathway analysis using the REACTOME, KEGG, and GO: BP databases (FIG. 7F˜7H; Tables 13˜15). The significantly enriched pathways were related to CD177⁺ neutrophil effector functions, including neutrophil degranulation (adjusted P<1.62e-32), ROS/RNS (reactive oxygen species/reactive nitrogen species) production (adjusted P<9.29e-7), leukocyte transendothelial migration (adjusted P<1.15e-7), and NET formation (adjusted P<4.12e-5).

TABLE 13
SNEP top 10 REACTOME pathways

| | | | Adj. | Odds | Combined | |
|---|---|---|---|---|---|---|
| Term | Overlap | P-value | P- value | Ratio | Score | Genes |
| Neutrophil | 44/468 | 3.08E−35 | 1.62E−32 | 17.99 | 1429.88 | ARHGAP9; CDA; CSTB; |
| Degranulation | | | | | | SERPINA1; GMFG; ITGB2; |
| R-HSA-6798695 | | | | | | FPR1; PYGL; PYCARD; |
| | | | | | | CNN2; SDCBP; PRDX5; |
| | | | | | | RAP1A; ADGRE5; S100A12; |
| | | | | | | OSTF1; GYG1; S100A11; |
| | | | | | | CD177; CAP1; SERPINB1; |
| | | | | | | TRAPPC1; FCER1G; GCA; |
| | | | | | | RNASET2; RHOG; MCEMP1; |
| | | | | | | ARPC5; MMP9; RHOA; |
| | | | | | | CPPED1; FGR; RAB31; |
| | | | | | | TYROBP; FCGR2A; SELL; |
| | | | | | | CYSTM1; MNDA; S100P; |
| | | | | | | FOLR3; S100A9; ATP6V0C; |
| | | | | | | S100A8; RAB7A |
| Innate | 56/1035 | 1.80E−32 | 4.73E−30 | 10.79 | 788.79 | ARHGAP9; CDA; SERPINA1; |
| Immune | | | | | | NCF1; GMFG; NCF4; |
| System | | | | | | ITGB2; ICAM3; PYGL; |
| R-HSA-168249 | | | | | | PYCARD; ADGRE5; RAC2; |
| | | | | | | OSTF1; GYG1; CD177; |
| | | | | | | CAP1; ATP6V0B; SERPINB1; |
| | | | | | | ATP6V0E1; FCER1G; RNASET2; |
| | | | | | | RHOG; MCEMP1; MMP9; |
| | | | | | | RHOA; FGR; HCK; |
| | | | | | | RAB31; TYROBP; ATP6V0D1; |
| | | | | | | S100A9; ATP6V0C; S100A8; |
| | | | | | | RAB7A; CSTB; FPR1; |
| | | | | | | WAS; LY96; CNN2; |
| | | | | | | SDCBP; PRDX5; RAP1A; |
| | | | | | | PSMB3; S100A12; S100A11; |
| | | | | | | TRAPPC1; GCA; ARPC5; |
| | | | | | | CPPED1; PSMB9; FCGR2A; |
| | | | | | | SELL; CYSTM1; MNDA; |
| | | | | | | S100P; FOLR3 |
| Immune | 67/1943 | 1.08E−27 | 1.90E−25 | 7.21 | 447.70 | IFITM3; ARHGAP9; CDA; |
| System | | | | | | IFITM1; IFITM2; SERPINA1; |
| R-HSA-168256 | | | | | | NCF1; GMFG; NCF4; |
| | | | | | | ITGB2; ICAM3; PYGL; |
| | | | | | | PYCARD; ADGRE5; RAC2; |
| | | | | | | OSTF1; GYG1; CD177; |
| | | | | | | CAP1; ATP6V0B; SERPINB1; |
| | | | | | | ATP6V0E1; FCER1G; IL1R2; |
| | | | | | | RNASET2; RHOG; TALDO1; |
| | | | | | | MCEMP1; MMP9; RHOA; |
| | | | | | | IL17RA; FGR; HCK; |
| | | | | | | RAB31; TYROBP; ATP6V0D1; |
| | | | | | | S100A9; ATP6V0C; S100A8; |
| | | | | | | RAB7A; CSTB; STXBP2; |
| | | | | | | FPR1; WAS; LY96; |
| | | | | | | LILRA5; CNN2; SDCBP; |
| | | | | | | PRDX5; RAP1A; PSMB3; |
| | | | | | | S100A12; S100A11; FYB1; |
| | | | | | | TRAPPC1; GCA; ARPC5; |
| | | | | | | CPPED1; PSMB9; FKBP1A; |
| | | | | | | FCGR2A; BCL6; SELL; |
| | | | | | | CYSTM1; MNDA; S100P; |
| | | | | | | FOLR3 |
| Hemostasis | 22/576 | 7.85E−10 | 1.03E−7 | 5.72 | 119.85 | CAP1; NFE2; SERPINA1; |
| R-HSA-109582 | | | | | | FCER1G; SELPLG; ACTN1; |
| | | | | | | STXBP2; ITGB2; RHOG; |
| | | | | | | TUBA4A; RHOA; APBB1IP; |
| | | | | | | FGR; RAP1A; SELL; GNG5; |
| | | | | | | GNB2; RAC2; TAGLN2; |
| | | | | | | PFN1; S100A9; CD177 |
| Platelet | 15/254 | 1.46E−9 | 1.54E−7 | 8.73 | 177.54 | CAP1; SERPINA1; FCER1G; |
| Activation, | | | | | | ACTN1; STXBP2; RHOG; |
| Signaling And | | | | | | TUBA4A; RHOA; APBB1IP; |
| Aggregation | | | | | | RAP1A; GNG5; GNB2; |
| RHSA-76002 | | | | | | RAC2; TAGLN2; PFN1 |
| ROS And RNS | 7/36 | 1.06E−8 | 9.29E−7 | 32.10 | 589.46 | ATP6V0B; ATP6V0E1; NCF1; |
| Production In | | | | | | NCF4; RAC2; ATP6V0D1; |
| Phagocytes | | | | | | ATP6V0C |
| R-HSA-1222556 | | | | | | |
| RHO GTPases | 5/24 | 1.02E−6 | 7.67E−5 | 34.55 | 476.62 | NCF1; NCF4; RAC2; |
| Activate | | | | | | S100A9; S100A8 |
| NADPH | | | | | | |
| Oxidases | | | | | | |
| R-HSA-5668599 | | | | | | |
| RHO GTPase | 12/269 | 1.39E−6 | 9.11E−5 | 6.35 | 85.67 | MYL6; NCF1; NCF4; RAC2; |
| Effectors | | | | | | RHOG; WAS; ARPC5; |
| R-HSA-195258 | | | | | | PFN1; S100A9; S100A8; |
| | | | | | | RHOA; MYL12B |
| Signaling | 18/644 | 2.94E−6 | 1.72E−4 | 4.00 | 51.00 | ARHGAP9; NCF1; ACTN1; |
| By Rho | | | | | | NCF4; RHOG; WAS; |
| GTPases | | | | | | ARPC5; RHOA; MYL12B; |
| R-HSA-194315 | | | | | | MYL6; BASP1; ARHGDIB; |
| | | | | | | FLOT1; RAC2; PFN1; |
| | | | | | | S100A9; S100A8; RAB7A |
| Signaling By | 18/660 | 4.13E−6 | 2.17E−4 | 3.90 | 48.36 | ARHGAP9; NCF1; ACTN1; |
| Rho GTPases, | | | | | | NCF4; RHOG; WAS; |
| Miro GTPases | | | | | | ARPC5; RHOA; MYL12B; |
| And RHOBTB3 | | | | | | MYL6; BASP1; ARHGDIB; |
| R-HSA-9716542 | | | | | | FLOT1; RAC2; PFN1; |
| | | | | | | S100A9; S100A8; RAB7A |

TABLE 14
SNEP top 10 KEGG pathways

| | | | Adj. | Odds | Combined | |
|---|---|---|---|---|---|---|
| Term | Overlap | P-value | P- value | Ratio | Score | Genes |
| Phagosome | 13/152 | 2.06E−10 | 3.21E−8 | 12.89 | 287.44 | ATP6V0B; ATP6V0E1; |
| | | | | | | NCF1; NCF4; ITGB2; |
| | | | | | | CORO1A; ACTB; TUBA4A; |
| | | | | | | FCGR3B; FCGR2A; ATP6V0D1; |
| | | | | | | ATP6V0C; RAB7A |
| Leukocyte | 11/114 | 1.47E−9 | 1.15E−7 | 14.54 | 295.70 | RAP1A; NCF1; ACTN1; |
| transendothelial | | | | | | NCF4; ITGB2; RAC2; |
| migration | | | | | | MMP9; ACTB; MYL12A; |
| | | | | | | RHOA; MYL12B |
| Salmonella | 13/249 | 8.12E−8 | 3.57E−6 | 7.55 | 123.31 | RHOG; LY96; TXN; ARPC5; |
| infection | | | | | | ACTB; TUBA4A; MYL12A; |
| | | | | | | RHOA; MYL12B; PYCARD; |
| | | | | | | PFN1; NAIP; RAB7A |
| Tight | 11/169 | 9.14E−8 | 3.57E−6 | 9.45 | 153.20 | MYL6; RAP1A; ACTN1; |
| junction | | | | | | WAS; HCLS1; ARPC5; |
| | | | | | | ACTB; TUBA4A; MYL12A; |
| | | | | | | RHOA; MYL12B |
| Tuberculosis | 11/180 | 1.74E−7 | 5.42E−6 | 8.83 | 137.48 | ATP6V0B; FCGR3B; FCER1G; |
| | | | | | | FCGR2A; ITGB2; LSP1; |
| | | | | | | ATP6V0D1; CORO1A; |
| | | | | | | ATP6V0C; RHOA; RAB7A |
| Shigellosis | 12/246 | 5.40E−7 | 1.40E−5 | 6.99 | 100.79 | PYCARD; GABARAPL2; |
| | | | | | | ACTN1; HCLS1; ARPC5; |
| | | | | | | PFN1; ACTB; NAIP; |
| | | | | | | MYL12A; GABARAP; |
| | | | | | | RHOA; MYL12B |
| Fc gamma R- | 8/97 | 9.19E−7 | 2.05E−5 | 12.00 | 166.78 | HCK; FCGR3B; FCGR2A; |
| mediated | | | | | | NCF1; CFL1; RAC2; WAS; |
| phagocytosis | | | | | | ARPC5 |
| Yersinia | 9/137 | 1.30E−6 | 2.53E−5 | 9.43 | 127.83 | PYCARD; FYB1; FCGR2A; |
| infection | | | | | | RAC2; RHOG; WAS; |
| | | | | | | ARPC5; ACTB; RHOA |
| Neutrophil | 10/189 | 2.38E−6 | 4.12E−5 | 7.52 | 97.44 | FCGR3B; SELPLG; |
| extracellular | | | | | | FCGR2A; NCF1; NCF4; |
| trap formation | | | | | | AQP9; ITGB2; FPR1; |
| | | | | | | RAC2; ACTB |
| Platelet | 8/124 | 5.86E−6 | 9.14E−5 | 9.20 | 110.75 | APBB1IP; RAP1A; |
| activation | | | | | | FCER1G; FCGR2A; ACTB; |
| | | | | | | MYL12A; RHOA; MYL12B |

TABLE 15
SNEP top 10 GO: BP pathways

| | | | Adj. | Odds | Combined | |
|---|---|---|---|---|---|---|
| Term | Overlap | P-value | P- value | Ratio | Score | Genes |
| Neutrophil | 50/481 | 1.90E−42 | 1.84E−39 | 21.25 | 2041.09 | ARHGAP9; CDA; |
| degranulation | | | | | | SERPINA1; GMFG; ITGB2; |
| (GO: 0043312) | | | | | | PYGL; PYCARD; MMP25; |
| | | | | | | FCGR3B; ADGRE5; OSTF1; |
| | | | | | | GYG1; CD177; CAP1; |
| | | | | | | SERPINB1; FCER1G; |
| | | | | | | ARG1; ANXA3; RNASET2; |
| | | | | | | RHOG; MCEMP1; MMP9; |
| | | | | | | RHOA; FGR; RAB31; |
| | | | | | | TYROBP; ALDOA; S100A9; |
| | | | | | | ATP6V0C; S100A8; |
| | | | | | | RAB7A; CSTB; STXBP2; |
| | | | | | | FPR1; CNN2; SDCBP; |
| | | | | | | RAP1A; S100A12; S100A11; |
| | | | | | | TRAPPC1; GCA; ARPC5; |
| | | | | | | LILRB3; CPPED1; |
| | | | | | | FCGR2A; SELL; CYSTM1; |
| | | | | | | S100P; MNDA; FOLR3 |
| Neutrophil | 50/485 | 2.87E−42 | 1.84E−39 | 21.05 | 2013.17 | ARHGAP9; CDA; |
| activation | | | | | | SERPINA1; GMFG; ITGB2; |
| involved in | | | | | | PYGL; PYCARD; MMP25; |
| immune | | | | | | FCGR3B; ADGRE5; OSTF1; |
| response | | | | | | GYG1; CD177; CAP1; |
| (GO: 0002283) | | | | | | SERPINB1; FCER1G; |
| | | | | | | ARG1; ANXA3; RNASET2; |
| | | | | | | RHOG; MCEMP1; MMP9; |
| | | | | | | RHOA; FGR; RAB31; |
| | | | | | | TYROBP; ALDOA; S100A9; |
| | | | | | | ATP6V0C; S100A8; RAB7A; |
| | | | | | | CSTB; STXBP2; FPR1; |
| | | | | | | CNN2; SDCBP; RAP1A; |
| | | | | | | S100A12; S100A11; |
| | | | | | | TRAPPC1; GCA; ARPC5; |
| | | | | | | LILRB3; CPPED1; FCGR2A; |
| | | | | | | SELL; CYSTM1; S100P; |
| | | | | | | MNDA; FOLR3 |
| Neutrophil | 50/488 | 3.91E−42 | 1.84E−39 | 20.90 | 1992.61 | ARHGAP9; CDA; |
| mediated | | | | | | SERPINA1; GMFG; ITGB2; |
| immunity | | | | | | PYGL; PYCARD; MMP25; |
| (GO: 0002446) | | | | | | FCGR3B; ADGRE5; OSTF1; |
| | | | | | | GYG1; CD177; CAP1; |
| | | | | | | SERPINB1; FCER1G; |
| | | | | | | ARG1; ANXA3; RNASET2; |
| | | | | | | RHOG; MCEMP1; MMP9; |
| | | | | | | RHOA; FGR; RAB31; |
| | | | | | | TYROBP; ALDOA; S100A9; |
| | | | | | | ATP6V0C; S100A8; |
| | | | | | | RAB7A; CSTB; STXBP2; |
| | | | | | | FPR1; CNN2; SDCBP; |
| | | | | | | RAP1A; S100A12; S100A11; |
| | | | | | | TRAPPC1; GCA; ARPC5; |
| | | | | | | LILRB3; CPPED1; |
| | | | | | | FCGR2A; SELL; CYSTM1; |
| | | | | | | S100P; MNDA; FOLR3 |
| Phagosome | 7/37 | 1.30E−8 | 4.59E−6 | 31.03 | 563.48 | ATP6V0B; ATP6V0E1; |
| maturation | | | | | | RAB31; ATP6V0D1; |
| (GO: 0090382) | | | | | | CORO1A; ATP6V0C; |
| | | | | | | RAB7A |
| Cytokine- | 19/621 | 3.95E−7 | 1.12E−4 | 4.43 | 65.36 | IFITM3; IFITM1; IFITM2; |
| mediated | | | | | | FCER1G; IL1R2; ITGB2; |
| signaling | | | | | | FPR1; TALDO1; SOD2; |
| pathway | | | | | | MMP9; IL17RA; PSMB9; |
| (GO: 0019221) | | | | | | PYCARD; CNN2; HCK; |
| | | | | | | BCL6; PSMB3; CFL1; PLP2 |
| Regulation of | 10/179 | 1.46E−6 | 2.99E−4 | 7.97 | 107.17 | FGR; FKBP1A; IFITM1; |
| immune | | | | | | TYROBP; FCGR3B; |
| response | | | | | | FCGR2A; BCL6; SELL; |
| (GO: 0050776) | | | | | | ITGB2; ICAM3 |
| Fc-gamma | 7/72 | 1.48E−6 | 2.99E−4 | 14.30 | 191.91 | FGR; HCK; FCER1G; |
| receptor | | | | | | FCGR2A; WAS; ARPC5; |
| signaling | | | | | | ACTB |
| pathway | | | | | | |
| (GO: 0038094) | | | | | | |
| Fc receptor | 7/74 | 1.78E−6 | 3.15E−4 | 13.87 | 183.57 | FGR; HCK; FCER1G; |
| mediated | | | | | | FCGR2A; WAS; ARPC5; |
| stimulatory | | | | | | ACTB |
| signaling | | | | | | |
| pathway | | | | | | |
| (GO: 0002431) | | | | | | |
| Phagosome | 5/28 | 2.30E−6 | 3.30E−4 | 28.54 | 370.45 | ATP6V0B; ATP6V0E1; |
| acidification | | | | | | ATP6V0D1; ATP6V0C; |
| (GO: 0090383) | | | | | | RAB7A |
| Neutrophil | 7/77 | 2.34E−6 | 3.30E−4 | 13.27 | 172.09 | FCER1G; ITGB2; S100A12; |
| migration | | | | | | S100A9; CKLF; S100A8; |
| (GO: 1990266) | | | | | | CD177 |

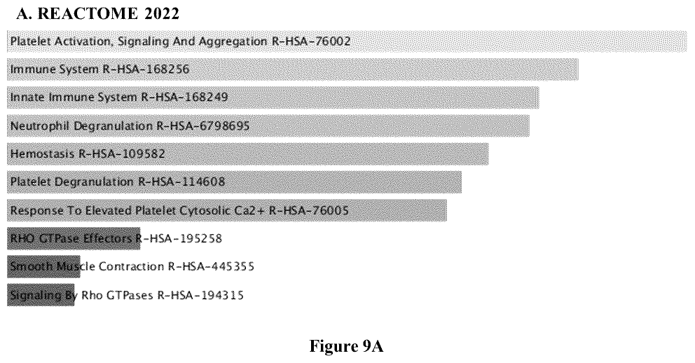

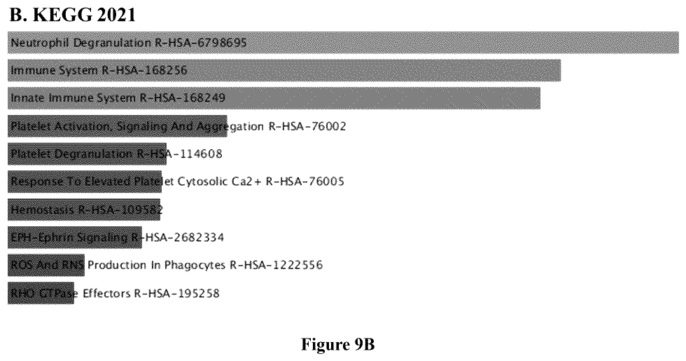

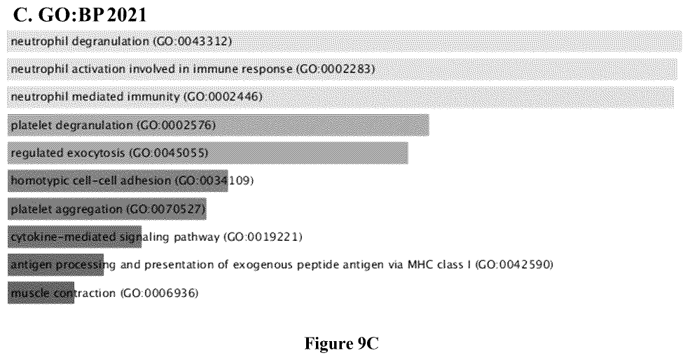

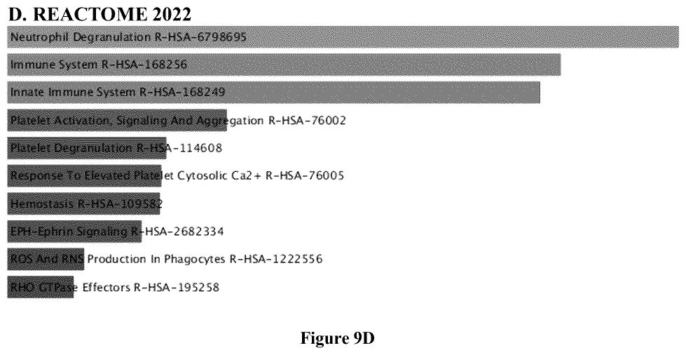

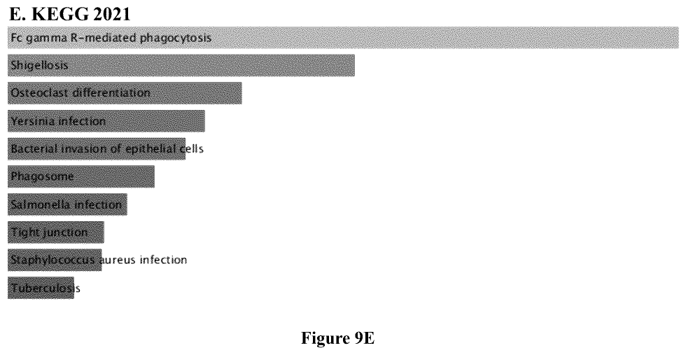

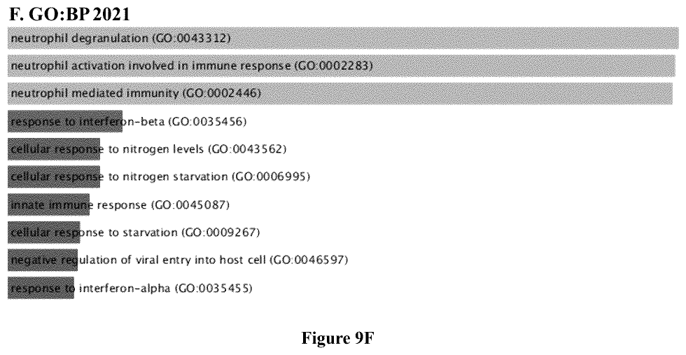

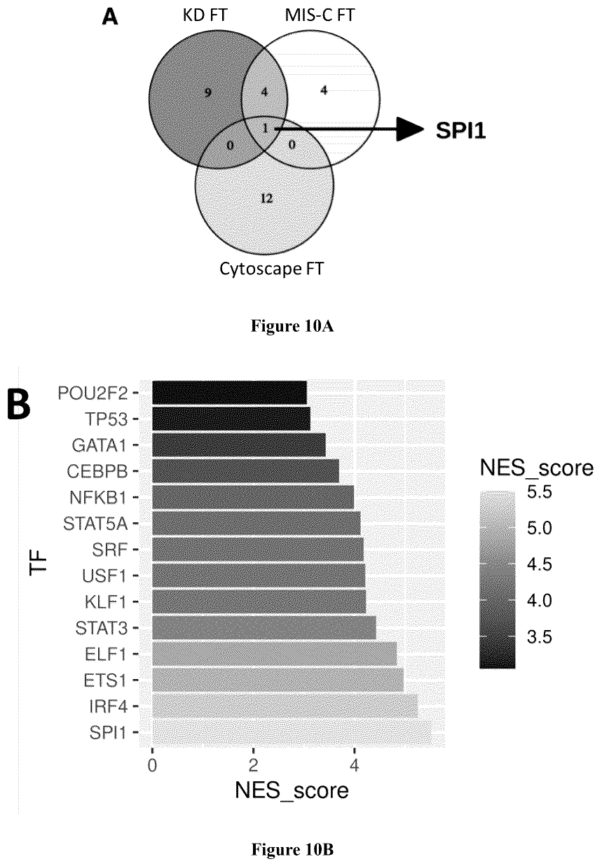

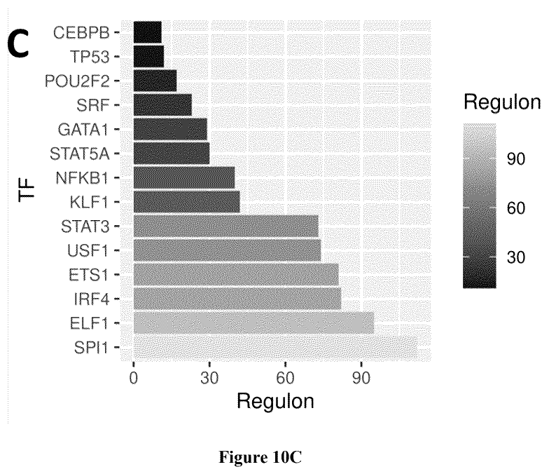

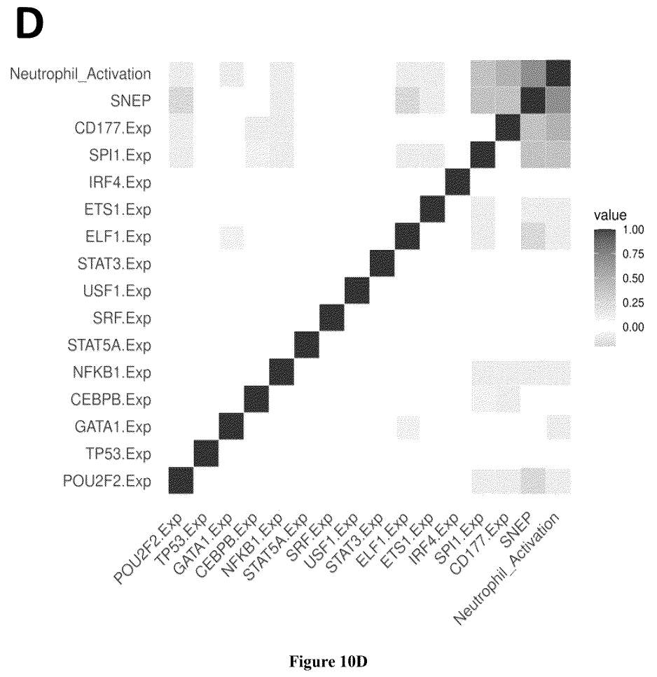

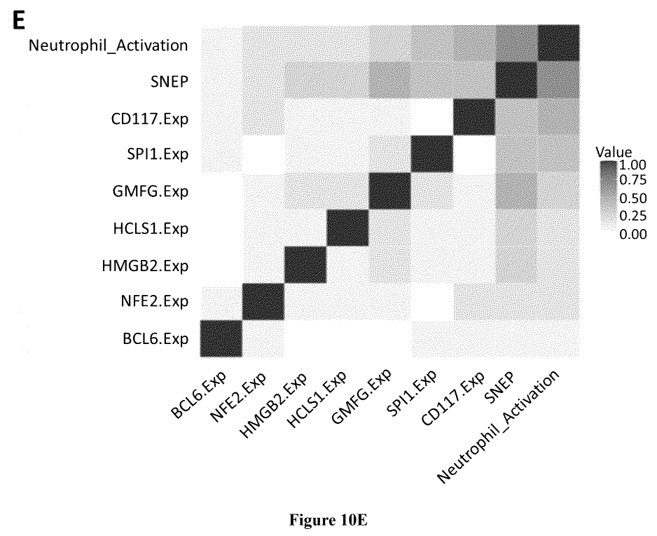

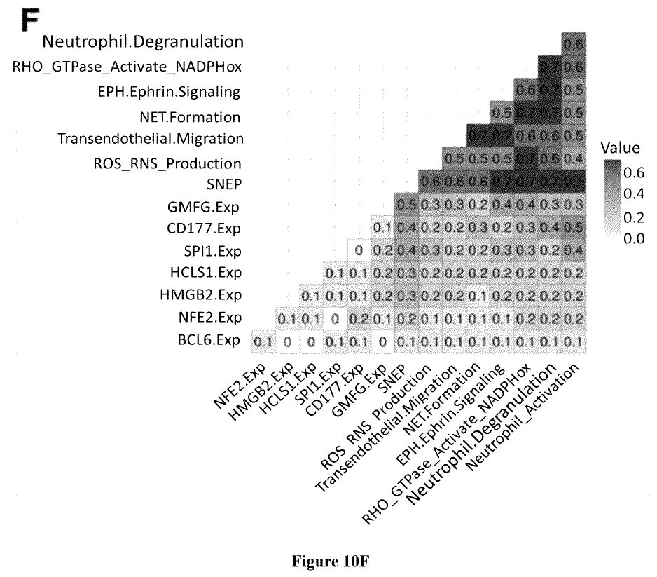

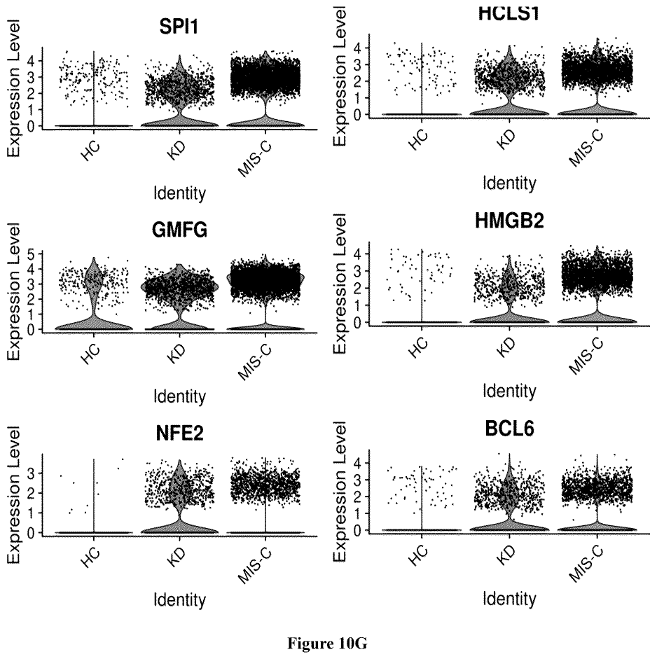

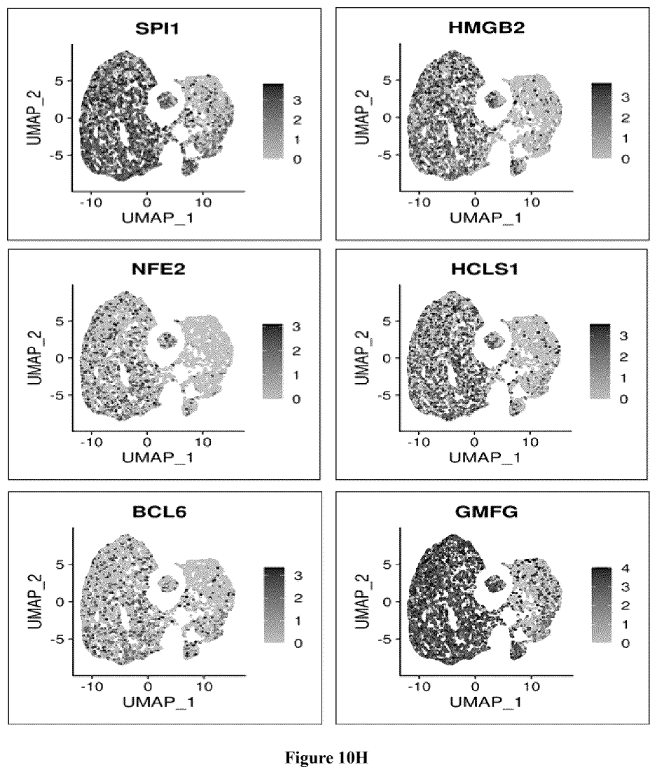

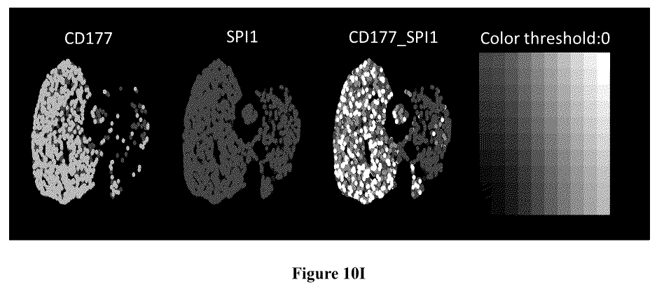

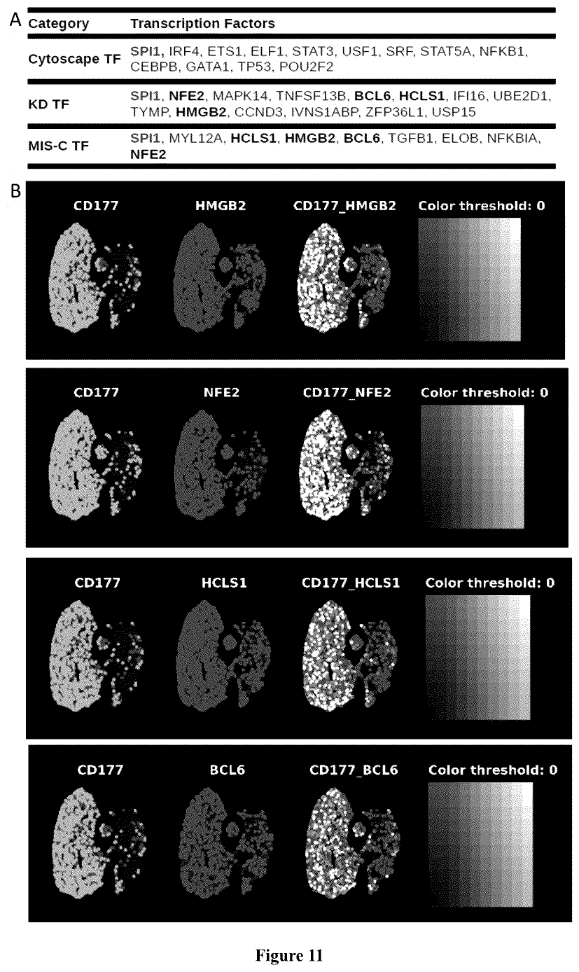

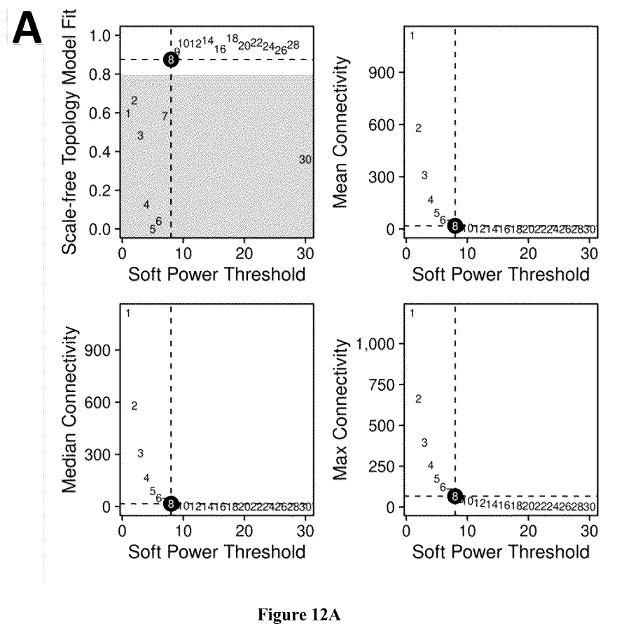

These effector processes showed significantly higher expression in the KD and MIS-C groups than in the HC group (P<2.23e-16; FIG. 7I˜7P). Expression levels of these processes are aberrantly high in KD and MIS-C compared with homeostasis and other pediatric diseases. On the other hand, neutrophil clusters 2 and 3 also had similar pathways in general (FIGS. 9 and 10); however, only cluster 0 has CD177 hyperexpression (FIG. 5D) and significant differential abundance (Tables 10 and 11). Based on these findings, the overactivation of effector pathways in CD177⁺ neutrophils support its potential pathogenic role in both diseases.

### Example 5

### Potential Master Regulator of the Elevated Transcriptional Programs During Neutrophil Hyperactivation

The activation of transcriptional programs indicates an upregulated transcription of certain genes, driving a phenotype that is shaped by TFs.40 Therefore, we then sought to identify the master regulators or TFs of the upregulated genes that could be required in mediating pathological damages driven by CD177 neutrophils. Cytoscape iRegulon analysis determined 13 TFs (SPI1 [Spi-1 protooncogene], IRF4, ETS1, ELF1, STAT3, USF1, SRF, STAT5A, NFKB1, CEBPB, GATA1, TP53, and POU2F2) that may regulate the SNEP with high normalized enrichment score (>3.0; FIGS. 10A and 10B), but only SPI1 is upregulated during KD or MIS-C (FIG. 11A). Meanwhile, we identified 14 KD TFs and 9 MIS-C TFs upregulated in SNEP (FIG. 11A), where 5 TFs are correlated with neutrophil activation and effector pathways. These TFs are highly upregulated in cluster 0 of both KD and MISC (SPI1, HCLS1, HMGB2, BCL6, and NFE2; FIGS. 10G and 10H) and are coexpressed with CD177 (FIG. 11B).

Our data highlighted SPI1 as the primary candidate master transcription regulator with the highest regulon size (107/156; FIG. 10C). Comparing Cytoscape TF, KD TF, and MIS-C TF, SPI1 was identified as a strong candidate (FIG. 10A). Only SPI1 is positively correlated with neutrophil activation and neutrophil effector pathways (FIG. 10D˜10F). Coexpression analysis further revealed the high SPIL expression of CD177 neutrophils (FIG. 10I).

Meanwhile, we also noticed the enhanced expression of a growth factor, GMFG (Glia Maturation Factor Gamma; FIGS. 10E, 10F, 10G, and 10H), initially identified as a probrain cell differentiation factor. Later evidence pointed out that GMFG also promotes neutrophil chemotaxis. In our TF correlation analysis, we found the positive correlation of GMFG with the expression of the 5 TFs of KD and MIS-C, as well as the expression of CD177, SPI1, and SNEP, and neutrophil activation. Hence, in the context of KD and MIS-C, the growth factor GMFG may also be relevant to CD177⁺ neutrophil biology that further experiments, especially on neutrophil proliferation and expansion, may confirm in the future.

We postulate that these TFs are potential transcriptional regulatory genes supporting elevated neutrophil activation and effector functions and our analysis highlights SPI1's potential major activity during KD and MIS-C.

### Example 6

### High-Dimensional Weighted Gene Coexpression Network Analysis to Identify Critical Modulators of Neutrophil Activation

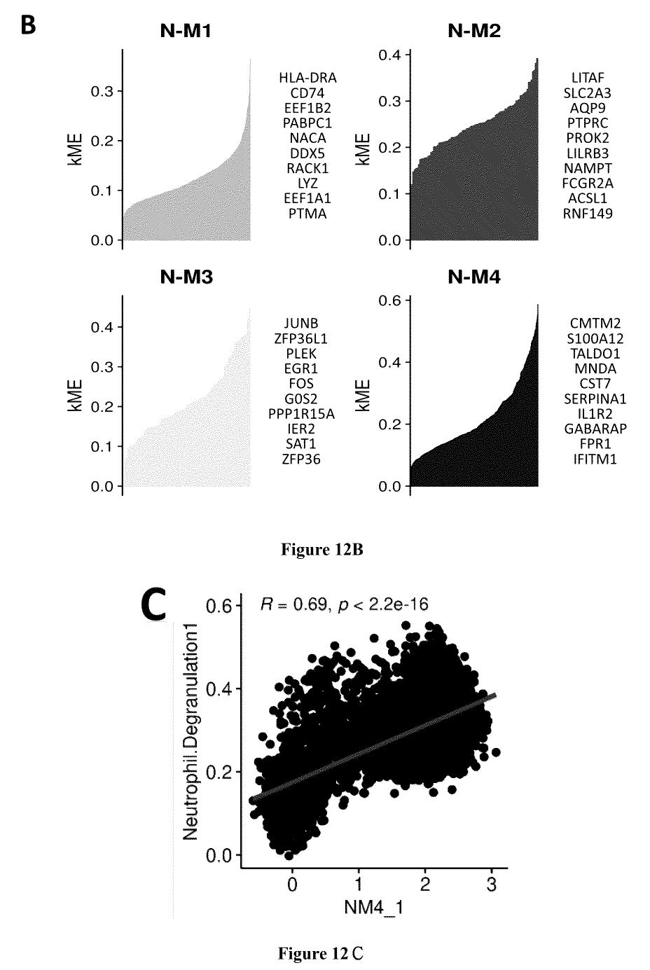

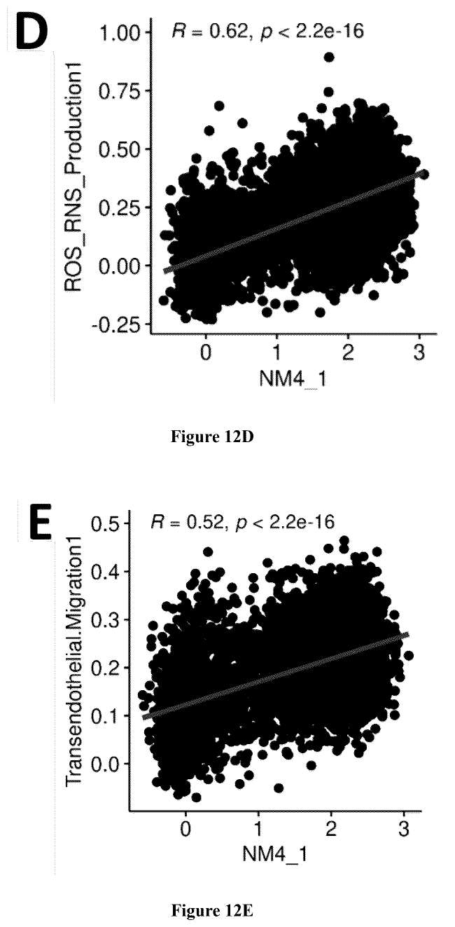

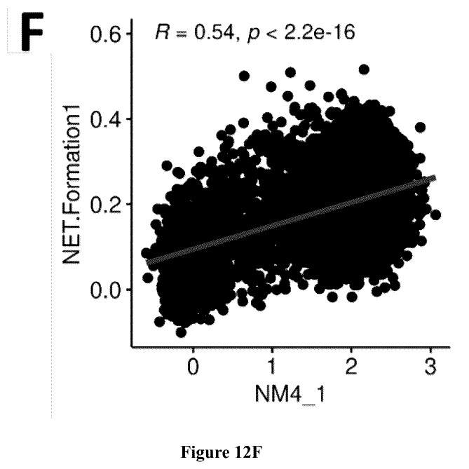

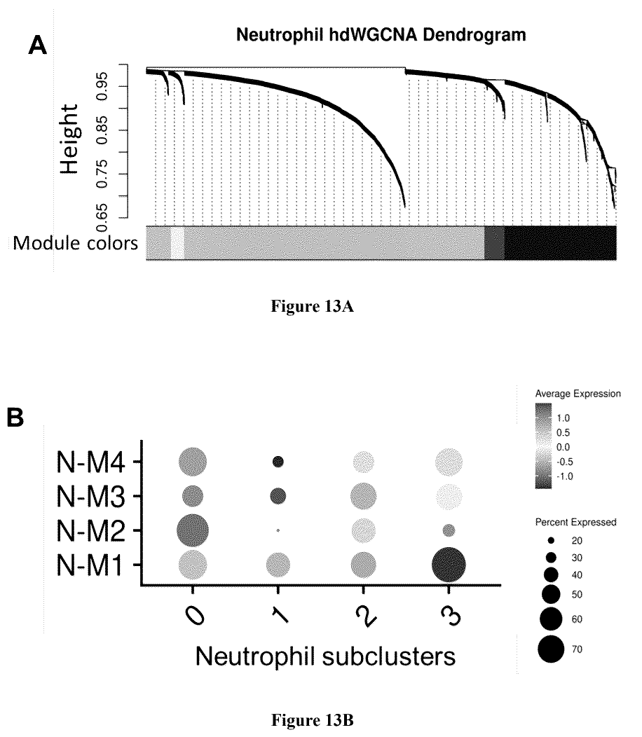

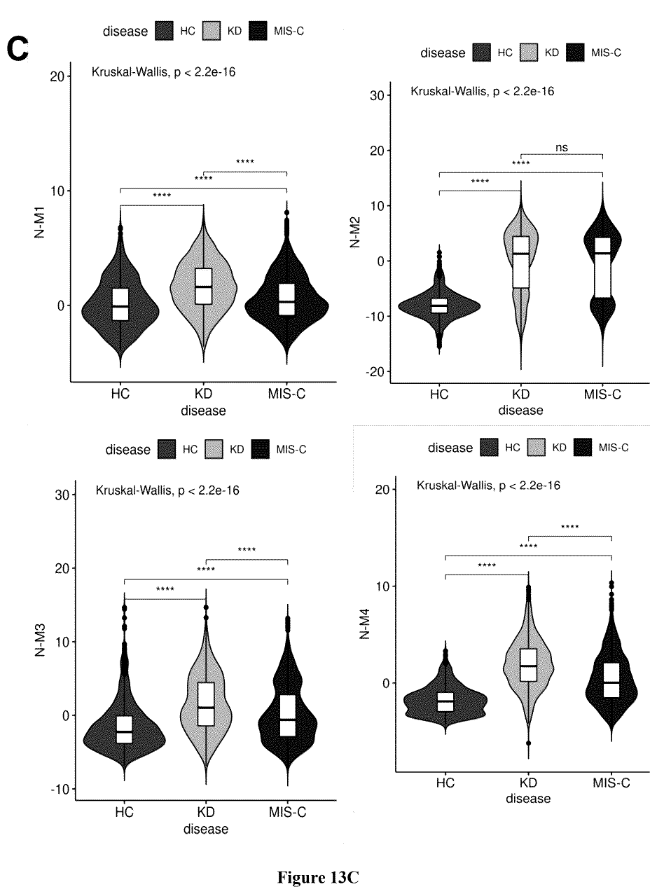

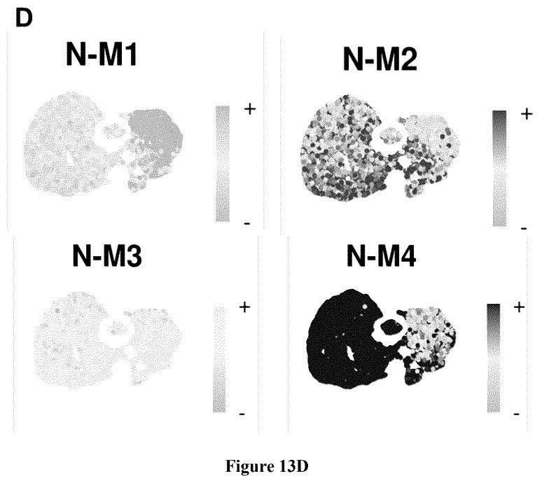

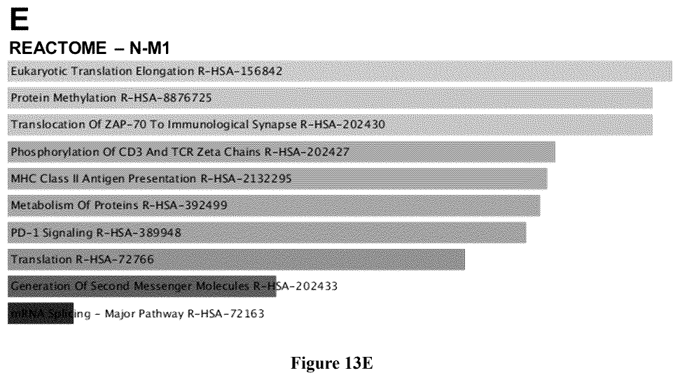

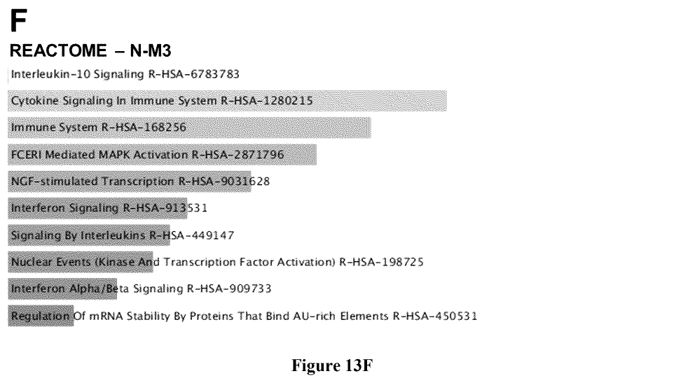

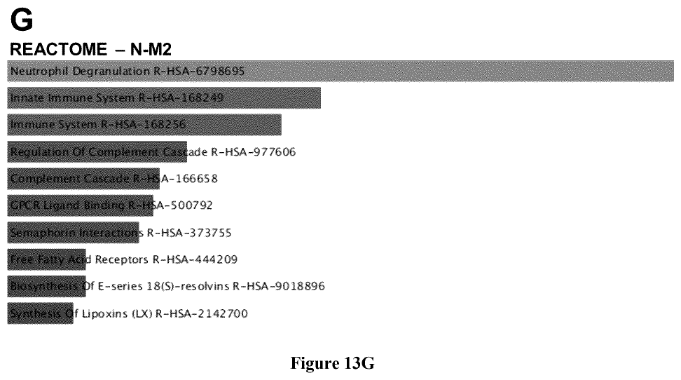

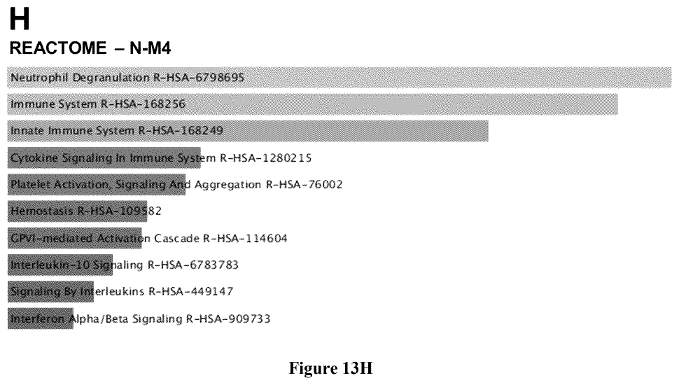

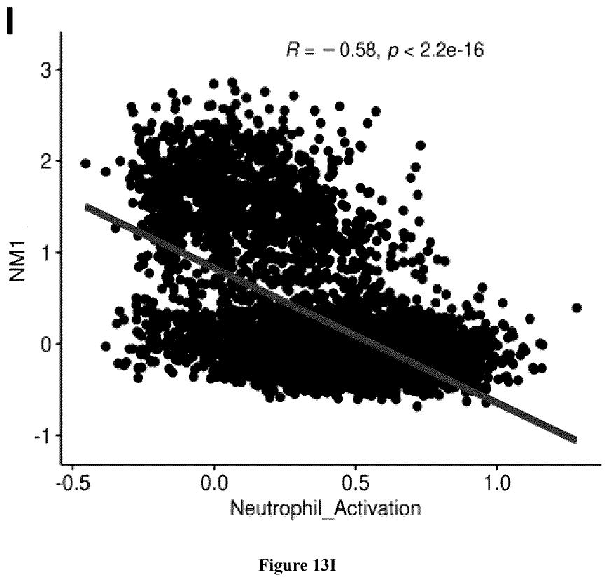

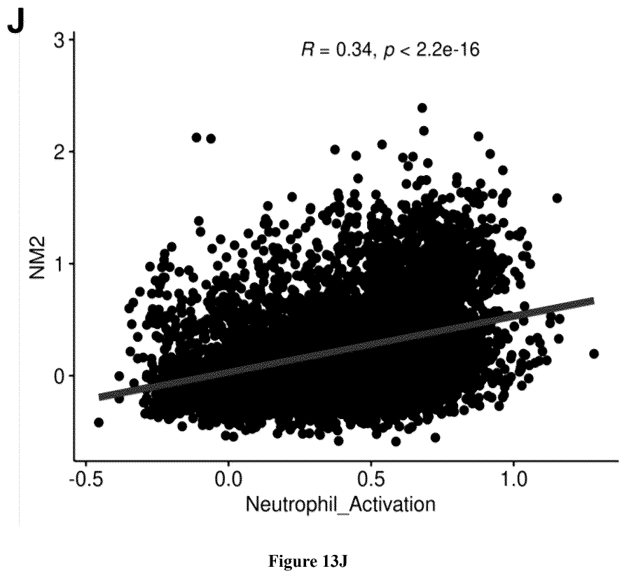

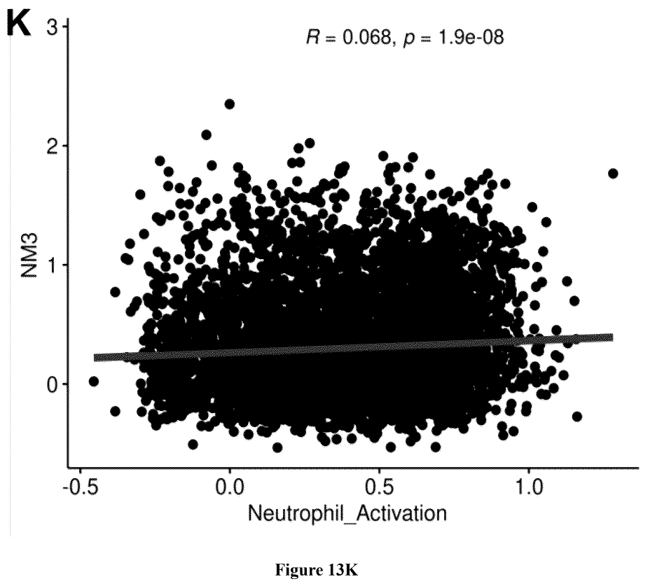

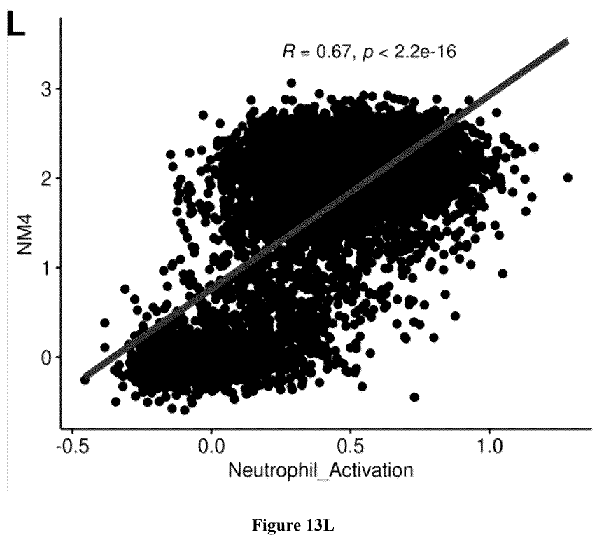

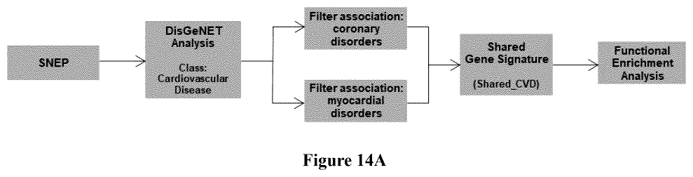

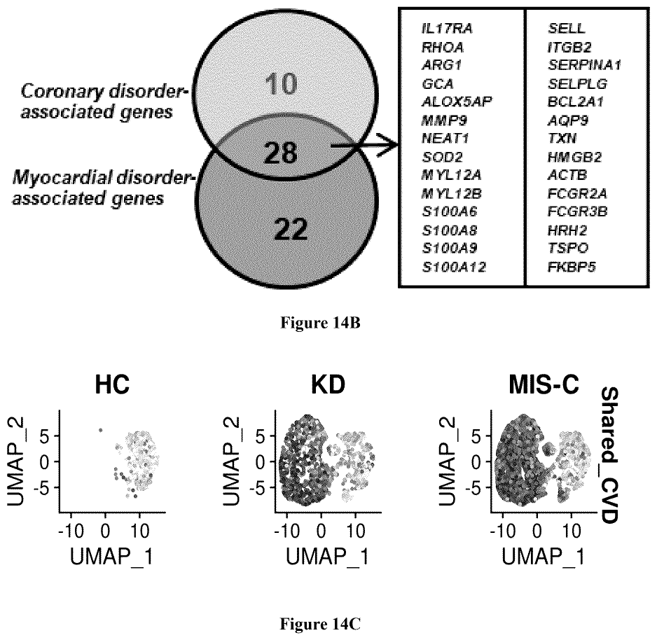

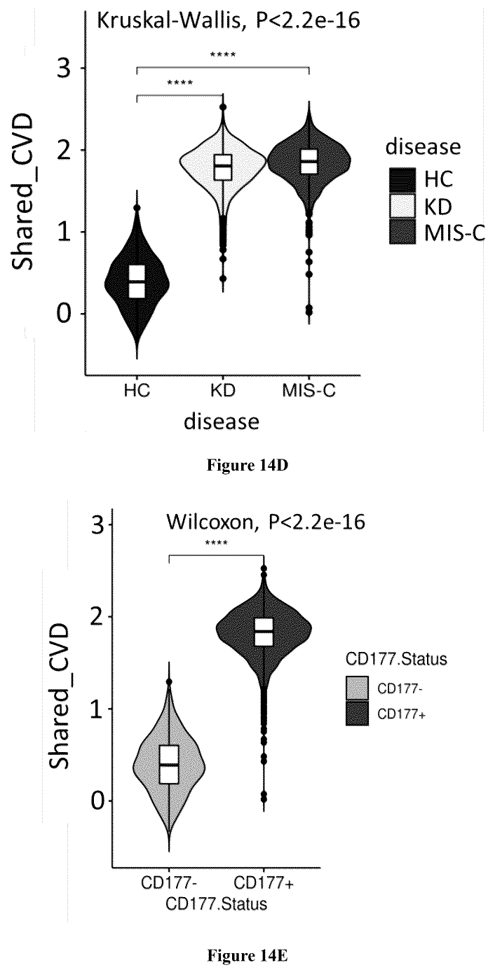

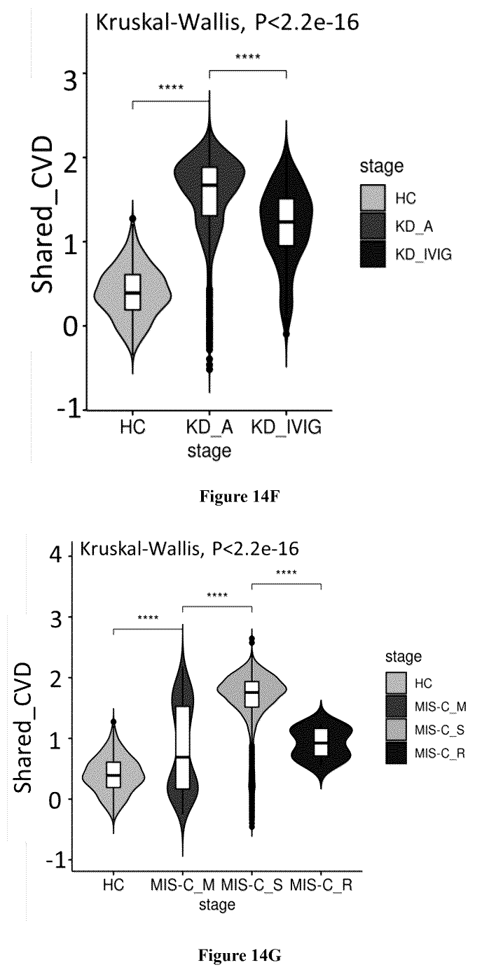

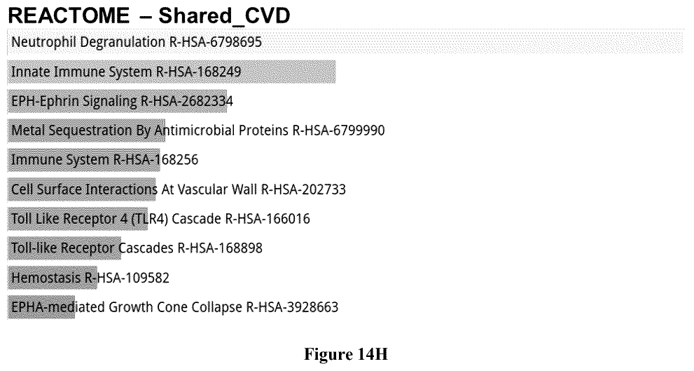

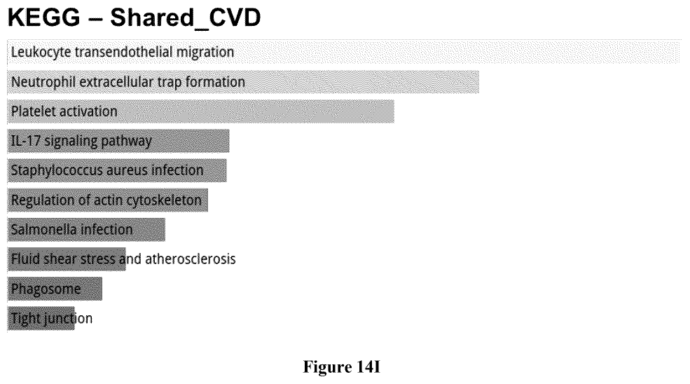

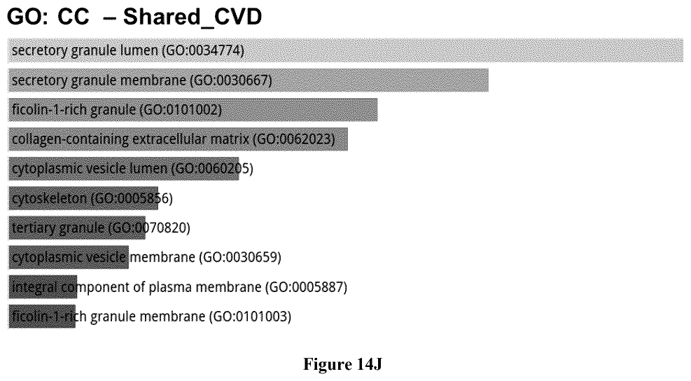

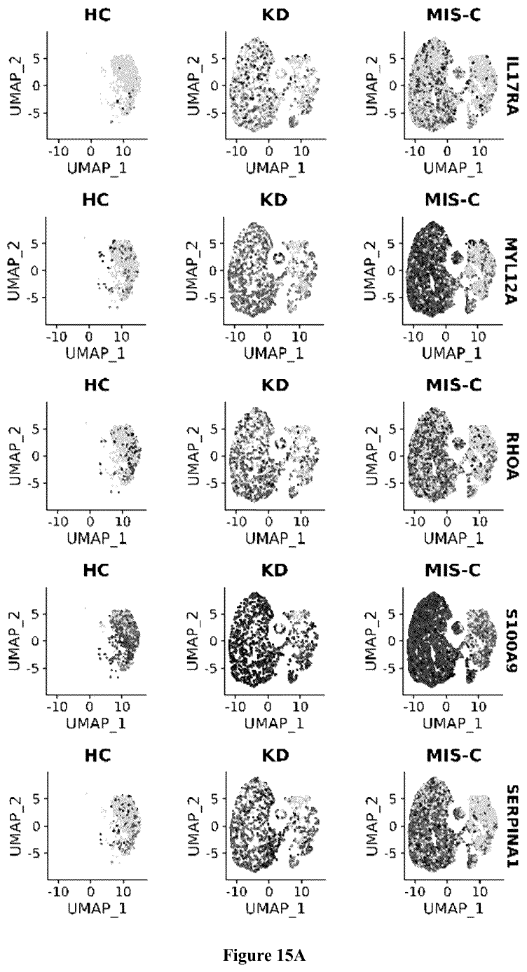

Network analysis of co-expressed genes may reveal critical genes, coactivated dependencies, or disease modules that may be required together to initiate a function or process.41 It may also reveal new functions or involvement of novel genes on a certain biological mechanism being studied. To identify the key genes that are crucial in neutrophil activation, we conducted a high dimensional weighted gene coexpression network analysis. After constructing the coexpression networks using soft power threshold=8 (FIG. 12A), results revealed 4 coexpression modules N-M1, N-M2, N-M3, and N-M4 (turquoise, maroon, yellow, and blue, respectively; FIG. 13A˜13B; Table 16). Each module is represented by the top 25 hub genes with the highest interconnectivity as ranked by eigengene-based connectivities (kME) (FIG. 12B). All modules have significant differences when comparing HC versus KD or MIS-C(FIG. 13C), wherein N-M1 is highly expressed in cluster 1 (FIG. 13D) which represents the neutrophils in HC group. N-M2 and N-M4 modules in both KD and MIS-C have the highest differences compared with HC (P<2.2e-16; FIGS. 13D and 13F) and may be more relevant to disease as supported by cluster expression (FIG. 13D) and functional enrichment (FIG. 13E-13H; Table 17). Pearson correlation analysis (FIG. 14I˜14L) revealed the highest positive correlation (R=0.67; P<2.2e-16) of N-M4 and neutrophil activation (FIG. 13L) and effector pathways (FIG. 12C˜12F). In addition, compared with N-M1, N-M2, and N-M3, all genes of N-M4 are included in SNEP (Table 16) and N-M4 expression correlates with KD and MIS-C prognosis.

TABLE 16
Hub genes in the four neutrophil modules
(N-M1, N-M2, N-M3, and N-M4)

| N-M1 (turquoise) | N-M2 (maroon) | N-M3 (yellow) | N-M4 (blue) |
|---|---|---|---|
| FCN1 | ALOX5 | ATP6V1B2 | ARHGDIB |
| RBM39 | CDC42EP3 | TAGAP | ITM2B |
| HLA-DRB1 | FFAR2 | ADGRE2 | CDA |
| ATP2B1 | TNFRSF10C | ICAM1 | GCA |
| PFDN5 | ATG16L2 | IVNS1ABP | GNG5 |
| TPT1 | TNFSF13B | CD82 | RHOG |
| VCAN | LIMK2 | PFKFB3 | NEAT1 |
| BTF3 | HK3 | IRF1 | TSPO |
| EIF1 | CREB5 | IL1RN | NCF1 |
| JUND | CXCR2 | CCR1 | SELL |
| EEF2 | CD55 | LCP2 | IFITM3 |
| CD52 | JAML | RGS2 | TXN |
| S100A10 | TUBA1A | KLF6 | MYL12A |
| HNRNPA1 | MTRNR2L12 | C5AR1 | ALOX5AP |
| YBX1 | CR1 | GLUL | FCER1G |
| HLA-DRA | LITAF | JUNB | CMTM2 |
| CD74 | SLC2A3 | ZFP36L1 | S100A12 |
| EEF1B2 | AQP9 | PLEK | TALDO1 |
| PABPC1 | PTPRC | EGR1 | MNDA |
| NACA | PROK2 | FOS | CST7 |
| DDX5 | LILRB3 | G0S2 | SERPINA1 |
| RACK1 | NAMPT | PPP1R15A | IL1R2 |
| LYZ | FCGR2A | IER2 | GABARAP |
| EEF1A1 | ACSL1 | SAT1 | FPR1 |
| PTMA | RNF149 | ZFP36 | IFITM1 |

Note:
Bold—Hub genes that overlap with SNEP
Underlined—Hub genes in SNEP that have FDA-approved drug(s)

TABLE 17
N-M4 module top 10 REACTOME pathways

| | | | Adj. | Odds | Combined | |
|---|---|---|---|---|---|---|
| Term | Overlap | P-value | P- value | Ratio | Score | Genes |
| Neutrophil | 9/468 | 2.85E−9 | 4.97E−7 | 23.92 | 470.55 | CDA; SERPINA1; FCER1G; |
| Degranulation | | | | | | GCA; SELL; FPR1; RHOG; |
| R-HSA-6798695 | | | | | | S100A12; MNDA |
| Immune | 14/1943 | 1.01E−8 | 8.80E−7 | 11.91 | 219.19 | IFITM3; CDA; IFITM1; |
| System | | | | | | SERPINA1; FCER1G; NCF1; |
| R-HSA-168256 | | | | | | GCA; IL1R2; FPR1; RHOG; |
| | | | | | | TALDO1; SELL; S100A12; |
| | | | | | | MNDA |
| Innate | 10/1035 | 2.12E−7 | 1.23E−5 | 12.33 | 189.43 | CDA; SERPINA1; FCER1G; |
| Immune | | | | | | NCF1; GCA; SELL; FPR1; |
| System | | | | | | RHOG; S100A12; MNDA |
| R-HSA-168249 | | | | | | |
| Cytokine | 6/702 | 1.83E−4 | 0.00796 | 8.75 | 75.28 | IFITM3; IFITM1; IL1R2; |
| Signaling In | | | | | | FPR1; S100A12; TALDO1 |
| Immune | | | | | | |
| System | | | | | | |
| R-HSA-1280215 | | | | | | |
| Platelet | 4/254 | 2.60E−4 | 0.00907 | 15.03 | 124.03 | SERPINA1; FCER1G; |
| Activation, | | | | | | GNG5; RHOG |
| Signaling And | | | | | | |
| Aggregation | | | | | | |
| R-HSA-76002 | | | | | | |
| Hemostasis | 5/576 | 6.41E−4 | 0.0181 | 8.50 | 62.46 | SERPINA1; FCER1G; |
| R-HSA-109582 | | | | | | GNG5; SELL; RHOG |
| GPVI- | 2/32 | 7.27E−4 | 0.0181 | 57.81 | 417.77 | FCER1G; RHOG |
| mediated | | | | | | |
| Activation | | | | | | |
| Cascade | | | | | | |
| R-HSA-114604 | | | | | | |
| Interleukin-10 | 2/45 | 0.00144 | 0.0313 | 40.31 | 263.82 | IL1R2; FPR1 |
| Signaling | | | | | | |
| R-HSA-6783783 | | | | | | |
| Signaling By | 4/453 | 0.00225 | 0.0435 | 8.28 | 50.49 | IL1R2; FPR1; S100A12; |
| Interleukins | | | | | | TALDO1 |
| R-HSA-449147 | | | | | | |
| Interferon | 2/72 | 0.00363 | 0.0607 | 24.73 | 138.90 | IFITM3; IFITM1 |
| Alpha/Beta | | | | | | |
| Signaling | | | | | | |
| R-HSA-909733 | | | | | | |

### Example 7

### CD177⁺ Neutrophils of KD and MIS-C are Associated with Cardiovascular Morbidities

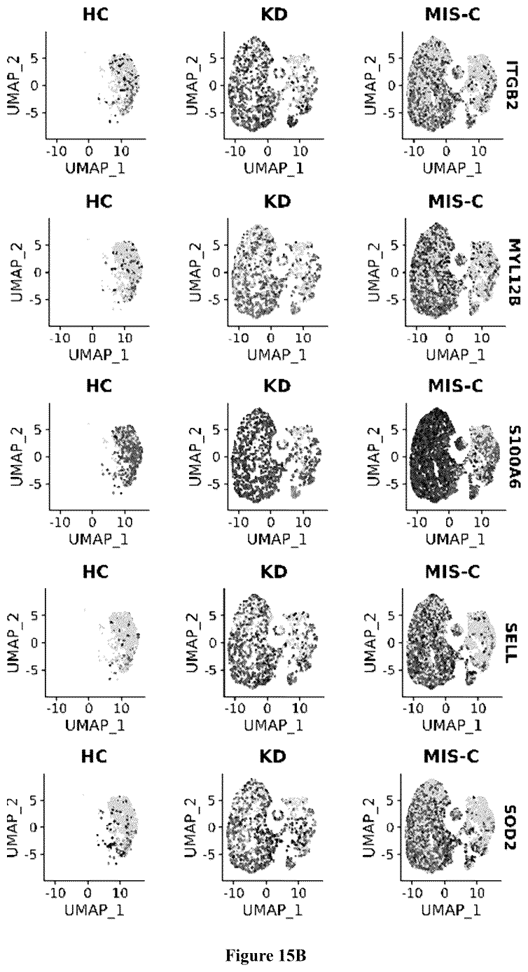

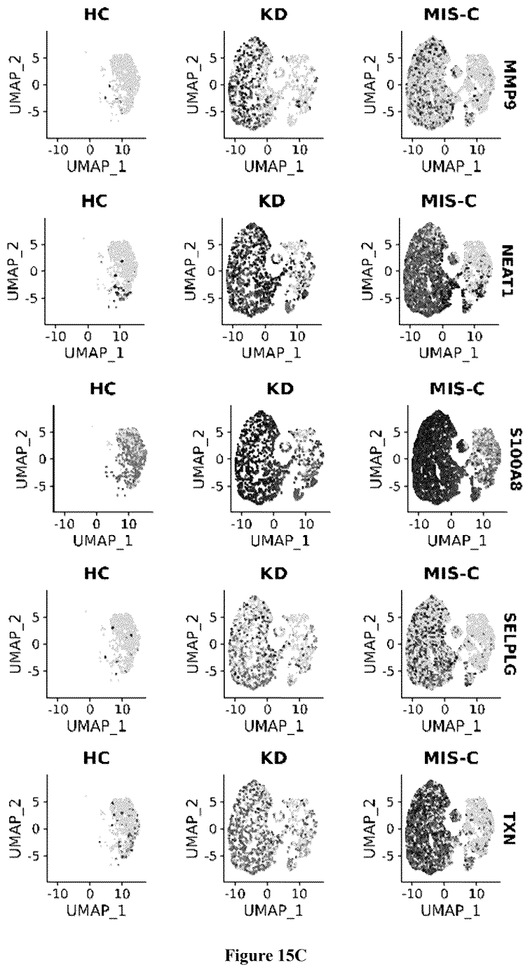

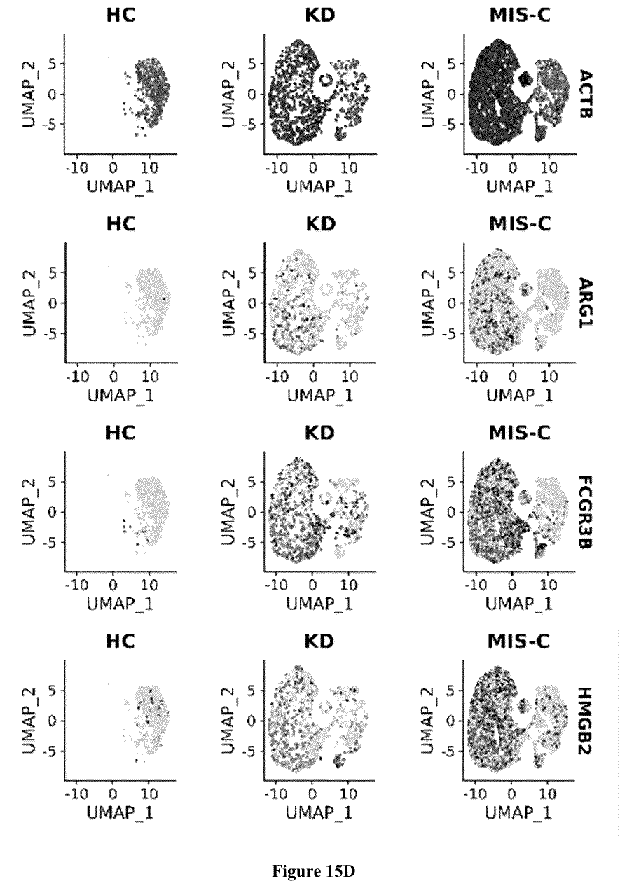

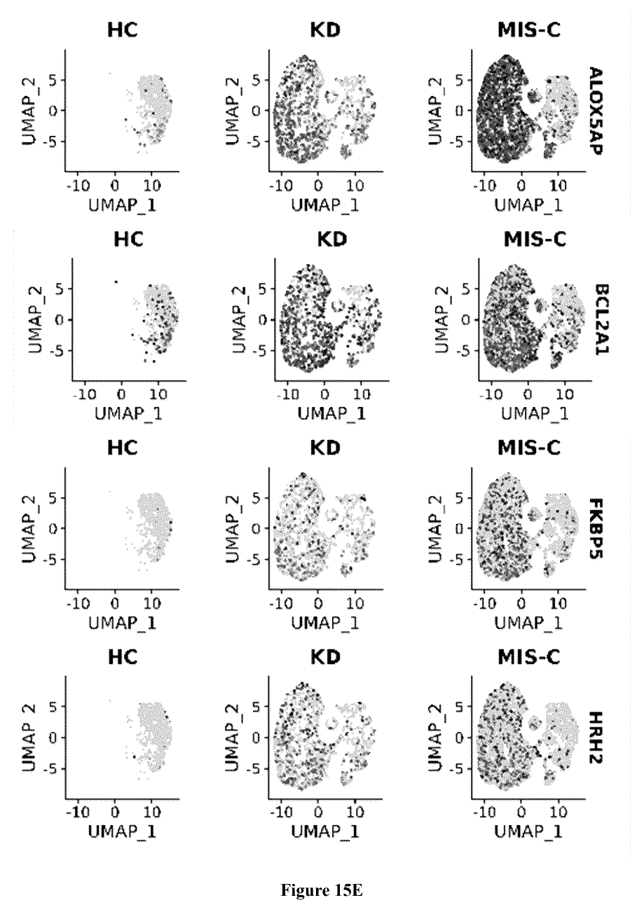

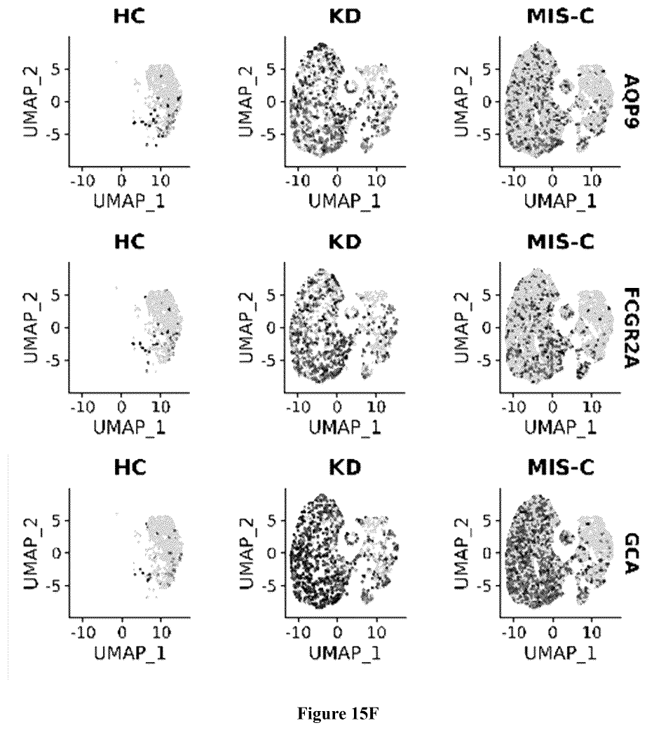

CAA and myocarditis are serious complications of KD and MIS-C. We investigated whether hyperactivated neutrophils and their induced transcriptional programs were associated with the observed cardiac pathologies in KD and MIS-C. We analyzed the cardiovascular disease association of SNEP with (1) coronary and (2) myocardial disorders using DisGeNET (FIG. 14A). We found the cardiac phenotypes CAA and myocarditis in the disease-gene interaction network. Of the 156 genes in the SNEP, 38 were associated with coronary disorders, 50 were mapped to myocardial issues, and 28 (Shared_CVD) were associated with both (FIG. 14B). The expression of the 28-gene signature was consistently upregulated in the CD177⁺ neutrophils of KD and MIS-C, but not in HC (P<2.2e-16; FIG. 14C˜1EG; FIG. 15A˜15F) and downregulated after KD IVIG treatment (P<2.2e-16; FIG. 14F). In addition, the expression of this signature increased with MIS-C severity and was ameliorated after recovery (P<2.2e-16; FIG. 14G).1.

To determine how the 28-gene signature can contribute to the development of coronary aneurysms and myocarditis, we performed a pathway enrichment analysis (FIG. 14H˜14J; Tables 18˜21). Consistent with our initial analysis, neutrophil degranulation (adjusted P<6.80e-8), leukocyte transendothelial migration (adjusted P<9.60e-7), and NET formation (adjusted P<9.80e-6) were highly activated by this signature (FIG. 14H˜14I). The EPH-ephrin (adjusted P<4.25e-4) and interleukin-17 (adjusted P<1.68e-4) signaling pathways were also highly activated. The data also indicated increased cell surface interactions at the vascular wall, which further supports our findings and mechanistic hypothesis regarding CD177⁺ neutrophil homing. Last, because degranulation is likely to drive disease pathogenesis, the specific granules used to trigger such phenotypes are crucial for understanding the mechanism. Neutrophils contain an array of granules with various functions. GO: Cellular Component analysis implicated the secretion of ficolin-1-rich granules (adjusted P<0.00247) and active release of tertiary granules (adjusted P<0.0135; FIG. 14J; Table 21).

TABLE 18
Top 10 REACTOME Pathways of the Shared_CVD (28-gene signature)

| | | | Adj. | Odds | Combined | |
|---|---|---|---|---|---|---|
| Term | Overlap | P-value | P- value | Ratio | Score | Genes |
| Neutrophil | 10/468 | 4.02E−10 | 6.80E−8 | 23.67 | 512.08 | SERPINA1; FCGR2A; |
| Degranulation | | | | | | GCA; SELL; ITGB2; |
| R-HSA-6798695 | | | | | | S100A12; MMP9; |
| | | | | | | S100A9; RHOA; |
| | | | | | | S100A8 |
| Innate | 10/1035 | 7.37E−7 | 6.22E−5 | 10.27 | 145.02 | SERPINA1; FCGR2A; |
| Immune | | | | | | GCA; SELL; ITGB2; |
| System | | | | | | S100A12; MMP9; |
| R-HSA-168249 | | | | | | S100A9; RHOA; |
| | | | | | | S100A8 |
| EPH-Ephrin | 4/91 | 7.55E−6 | 4.25E−4 | 38.09 | 449.27 | MMP9; MYL12A; |
| Signaling | | | | | | RHOA; MYL12B |
| R-HSA-2682334 | | | | | | |
| Metal | 2/6 | 2.83E−5 | 9.81E−4 | 384.0 | 4022.15 | S100A9; S100A8 |
| Sequestration | | | | | | |
| By | | | | | | |
| Antimicrobial | | | | | | |
| Proteins | | | | | | |
| R-HSA-6799990 | | | | | | |
| Immune | 11/1943 | 3.17E−5 | 9.81E−4 | 6.04 | 62.58 | SERPINA1; FCGR2A; |
| System | | | | | | GCA; SELL; ITGB2; |
| R-HSA-168256 | | | | | | S100A12; MMP9; |
| | | | | | | S100A9; RHOA; |
| | | | | | | S100A8; IL17RA |
| Cell Surface | 4/134 | 3.48E−5 | 9.81E−4 | 25.44 | 261.14 | SELPLG; SELL; |
| Interactions At | | | | | | ITGB2; S100A9 |
| Vascular Wall | | | | | | |
| R-HSA-202733 | | | | | | |
| Toll Like | 4/140 | 4.13E−5 | 9.98E−4 | 24.31 | 245.37 | ITGB2; S100A12; |
| Receptor 4 | | | | | | S100A9; S100A8 |
| (TLR4) | | | | | | |
| Cascade | | | | | | |
| R-HSA-166016 | | | | | | |
| Toll-like | 4/162 | 7.30E−5 | 0.00154 | 20.90 | 199.09 | ITGB2; S100A12; |
| Receptor | | | | | | S100A9; S100A8 |
| Cascades | | | | | | |
| R-HSA-168898 | | | | | | |
| Hemostasis | 6/576 | 1.2196382282832935E−4 | 0.002290209561998629 | 9.28 | 83.66 | SERPINA1; SELPLG; |
| R-HSA-109582 | | | | | | SELL; ITGB2; |
| | | | | | | S100A9; RHOA |
| EPHA- | 2/15 | 1.962E−4 | 0.00332 | 118.10 | 1008.13 | RHOA; MYL12B |
| mediated | | | | | | |
| Growth Cone | | | | | | |
| Collapse | | | | | | |
| R-HSA-3928663 | | | | | | |

TABLE 19
Top 10 KEGG Pathways of the Shared_CVD 28-gene signature

| | | | Adj. | Odds | Combined | |
|---|---|---|---|---|---|---|
| Term | Overlap | P-value | P- value | Ratio | Score | Genes |
| Leukocyte | 6/114 | 1.02E−8 | 9.60E−7 | 50.16 | 922.95 | ITGB2; MMP9; MYL12A; |
| transendothelial | | | | | | ACTB; RHOA; MYL12B |
| migration | | | | | | |
| Neutrophil | 6/189 | 2.08E−7 | 9.80E−6 | 29.49 | 453.69 | SELPLG; FCGR3B; |
| extracellular | | | | | | FCGR2A; ITGB2; AQP9; |
| trap formation | | | | | | ACTB |
| Platelet | 5/124 | 7.41E−7 | 2.32E−5 | 36.27 | 511.95 | FCGR2A; MYL12A; ACTB; |
| activation | | | | | | RHOA; MYL12B |
| IL-17 signaling | 4/94 | 8.59E−6 | 1.68E−4 | 36.82 | 429.48 | MMP9; S100A9; S100A8; |
| pathway | | | | | | IL17RA |
| Staphylococcus | 4/95 | 8.96E−6 | 1.68E−4 | 36.41 | 423.20 | SELPLG; FCGR3B; |
| aureus | | | | | | FCGR2A; ITGB2 |
| infection | | | | | | |
| Regulation | 5/218 | 1.18E−5 | 1.84E−4 | 20.17 | 228.88 | ITGB2; MYL12A; ACTB; |
| of actin | | | | | | RHOA; MYL12B |
| cytoskeleton | | | | | | |
| Salmonella | 5/249 | 2.23E−5 | 3.00E−4 | 17.58 | 188.23 | TXN; MYL12A; ACTB; |
| infection | | | | | | RHOA; MYL12B |
| Fluid shear | 4/139 | 4.02E−5 | 4.72E−4 | 24.49 | 247.89 | TXN; MMP9; ACTB; RHOA |
| stress and | | | | | | |
| atherosclerosis | | | | | | |
| Phagosome | 4/152 | 5.70E−5 | 5.95E−4 | 22.32 | 218.18 | FCGR3B; FCGR2A; ITGB2; |
| | | | | | | ACTB |
| Tight | 4/169 | 8.60E−5 | 8.08E−4 | 20.01 | 187.29 | MYL12A; ACTB; RHOA; |
| junction | | | | | | MYL12B |

TABLE 20
Top 10 GO: MF Pathways of the Shared_CVD 28-gene signature

| | | | Adj. | Odds | Combined | |
|---|---|---|---|---|---|---|
| Term | Overlap | P-value | P- value | Ratio | Score | Genes |
| RAGE | 4/9 | 3.85E−10 | 2.89E−8 | 665.57 | 14427.74 | HMGB2; S100A12; S100A9; |
| receptor | | | | | | S100A8 |
| binding | | | | | | |
| (GO: 0050786) | | | | | | |
| arachidonic | 3/6 | 4.90E−8 | 1.23E−6 | 798.76 | 13444.24 | ALOX5AP; S100A9; S100A8 |
| acid binding | | | | | | |
| (GO: 0050544) | | | | | | |
| icosatetraenoic | 3/6 | 4.90E−8 | 1.23E−6 | 798.76 | 13444.24 | ALOX5AP; S100A9; |
| acid binding | | | | | | S100A8 |
| (GO: 0050543) | | | | | | |
| icosanoid | 3/7 | 8.57E−8 | 1.61E−6 | 599.04 | 9748.00 | ALOX5AP; S100A9; |
| binding | | | | | | S100A8 |
| (GO: 0050542) | | | | | | |
| calcium ion | 6/348 | 7.25E−6 | 1.09E−4 | 15.65 | 185.26 | GCA; SELL; S100A6; |
| binding | | | | | | S100A12; S100A9; S100A8 |
| (GO: 0005509) | | | | | | |
| transition | 6/445 | 2.92E−5 | 3.65E−4 | 12.13 | 126.72 | ARG1; S100A12; SOD2; |
| metal ion | | | | | | MMP9; S100A9; S100A8 |
| binding | | | | | | |
| (GO: 0046914) | | | | | | |
| metal ion | 6/517 | 6.73E−5 | 7.21E−4 | 10.39 | 99.78 | GCA; SELL; S100A6; |
| binding | | | | | | S100A12; S100A9; S100A8 |
| (GO: 0046872) | | | | | | |
| Toll-like | 2/11 | 1.03E−4 | 9.67E−4 | 170.62 | 1566.22 | S100A9; S100A8 |
| receptor | | | | | | |
| binding | | | | | | |
| (GO: 0035325) | | | | | | |
| zinc ion | 4/336 | 0.00116 | 0.00971 | 9.86 | 66.60 | S100A12; MMP9; S100A9; |
| binding | | | | | | S100A8 |
| (GO: 0008270) | | | | | | |
| manganese ion | 2/48 | 0.00205 | 0.0154 | 33.32 | 206.27 | ARG1; SOD2 |
| binding | | | | | | |
| (GO: 0030145) | | | | | | |

TABLE 21
Top 10 GO: CC Pathways of the Shared_CVD 28-gene signature

| | | | Adj. | Odds | Combined | |
|---|---|---|---|---|---|---|
| Term | Overlap | P-value | P- value | Ratio | Score | Genes |
| secretory | 6/316 | 4.169E−6 | 2.58E−4 | 17.30 | 214.28 | SERPINA1; GCA; ARG1; |
| granule lumen | | | | | | S100A12; S100A9; S100A8 |
| (GO: 0034774) | | | | | | |
| secretory | 5/274 | 3.532E−5 | 0.00110 | 15.92 | 163.23 | FCGR3B; FCGR2A; SELL; |
| granule | | | | | | ITGB2; RHOA |
| membrane | | | | | | |
| (GO: 0030667) | | | | | | |
| ficolin-1-rich | 4/184 | 1.194E−4 | 0.00247 | 18.33 | 165.53 | SERPINA1; ITGB2; MMP9; |
| granule | | | | | | RHOA |
| (GO: 0101002) | | | | | | |
| collagen- | 5/380 | 1.653E−4 | 0.00256 | 11.36 | 98.92 | SERPINA1; S100A6; |
| containing | | | | | | MMP9; S100A9; S100A8 |
| extracellular | | | | | | |
| matrix | | | | | | |
| (GO: 0062023) | | | | | | |
| cytoplasmic | 3/115 | 5.463E−4 | 0.00677 | 21.28 | 159.85 | S100A12; S100A9; S100A8 |
| vesicle lumen | | | | | | |
| (GO: 0060205) | | | | | | |
| cytoskeleton | 5/600 | 0.00132 | 0.0135 | 7.08 | 46.91 | S100A12; S100A9; ACTB; |
| (GO: 0005856) | | | | | | RHOA; S100A8 |
| tertiary | 3/164 | 0.00153 | 0.0135 | 14.77 | 95.76 | ITGB2; MMP9; RHOA |
| granule | | | | | | |
| (GO: 0070820) | | | | | | |
| cytoplasmic | 4/380 | 0.00183 | 0.0142 | 8.69 | 54.75 | FCGR3B; FCGR2A; SELL; |
| vesicle | | | | | | RHOA |
| membrane | | | | | | |
| (GO: 0030659) | | | | | | |
| integral | 7/1454 | 0.00322 | 0.0200 | 4.27 | 24.48 | SELPLG; FCGR3B; |
| component of | | | | | | FCGR2A; SELL; HRH2; |
| plasma | | | | | | AQP9; IL17RA |
| membrane | | | | | | |
| (GO: 0005887) | | | | | | |
| ficolin-1-rich | 2/61 | 0.00329 | 0.0200 | 25.97 | 148.45 | ITGB2; RHOA |
| granule | | | | | | |
| membrane | | | | | | |
| (GO: 0101003) | | | | | | |

### Example 8

### Identification of Candidate FDA-Approved Drugs for the Treatment of KD and MIS-C

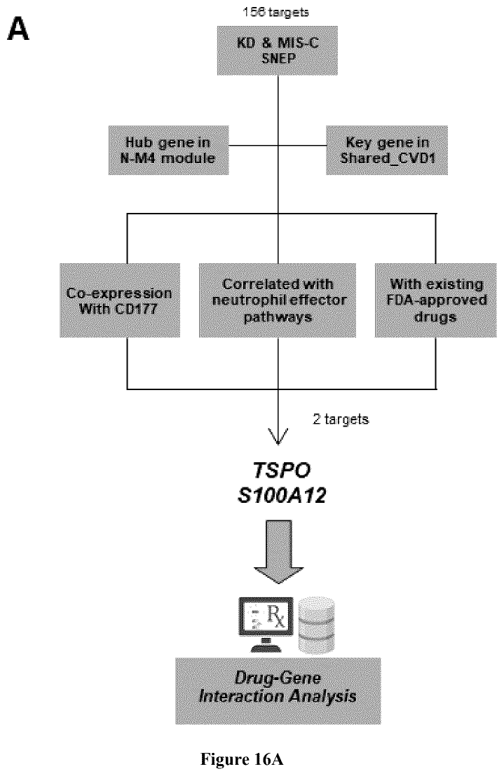

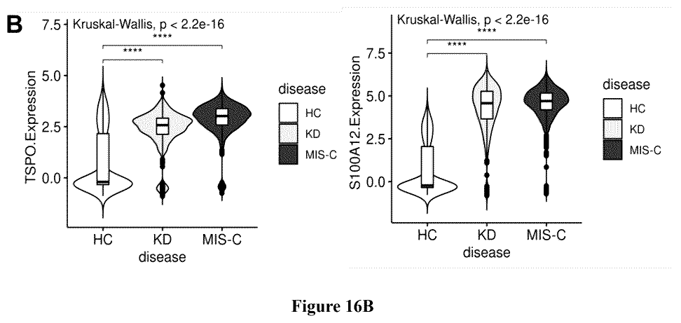

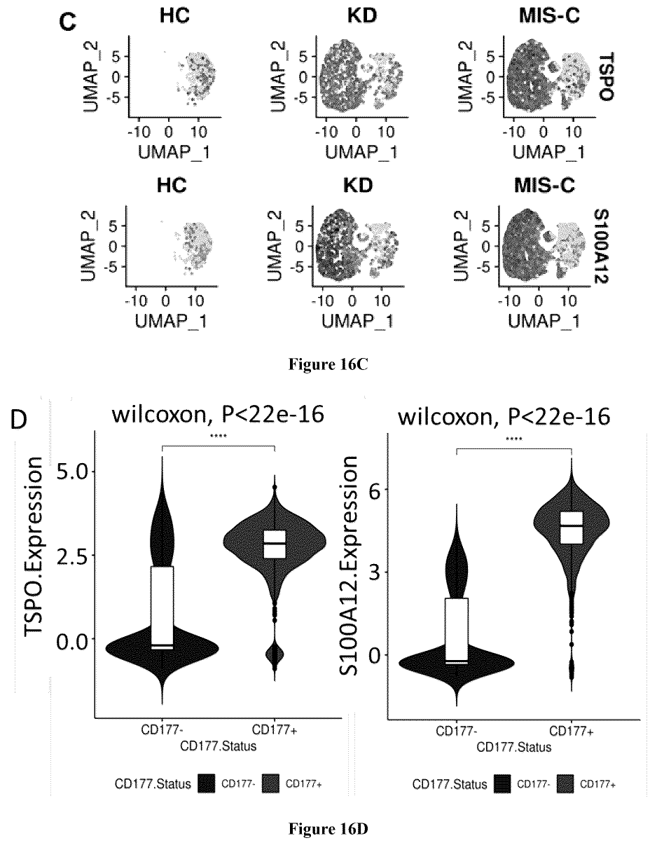

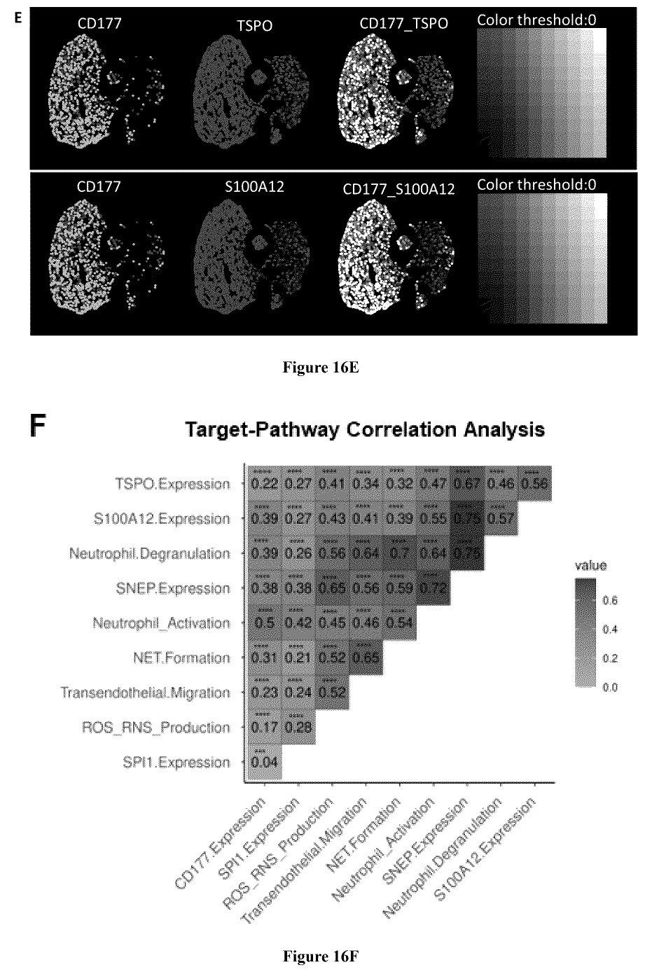

On the basis of the preceding findings, CD177⁺ neutrophils are potentially an important driver in the pathogenesis of KD and MIS-C through their enhanced and hyperactive effector state. Using the SNEP, we identified FDA-approved drugs that could be repurposed as neutrophil-targeted therapies to ameliorate neutrophil hyperactivation in both diseases (FIG. 16). We assessed the potential druggability of the genes shared in KD and MIS-C neutrophils using 19 categories in the DGIdb. Of the 156 genes, 45 were classified as druggable genome, 22 as enzyme, 10 as kinase, 9 as clinically actionable, 8 as cell surface, 6 as external side of plasma membrane, 5 as protease, 4 as protease inhibitor, 4 as transporter, 3 as drug resistance, 3 as G protein-coupled receptor, 2 as tumor suppressor, 1 as growth factor, 1 as ion channel, 1 as neutral zinc metallopeptidase, 1 as thioredoxin, 4 as transcription factor, and 5 as transcription factor complex. We performed a target and drug screening using specific criteria (FIG. 16A). Of 156 genes, 34 have at least one FDA-approved drug. A total of 312 FDA-approved drugs could potentially interact with the 34 targets. Across all 312 drugs, 17 FDA-approved drugs have >1 target: methotrexate, doxorubicin, gemcitabine, abacavir, adalimumab, alprazolam, clotrimazole, clozapine, disulfiram, etanercept, ibuprofen, idarubicin, infliximab, methazolamide, penicillin G potassium, streptonigrin, and sulfinpyrazone, wherein methotrexate has the greatest number of interacting gene partners.

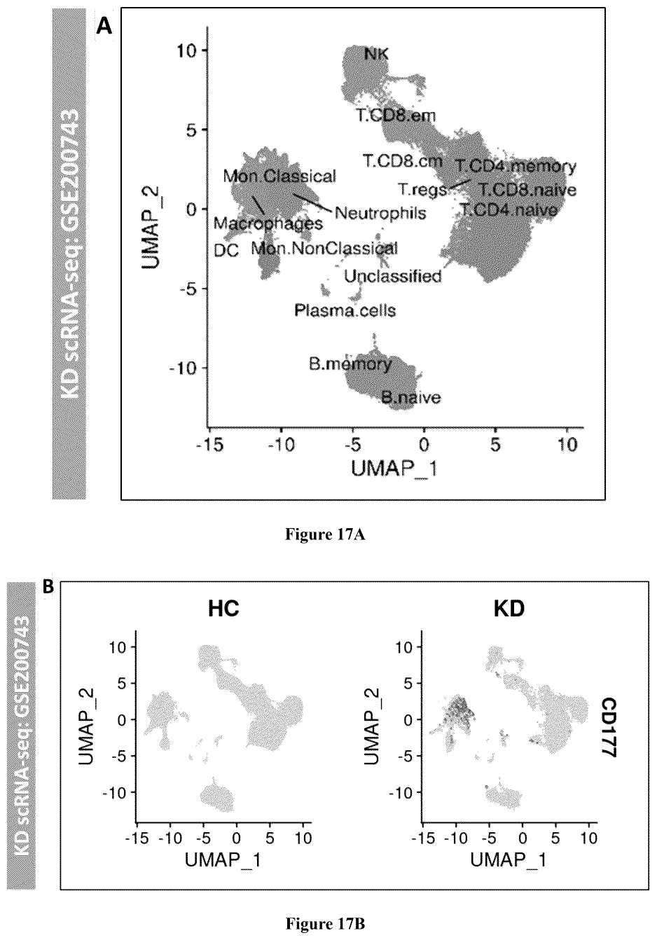

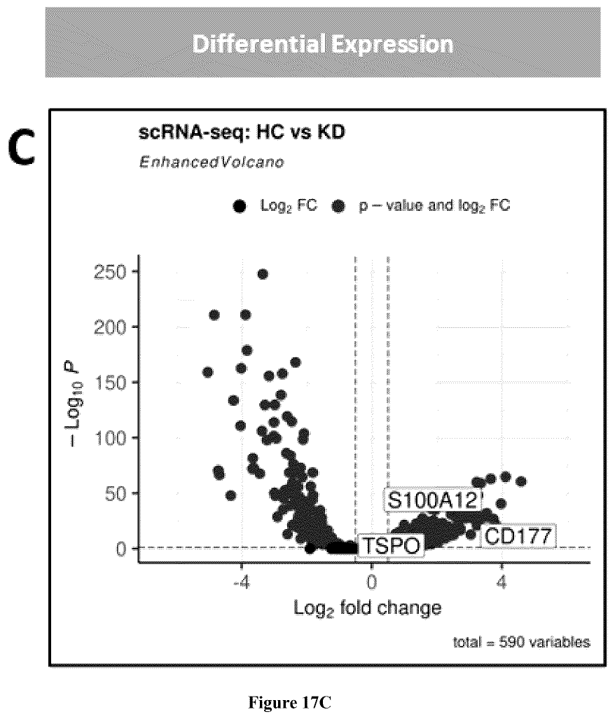

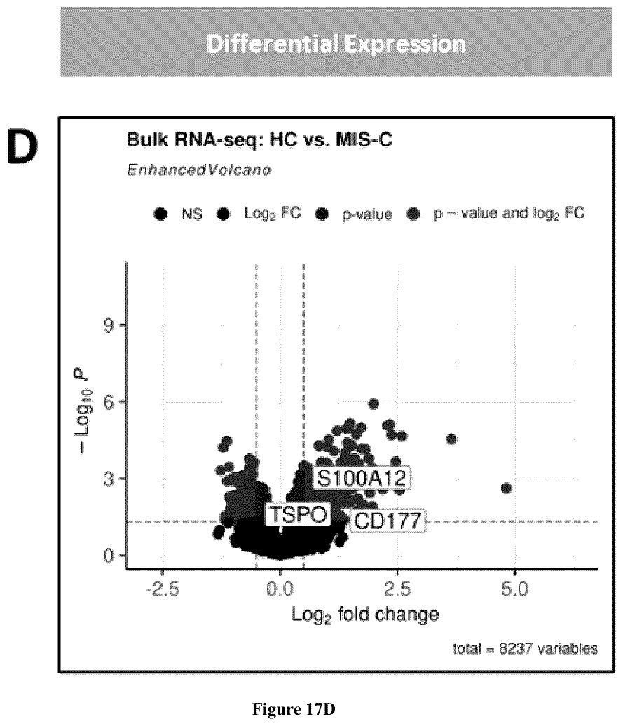

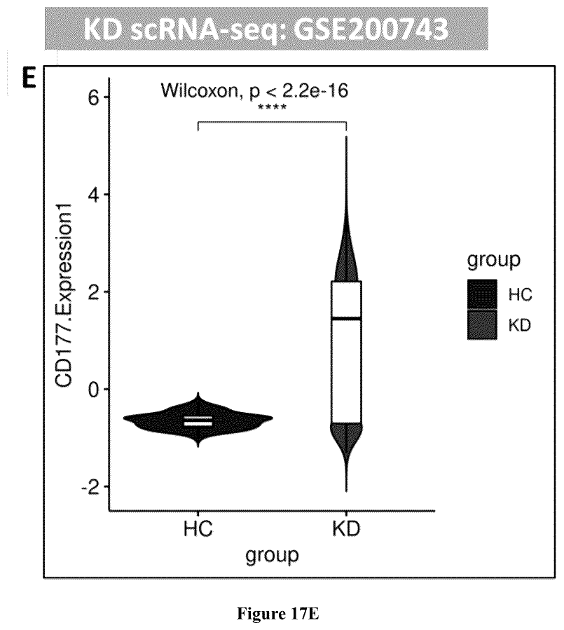

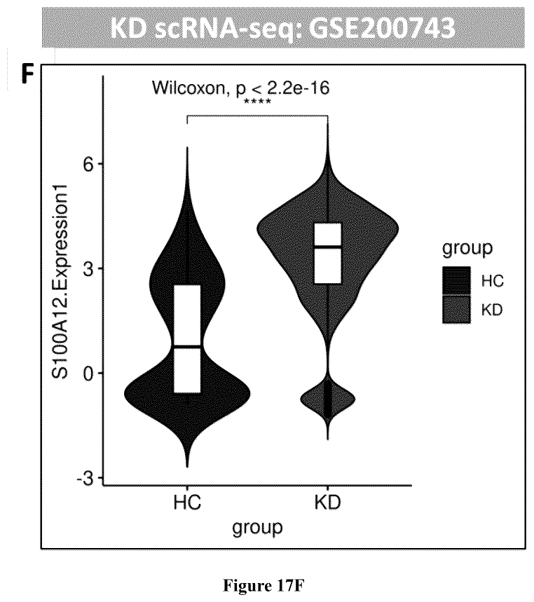

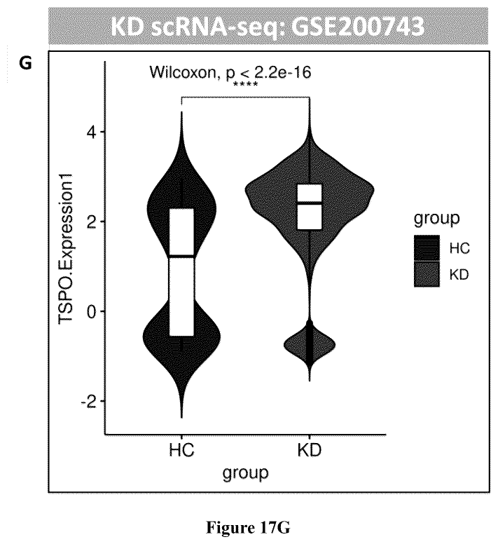

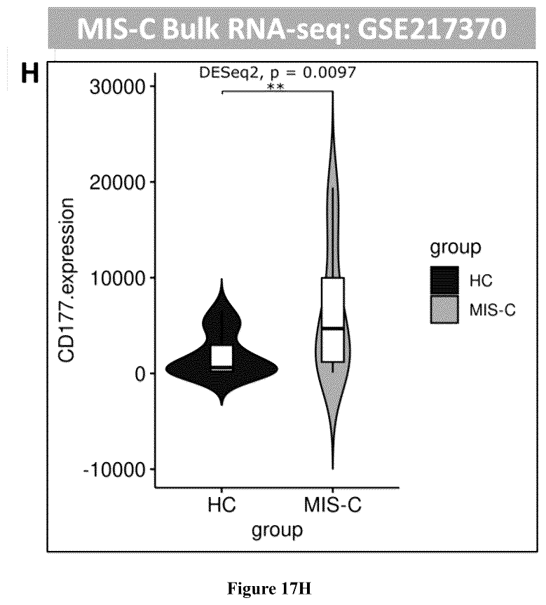

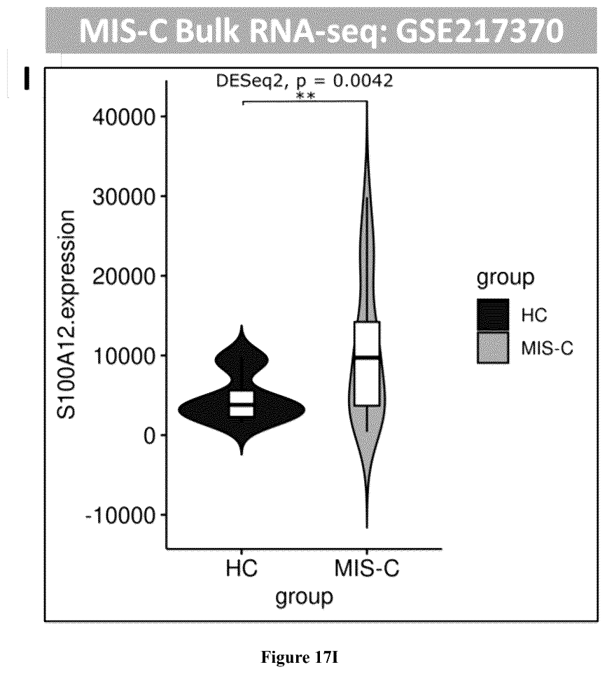

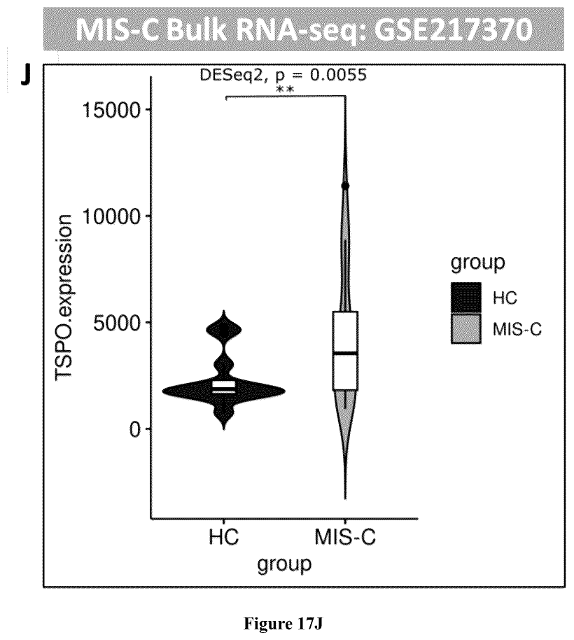

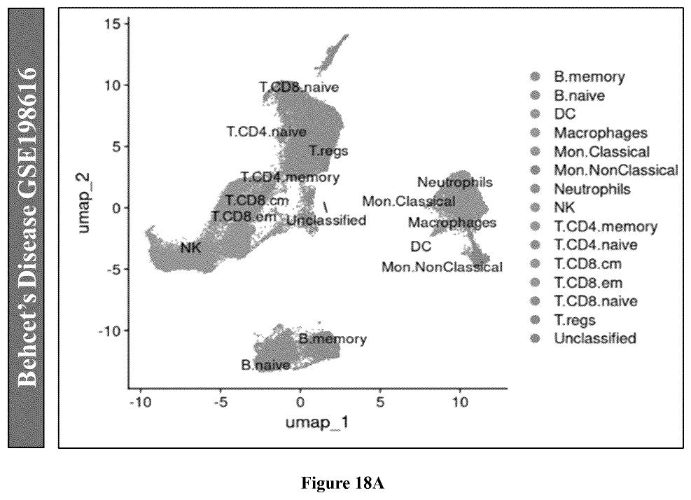

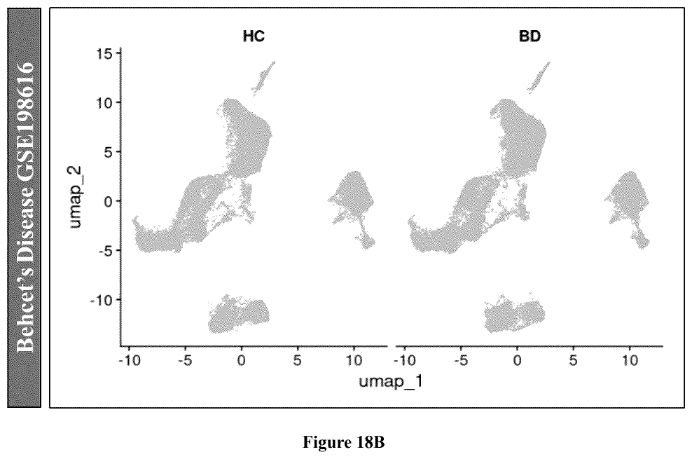

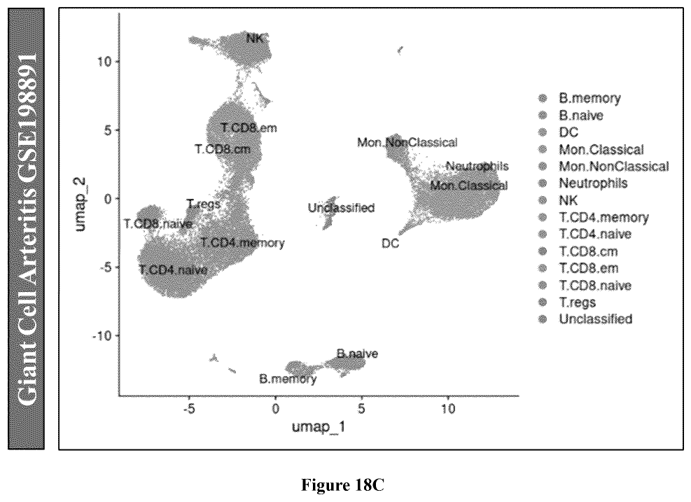

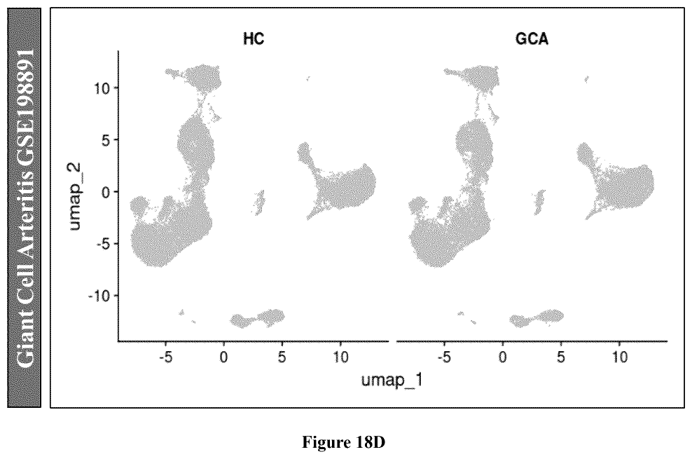

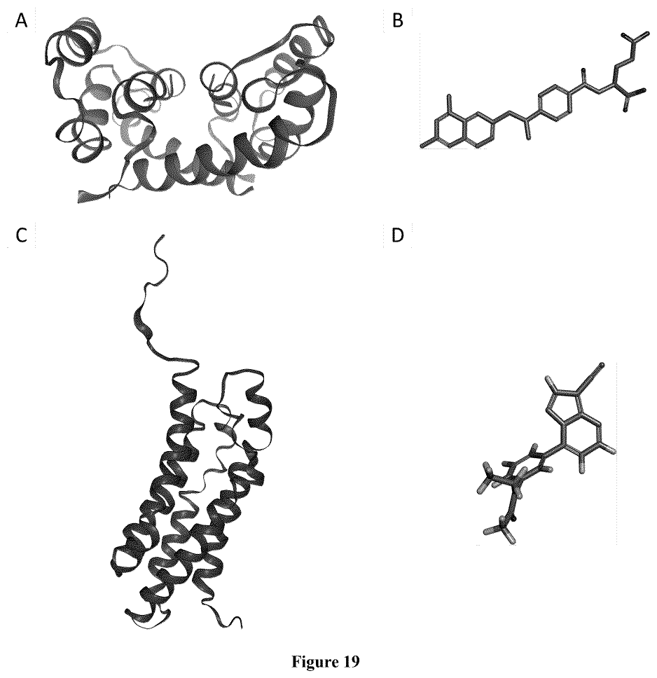

We then narrowed down the drug targets by selection criteria and finalized with 2 targets: TSPO (translocator protein) and S100A12 (S100 calcium-binding protein A12; FIG. 16A). KD and MIS-C have highly significant upregulated TSPO and S100A12 expression levels compared with control children (P<2.2e-16; FIG. 16B) and their expression is highest in CD177⁺ neutrophils (FIGS. 16C and 16D). We also demonstrated that TSPO and S100A12 are both coexpressed with CD177 (FIG. 16E). Furthermore, we reanalyzed 2 independent cohorts that were obtained from NCBI public database (KD scRNA-seq data with GEO ID “GSE200743” and the MIS-C Bulk RNA-seq with GEO ID “GSE217370”) and validated the existence of CD177⁺ neutrophils, as well as the targets TSPO and S100A12 (FIG. 17). Meanwhile, we also found that CD177⁺ neutrophils are more specific to KD and MIS-C than adult-onset systemic vasculitis, such as Behcet disease and giant cell arteritis (FIG. 18).

We then performed Pearson correlation analysis to identify the significance of targeting TSPO and S100A12 against neutrophil hyperactivation (FIG. 16F). The drug targets are positively correlated with the upregulation of neutrophil effector functions, including neutrophil degranulation (R=0.46, R=0.57, and P<0.0001, respectively), transendothelial migration (R=0.34, R=0.41, and P<0.0001, respectively), ROS/RNS production (R=0.41, R=0.43, and P<0.0001, respectively), and NET formation (R=0.32, R=39, and P<0.0001, respectively), CD177 expression (R=0.22, R=0.39, and P<0.0001, respectively), SPI1 expression (R=0.27, R=0.26, and P<0.0001, respectively), SNEP expression (R=0.67, R=0.75, and P<0.0001, respectively), and neutrophil activation (R-0.47, R=0.55, and P<0.0001, respectively), which further support its potential to control the aberrant neutrophil functions during KD and MIS-C(FIG. 16F).

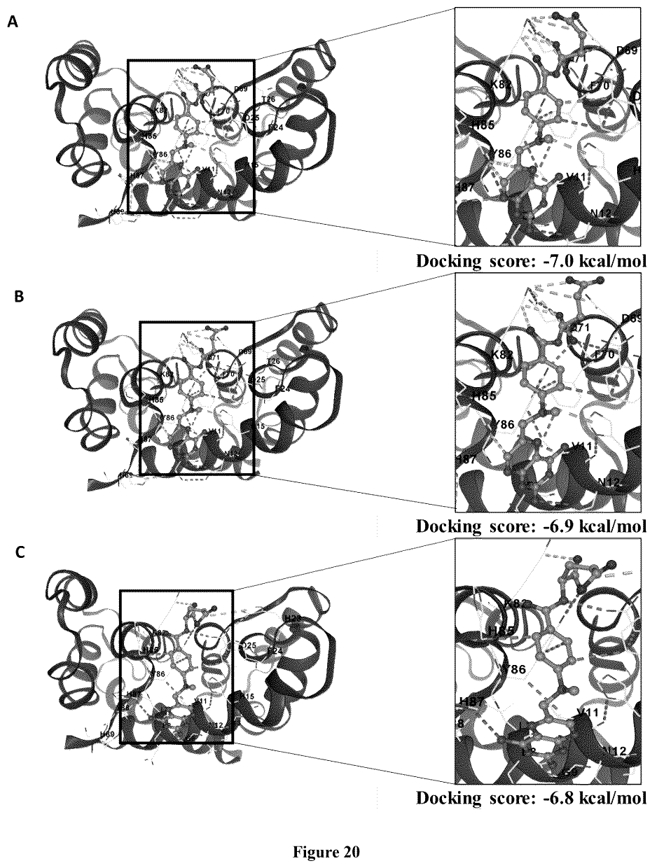

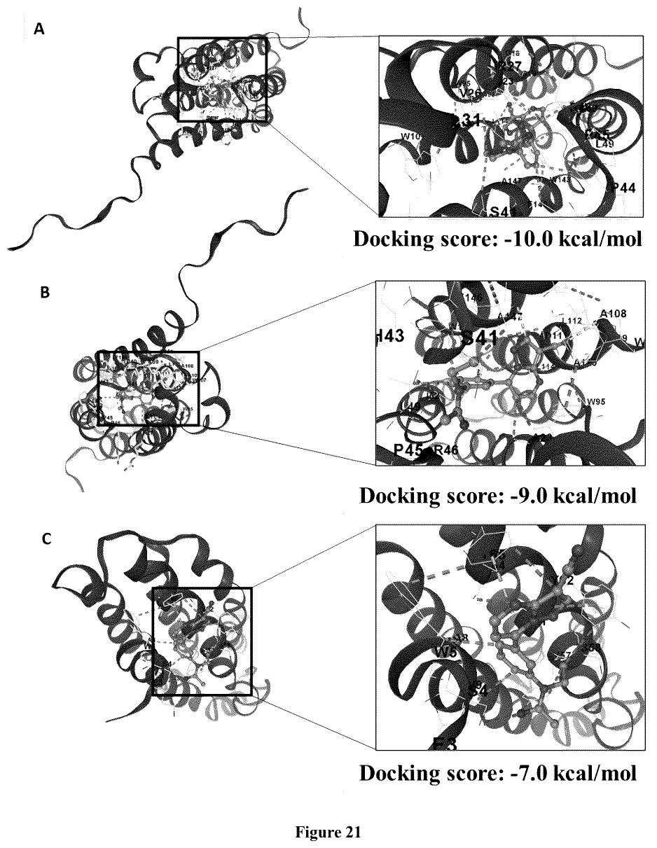

After filtering, the FDA-approved drugs are further narrowed down to methotrexate for S100A12 and 18 drugs (zolpidem, estazolam, flumazenil, midazolam, lorazepam, clotiazepam, zaleplon, halazepam, alprazolam, metronidazole, oxazepam, quazepam, flurazepam, prazepam, clonazepam, disulfiram, temazepam, and chlordiazepoxide) for TSPO. To determine whether the suggested drugs could bind to the drug targets, we performed molecular docking of methotrexate and zaleplon to S100A12 and TSPO, respectively (FIG. 19). Results suggest potential strong binding affinities of methotrexate (top: −7 kcal/mol; FIG. 20; Table 22) and zaleplon (top: −10 kcal/mol; FIG. 21; Table 23).

TABLE 22
Molecular docking summary results of S100A12 and methotrexate

| CurPocket | Vina | Cavity | Center | Docking size | |
|---|---|---|---|---|---|
| ID | score | volume (Å3) | (x, y, z) | (x, y, z) | Contact residues |
| C5 | −7.0 | 73 | 14, 11, 15 | 28, 28, 28 | Chain A: LYS82 HIS85 |
| | | | | | TYR86 HIS87 HIS89 |
| | | | | | GLU8 VAL11 ASN12 |
| | | | | | HIS15 PHE24 ASP25 |
| | | | | | THR26 ASP69 PHE70 |
| | | | | | GLN71 |
| C4 | −6.9 | 83 | 8, 22, 13 | 28, 28, 28 | Chain A: LYS82 HIS85 |
| | | | | | TYR86 HIS87 HIS89 |
| | | | | | GLU8 VAL11 ASN12 |
| | | | | | HIS15 PHE24 ASP25 |
| | | | | | THR26 ASP69 PHE70 |
| | | | | | GLN71 |
| C3 | −6.8 | 115 | 20, 28, −9 | 28, 28, 28 | Chain A: LEU7 GLU8 |
| | | | | | GLY9 VAL11 ASN12 |
| | | | | | ILE13 HIS15 HIS23 |
| | | | | | PHE24 ASP25 LYS82 |
| | | | | | HIS85 TYR86 HIS87 |
| | | | | | THR88 HIS89 |
| C1 | −6.5 | 223 | 7, 11, −1 | 28, 28, 28 | Chain A: LEU32 LEU36 |
| | | | | | GLU55 ILE56 GLN58 |
| | | | | | GLY59 LEU60 ALA62 |
| | | | | | GLU72 PHE73 ILE74 |
| | | | | | SER75 VAL77 ILE79 |
| C2 | −6.5 | 154 | 2, 29, 0 | 28, 28, 28 | Chain A: LEU32 LEU36 |
| | | | | | ALA51 VAL52 ILE53 |
| | | | | | GLU55 ILE56 GLY59 |
| | | | | | LEU60 GLU72 PHE73 |
| | | | | | ILE74 SER75 VAL77 |
| | | | | | ALA78 ILE79 ALA80 |
| | | | | | LYS82 ALA83 |

TABLE 23
Molecular docking summary results of TSPO and zaleplon

| CurPocket | Vina | Cavity | Center | Docking size | |
|---|---|---|---|---|---|
| ID | score | volume (Å3) | (x, y, z) | (x, y, z) | Contact residues |
| C1 | −10.0 | 1173 | 8, 2, 7 | 22, 22, 22 | Chain A: GLY18 GLY19 |
| | | | | | ALA23 VAL26 ARG27 |
| | | | | | LEU31 LYS39 SER41 |
| | | | | | PRO44 PRO45 ARG46 |
| | | | | | LEU49 ALA50 ILE52 |
| | | | | | TRP53 TRP93 TRP95 |
| | | | | | TRP107 LEU114 |
| | | | | | TRP143 PHE146 |
| | | | | | ALA147 |
| | | | | | LEU150 |
| C2 | −9.0 | 190 | 12, 13, 0 | 22, 22, 22 | Chain A: ALA23 VAL26 |
| | | | | | LEU31 PRO40 SER41 |
| | | | | | HIS43 PRO44 PRO45 |
| | | | | | ARG46 LEU49 TRP95 |
| | | | | | TRP107 ALA108 |
| | | | | | LEU109 ALA110 |
| | | | | | ASP111 TRP143 |
| | | | | | PHE146 |
| | | | | | ALA147 LEU150 |
| | | | | | ASN151 |
| C4 | −6.1 | 175 | 1, 12, −5 | 22, 22, 22 | Chain A: GLY106 |
| | | | | | ALA108 LEU109 |
| | | | | | ASP111 LEU112 |
| | | | | | THR148 VAL149 |
| | | | | | ASN151 |
| | | | | | TYR152 TRP155 |
| C5 | −7.0 | 147 | −5, −18, 7 | 22, 22, 22 | Chain A: GLU3 SER4 |
| | | | | | TRP5 ALA8 VAL9 |
| | | | | | TYR57 SER58 GLY61 |
| | | | | | TYR62 SER64 |
| | | | | | TYR65 |
| C3 | −6.2 | 137 | −6, −3, 8 | 22, 22, 22 | Chain A: PRO44 THR48 |
| | | | | | LEU49 ILE52 THR55 |
| | | | | | LEU56 PRO139 |
| | | | | | ALA142 TRP143 |
| | | | | | PHE146 |

Our analysis also identified drugs that have been suggested, researched, or clinically administered in patients with KD42 and COVID-19 including dexamethasone, anakinra, aspirin, prednisolone, adalimumab, infliximab, and cyclosporine, indicating that our single-cell meta-analysis is successful in identifying key targets and therapeutic drugs that are effective (known) and new ones that may be tested for clinical translation.

All examples provided herein are intended for pedagogical purposes of aiding the reader in understanding the invention and the concepts contributed by the inventors to further the art, and are not to be construed as limitations to such specifically recited examples and conditions, nor does the organization of such examples in the specification relate to a showing of the superiority or inferiority of the invention. Although one or more embodiments of the present invention have been described in detail, it should be understood that the various changes, substitutions, and alterations could be made hereto without departing from the spirit and scope of the invention.

It is intended that the specification and examples be considered as examples only, with a true scope and spirit of the invention being indicated by the following claims.

What is claimed is:

1. A method of identifying specific drug candidates for disease treatment, the method comprising:
(A) Data Pre-processing: data acquisition of several single-cell RNA-seq data, newly generated or extracted from public genomic data repositories, followed by a series of data pre-processing steps, which include quality control, integration/harmonization, clustering, cell annotation, and disease-associated cell subtype identification;
(B) Integration and Clustering: All the pre-processed datasets are then merged, and the standard data processing workflow was repeated;
(C) Cell Annotation: Using the integrated data with defined clusters, specific cell identities are classified using automated cell annotation or reference mapping;
(D) Identification of Pathogenic cell type, molecular mechanisms driven by the pathogenic cell subtype, the critical modulators of disease mechanisms;
(E) Conducted cheminformatics approach to a nominate drug based on the DGIdb (Drug-Gene Interaction Database) which calculates query score and interaction score, further comparing the score and select the nominate drug to identifying potential drugs that may interact with the target(s); and
(F) Calculated the potential binding affinities via molecular docking (structure-based blind docking), further comparing the potential binding affinities to confirm and rank the interaction of the identified compounds against the targets for predicting one or more drug candidates.

2. The method of claim 1, wherein the Data Pre-processing further comprise standard scRNA-seq data pre-processing workflows, NormalizeData( ), FindVariableFeatures( ), ScaleData( ), RunPCA( ), and RunUMAP( ).

3. The method of claim 1, wherein the merged data of Integration and Clustering is integrated using Harmony package while performing necessary batch corrections, and clustering analysis is performed via FindNeighbors( ) and FindClusters( ) algorithms.

4. The method of claim 1, wherein the nominate drug which is selected according to a set of criteria, which comprises of (i) drugs targeting the disease-associated regulatory program, (ii) drugs targeting the key hub genes in disease-associated cell subtype, (iii) drugs targeting the important genes implicated by disease-gene association network analysis, (iv) drugs targeting the genes correlated with the disease-associated pathways, (v) drugs that are non-agonist/activating/potentiating and with DGIdb interaction score >0.2.

5. A computer program product tangibly embodying a set of instructions that, when executed by a processor, causes the processor to identify specific drug candidates for disease treatment by:
(A) Data Pre-processing: data acquisition of several single-cell RNA-seq data, newly generated or extracted from public genomic data repositories, followed by a series of data pre-processing steps, which include quality control, integration/harmonization, clustering, cell annotation, and disease-associated cell subtype identification;
(B) Integration and Clustering: All the pre-processed datasets are then merged, and the standard data processing workflow was repeated;
(C) Cell Annotation: Using the integrated data with defined clusters, specific cell identities are classified using automated cell annotation or reference mapping;
(D) Identification of Pathogenic cell type, molecular mechanisms driven by the pathogenic cell subtype, the critical modulators of disease mechanisms;
(E) Conducted cheminformatics approach to a nominate drug based on the DGIdb (Drug-Gene Interaction Database) which calculates query score and interaction score, further comparing the score and select the nominate drug to identifying potential drugs that may interact with the target(s); and
(F) Calculated the potential binding affinities via molecular docking (structure-based blind docking), further comparing the potential binding affinities to confirm and rank the interaction of the identified compounds against the targets for predicting one or more drug candidates.

6. The method of claim 5, wherein the Data Pre-processing further comprise standard scRNA-seq data pre-processing workflows, NormalizeData( ), FindVariableFeatures( ), ScaleData( ), RunPCA( ), and RunUMAP( ).

7. The method of claim 5, wherein the merged data of Integration and Clustering is integrated using Harmony package while performing necessary batch corrections, and clustering analysis is performed via FindNeighbors( ) and FindClusters( ) algorithms.

8. The method of claim 5, wherein the nominate drug which is selected according to a set of criteria, which comprises of (i) drugs targeting the disease-associated regulatory program, (ii) drugs targeting the key hub genes in disease-associated cell subtype, (iii) drugs targeting the important genes implicated by disease-gene association network analysis, (iv) drugs targeting the genes correlated with the disease-associated pathways, (v) drugs that are non-agonist/activating/potentiating and with DGIdb interaction score >0.2.

9. A method for treating neutrophilic inflammation-mediated disorder, the method comprises administering to the subject a drug, wherein the drug can target S10012 or TSPO proteins, wherein the drug is selected from a Methotrexate or a Metronidazole.

10. The method of claim 3, wherein the drug can target to an immune cell, which is CD177⁺ neutrophils.

11. The method of claim 3, wherein the methotrexate targets the S100A12 protein; the metronidazole targets the TSPO protein.

12. The method of claim 3, wherein the patient has increased levels of neutrophils higher than normal neutrophil counts.

13. The method of claim 3, wherein the treatment strategy suppresses CD177⁺ neutrophil-targeted pathways, including: CD177⁺ neutrophil expansion, neutrophil hyperactivation, neutrophil degranulation, neutrophil transendothelial transmigration, neutrophil extracellular trap (NET) formation, and reactive oxygen species/reactive nitrogen species (ROS/RNS) production, to substantially control the hyperactivated neutrophils in order to ameliorate, prevent, or inhibit systemic inflammation, organ damage, and cardiovascular disorders.

14. The method of claim 3, wherein the targets (S100A12 and TSPO) are critical hub genes common to mechanisms of claim 5.

15. The method of claim 3, wherein the patient has or is predisposed to symptoms of a neutrophilic inflammation-mediated disorder.

16. The method of claim 3, wherein the patients have acute, mild, moderate, or severe stages of a neutrophilic inflammation-mediated disorder.

17. The method of claim 3, wherein the drug is as a disease modulating strategy rather than a symptomatic treatment.

18. The method of claim 3, wherein the drug further can combine with one or more immunomodulatory or neutrophil-targeted drugs.

19. The method of claim 3, wherein the neutrophilic inflammation-mediated disorder is a neutrophil-related pathology or disease, wherein the neutrophil-related pathology or disease comprises Vasculitis, Kawasaki disease, Multisystem inflammatory syndrome in children, COVID-19, Neutrophilic Asthma, Chronic obstructive pulmonary disease (COPD), Systemic lupus erythematosus, Biliary atresia, Sepsis, Inflammatory bowel disease, Neutrophilic dermatoses, Chronic neutrophilic leukemia (CNL), Juvenile myelomonocytic leukemia (JMML), Cytokine storm, Acute respiratory distress syndrome (ARDS), Periodontitis, Bronchiectasis, or Cystic fibrosis (CF).
